(19) United States
(12) Patent Application Publication
(10) Pub. No.: US 2006/0241114 A1
(43) Pub. Date: Oct. 26, 2006

(54) Derivatives of azasugars as anticancer agents

(76) Inventors: Jasbir Singh Arora, Delhi (IN); Nidhi Gupta, Delhi (IN); Mohammad Salman, Plainsboro, NJ (US); Jang Bahadur Gupta, Haryana (IN); Upendra Kumar Pandit, Amsterdam (NL)

Correspondence Address: RANBAXY INC., 600 COLLEGE ROAD EAST, SUITE 2100, PRINCETON, NJ, 08540, US

(21) Appl. No.: 10/546,462

(22) PCT Filed: Feb. 20, 2003
(86) PCT No.: PCT/IB03/00619

§ 371 Date: Mar. 7, 2006

(51) Int. Cl. A61K 31/53; A61K 31/454; C07D 403/02

(52) U.S. Cl. 514/242; 514/253.11; 514/326; 514/327; 544/183; 544/360; 546/205; 546/210

(57) ABSTRACT

Certain derivatives of azasugars, useful in the treatment of cancer, are presented. This invention also relates to pharmacological compositions containing the compounds of present invention and treatment of cancer, including tumor or other neoplasm, with an azasugar.

### TECHNICAL FIELD

This invention relates to certain derivatives of azasugars, useful in the treatment of cancer. This invention also relates to pharmacological compositions containing the compounds of present invention and treatment of cancer, including tumor or other neoplasm, with an azasugar.

### BACKGROUND

During the past decade, azasugars have attracted attention of several organic and medicinal chemists because of their potential values as therapeutic agents for treatment of cancer, diabetes and AIDS. Main trends in known anticancer agents are drugs to kill malignant cells via cytotoxicity possessed by substances or via human immune system.

Antitumor therapy now involves an attack on the development of malignant tumor tissue by disrupting normal metabolic processes on which the new tumor depends for growth.

Many of the existing drugs, however are poorly tolerated by individuals such that the ratio of minimum dose with therapeutic effect to maximum dose that can be safely given is low. Moreover, it can be difficult to achieve a therapeutic concentration of these drugs in some regions of the body (e.g. brain cancer).

Doxorubicin, an anthracycline antibiotic is active against human neoplasms, including a variety of solid tumors, but is toxic and shows several adverse effects. Thus there is a need for more effective drugs to treat tumors and other neoplasia, especially to inhibit the growth thereof.

Compounds having anticancer activity have been described in U.S. Pat. No. 5,498,604 to Hasegawa et al., U.S. Pat. No. 4,985,445 to Tsuruoka et al., U.S. Pat. No. 5,250,545 to Tsuruoka et al., U.S. Pat. No. 6,225,325 to Jacob, WO 99/24401, WO 99/43675A1 and WO 00/56334. Structurally related compounds having antiviral and antibacterial activities have been discussed in U.S. Pat. No. 5,216,168 to Khanna et al. and WO 95/14028, respectively. Totally synthetic analogues of Siastatin B having inhibitory activity for tumor metastasis have been described by Satoh et al. in J. Antibiot., 47 (1), 101-107 (1994) and by Nishimura et al. in J. Antibiot., 49(3), 321-325 (1996). Preparation of seven-membered ring azasugars as glucosidase inhibitors and anticancer agents have been illustrated in Indian J. Chem., 38B, (1999), 1311-1321. New building blocks for tackling the synthesis of polyhydroxylated piperidines having related structure have been discussed in J. Org. Chem., (2000), 65, 7208-10.

### SUMMARY

The present invention is directed to the development of substances which can markedly inhibit metastasis of cancer cells and can be used for effective and proper treatment of cancer.

In one aspect, azasugar derivatives that inhibit metastasis of cancer cells are provided. In another aspect, processes for synthesis of such compounds are provided. In yet another aspect, pharmaceutical compositions containing such compounds which are useful in the treatment of cancer are provided. These compositions comprise an effective amount of at least one of such compounds.

The present invention also includes within its scope prodrugs of azasugar derivatives. In general, such prodrugs will be functional derivatives of these compounds which are readily converted in vivo into the defined compounds. Conventional procedures for the selection and preparation of suitable prodrugs are known.

The invention also includes the enantiomers, diastereomers, N-oxides and pharmaceutically acceptable salts of these compounds having anticancer activity. The invention further includes pharmaceutical compositions comprising azasugar derivatives, or prodrugs, metabolites, enantiomers, diastereomers, N-oxides, or pharmaceutically acceptable salts thereof, in combination with a pharmaceutically acceptable carrier and optionally included excipients.

In yet another aspect, the invention is directed to methods for treatment of cancer by delivering or administering to a mammal, an effective amount of such compounds.

Herein are provided azasugar derivatives represented by Formula I as shown below: 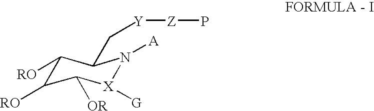
and its pharmaceutically acceptable salts, esters, enantiomers, diastereomers, N-oxides, prodrugs, metabolites, polymorphs or pharmaceutically acceptable solvates, wherein

A represents hydrogen, lower alkyl (C₁-C₄), lower alkenyl (C₁-C₄), or lower alkynyl (C₁-C₄);

X-G represents CO or CH₂;

R represents hydrogen, alkyl (C₁-C₄), acyl, aryl, aralkyl or trimethylsilyl;

Y represents O, NH or 5 to 6 membered cyclic ring optionally having one or more heteroatoms selected from the group N, O and S; 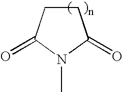
where n represents 0, 1 or 2; 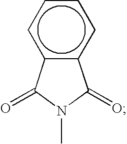 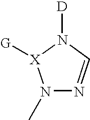
wherein X-G represents CO or CH₂, D represents an aryl group optionally substituted with F, Cl, Br and I;

Z represents CO, CS, SO₂, 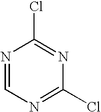
or no atom;

P represents no atom or straight or branched lower alkyl (C₁-C₄) which may be substituted with halogen selected from the group F, Cl, Br, I; trifluoromethyl; aryl which may be substitutted with one or more substituents selected from the group consisting of lower alkyl (C₁-C₃), halogen (F, Cl, Br, I); aralkyl, 5 to 6 membered heterocyclic ring with one or more hetero atoms selected from the group N, O and S; alkylamino in which the alkyl ring may be straight or branched;

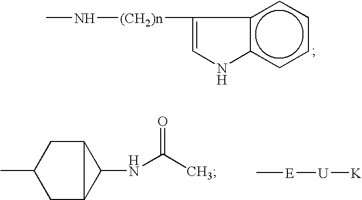

wherein E represents O or NH; U represents straight or branched lower alkyl (C₁-C₄) sulfonyl, adamantane, fused aryl rings or no atom; K represents 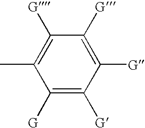
where G, G′, G″, G′″ and G′′ may be independently selected from hydrogen, lower alkyl (C₁-C₄), aryl which may optionally be substituted with one or more halogens selected from the group F, Cl, Br and I, CH₃, CF₃ OCH₃, COCH₃, NH₂ or NO₂; —CH₂-L where L represents 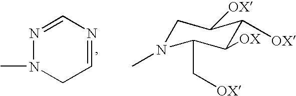
wherein X′ may be hydrogen, aryl or aralkyl;

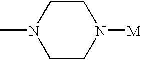

wherein M represents hydrogen, lower alkyl (C₁-C₄), pyrimidyl; aryl which may optionally be substituted with lower alkyl (C₁-C₃), trifluoromethyl or halogen which may be selected from the group F, Cl, Br and I; aralkyl;

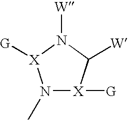

wherein X-G represents CO or CH₂, W′ W″ may independently be selected from hydrogen or may form a fused aryl ring of 6 carbon atoms; P or K can further be 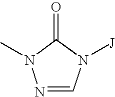
wherein J represents 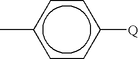
where Q represents halogen which may be selected from the group F, Cl, Br and I; or 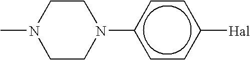
wherein Hal may be selected from the group F, Cl, Br and I; or P or K can further be

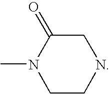

Unless otherwise defined, all technical and scientific terms used herein have the same ordinary meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The present invention also includes within its scope prodrugs of the disclosed compounds. In general, such prodrugs will be functionalized derivatives of these compounds, which are readily converted in vivo into the disclosed compounds. For example carboxylic acid esters can be formed from free hydroxyl groups on the azasugar compounds described herein, by reaction with carboxylic acids. Alternatively, carboxylic acid esters can be formed from free carboxylic acid groups (or their equivalents) on the azasugar compounds described herein, by reaction with alcohols. Also, amide linkages can be formed between either amino groups on the azasugar compounds and carboxylic acids, or between carboxylic acid groups on the azasugars and amines. The synthetic reactions to produce these linkages are well known to those of ordinary skill in the art. These ester and amide linkages can be hydrolysed, for example, by particular esterases and amidases known to those of ordinary skill in the art.

Other features and advantages of the invention will be apparent from the following detailed description, and from the claims.

### DETAILED DESCRIPTION OF THE INVENTION

The compounds of the present invention may be prepared by one of the reaction sequences (Schemes I-XI) to yield the compounds of Formula I. The starting materials for Schemes I-XI may be suitably adapted to produce the more specific compounds of Formula I.

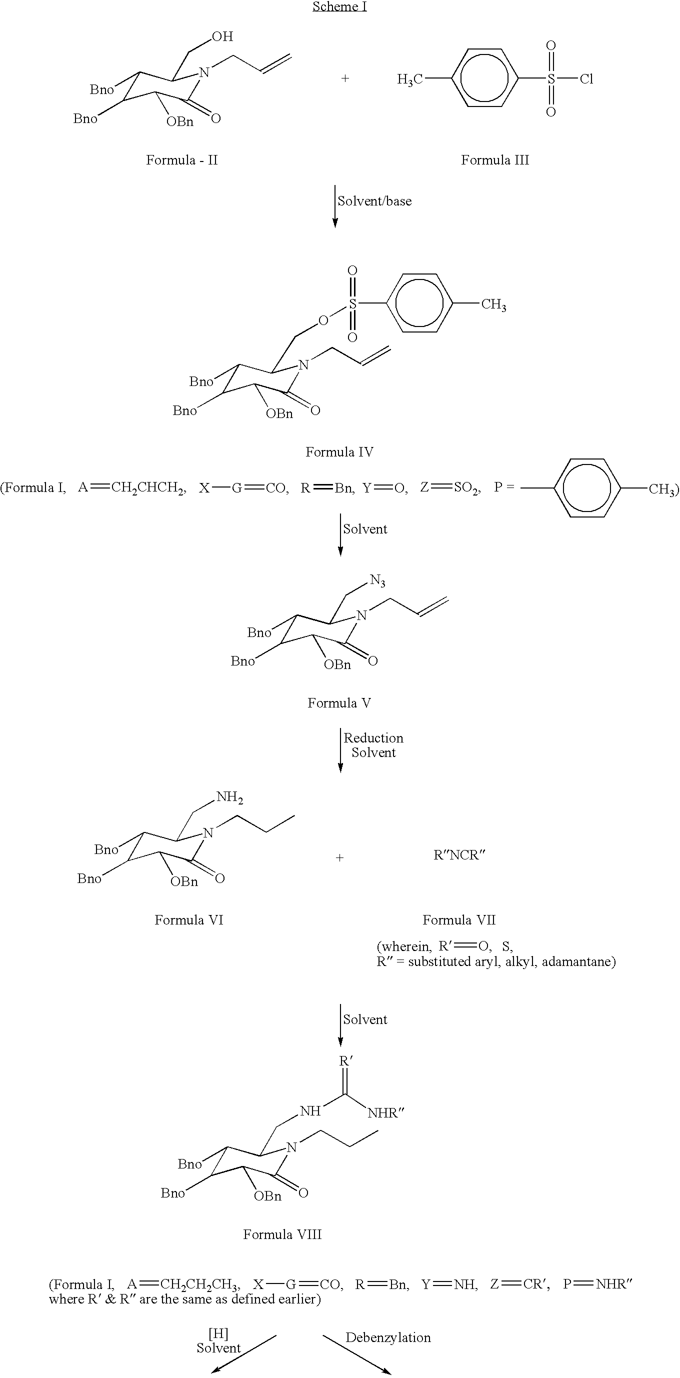

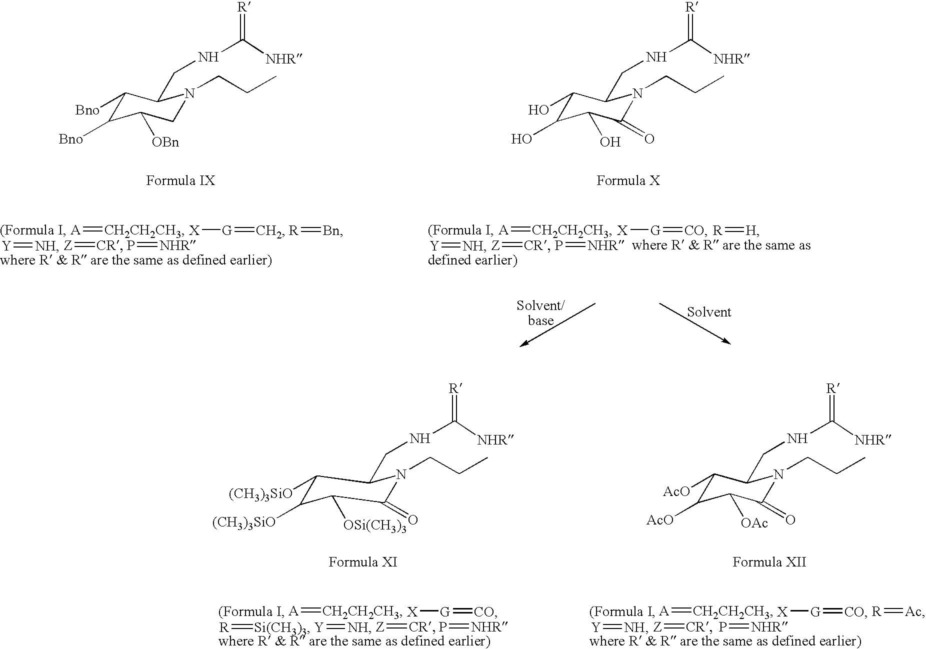

In Scheme I, the compound of Formula II can react with p-toluene sulphonyl chloride of Formula III in presence of a base and a solvent at a temperature ranging from about 0° to 60° C. for a period varying between 3-24 hours to produce the compound of Formula IV (Formula I, A is CH₂CHCH₂, X-G is CO, R is Bn (benzyl), Y is O, Z is SO₂,

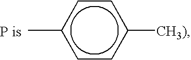

which on azidation in a suitable solvent at a temperature ranging from 20-80° C. for a period varying between 2-30 hours yields the compound of Formula V which further on catalytic reduction in a solvent yields the compound of Formula VI which on condensation with a compound of Formula VII in a suitable solvent at a temperature ranging from between about 10°-70° C. for a period varying between about 2-24 hours yields a compound of Formula VIII (Formula I, when A is CH₂CH₂CH₃, X-G is CO, R is Bn, Y is NH, Z is CR′, P is NHR″ where R and R″ are the same as defined earlier), this on reduction in a suitable solvent yields a compound of Formula IX (Formula I, when A is CH₂CH₂CH₃, X-G is CH₂, R is Bn, Y is NH, Z is CR′, P is NHR″ where R and R″ are the same as defined earlier), while on debenzylation in a suitable polar protic solvent for a time period ranging between about 2-40 hours yields a compound of Formula X (Formula I, when A is CH₂CH₂CH₃, X-G is CO, R is H, Y is NH, Z is CR′, P is NHR″ where R and R″ are the same as defined earlier), which on silylation in presence of a base and an organic solvent yields the compound of Formula XI (Formula I, when A is CH₂CH₂CH₃, X-G is CO, R is Si(CH₃)₃, Y is NH, Z is CR′, P is NHR″ where R and R″ are the same as defined earlier), while on acetylation in a suitable solvent at a temperature ranging from 0° C. to 60° C. for a period varying between 2 to several hours to yield a compound of Formula XII (Formula I, when A is CH₂CH₂CH₃, X-G is CO, R is Ac, Y is NH, Z is CR′, P is NHR″ where R and R″ are the same as defined earlier).

The tosylation of compound of Formula II can be carried out in presence of a base such as potassium carbonate, cesium carbonate, sodium carbonate, triethylamine or diisopropylamine. The solvent used in this reaction can be for example, dichloromethane, dichloroethane or chloroform. The temperature conditions can be, for example, 10°-20° C. The azidation of compound of Formula IV can be carried out in a solvent such as dimethyl sulfoxide, N,N-dimethylformamide, sulfolane, dimethyl acetamide, hexamethyl phosphoramide or N-methyl-2-pyrrolidone. The reduction of compound of Formula V can be carried out in a polar protic solvent such as methanol or ethanol. The condensation of the compound of Formula VI and a compound of Formula VII can be carried out in a suitable solvent such as tetrahydrofuran, acetone or acetonitrile at a temperature ranging from about 20° to 80° C. The reduction of the compound of Formula VIII can be carried out with lithium aluminium hydride, for example. The debenzylation of the compound of Formula VIII can be carried out in a polar protic solvent such as, for example, methanol or ethanol. The silylation of compound of Formula X can be carried out in an organic solvent such as dichloromethane, dichloroethane, tetrahydrofuran chloroform, acetone or acetonitrile. The base used in this reaction can be, for example, potassium carbonate, sodium carbonate, cesium carbonate, triethylamine and diisopropylamine. The acetylation of the compound of Formula X can be carried out in an organic solvent such as benzene, toluene, xylene or pyridine at a temperature ranging from about 20°-60° C.

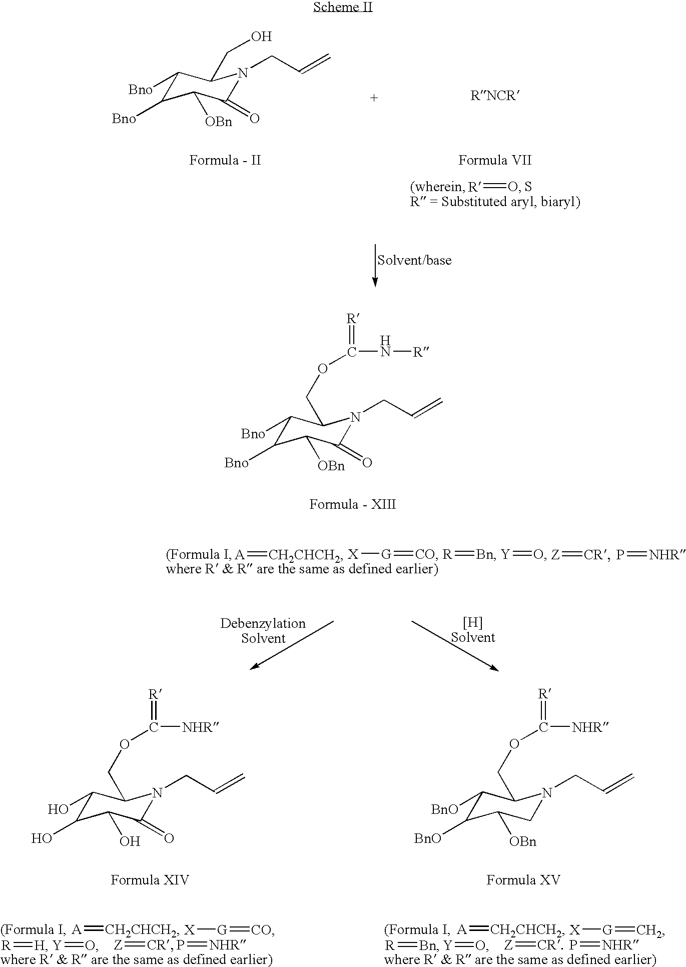

In Scheme II, the compound of Formula II reacts with a compound of Formula VII in presence of a base and a suitable solvent to give a compound of Formula XIII (Formula I, when A is CH₂CHCH₂, X-G is CO,R is H, Y is O, Z is CR′, P is NHR″ where R′ & R″ are the same as defined earlier) while the compound of Formula XIII on reduction in a suitable solvent for a period of one to several hours yields a compound of Formula XV (Formula I, when A is CH₂CHCH₂, X-G is CH₂, R is Bn, Y is O, Z is CR′, P is NHR″ where R′ & R″ are the same as defined earlier).

The reaction of compound of Formula II and the compound of Formula VII can be carried out in presence of a base such as triethylamine, diisopropylamine, potassium carbonate, cesium carbonate or sodium carbonate. The reaction can be carried out in a solvent such as acetone, acetonitrile or tetrahydrofuran. The debenzylation of Formula XIII can be carried out in a polar protic solvent such as methanol or ethanol. The reduction of compound of Formula XIII can be carried out in a solvent such as tetrahydrofuran, acetone or acetonitrile. Lithium aluminium hydride is a suitable reducing agent.

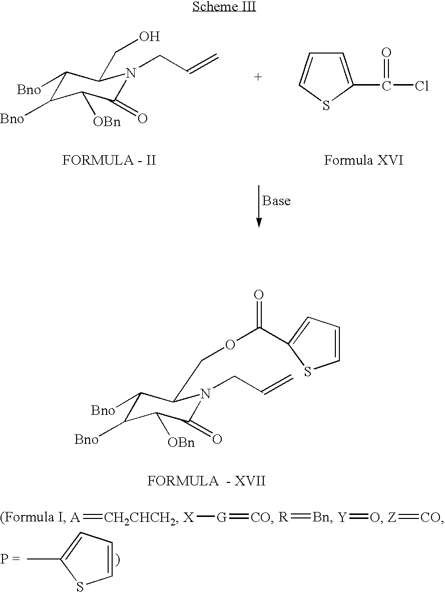

Scheme III, depicts a synthesis of compounds of Formula XVII (Formula I, A is CH₂CHCH₂, X-G is CO, R is Bn, Y is O, Z is CO, P is 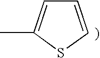
which includes condensing the compound of Formula II with the compound Formula XVI in the presence of a base at a temperature ranging from about 20-60° C. for a period varying between about 3-24 hours to give the compound of Formula XVII.

The base can be, for example, triethylamine or diisopropylamine. A suitable temperature range is 30-35° C.

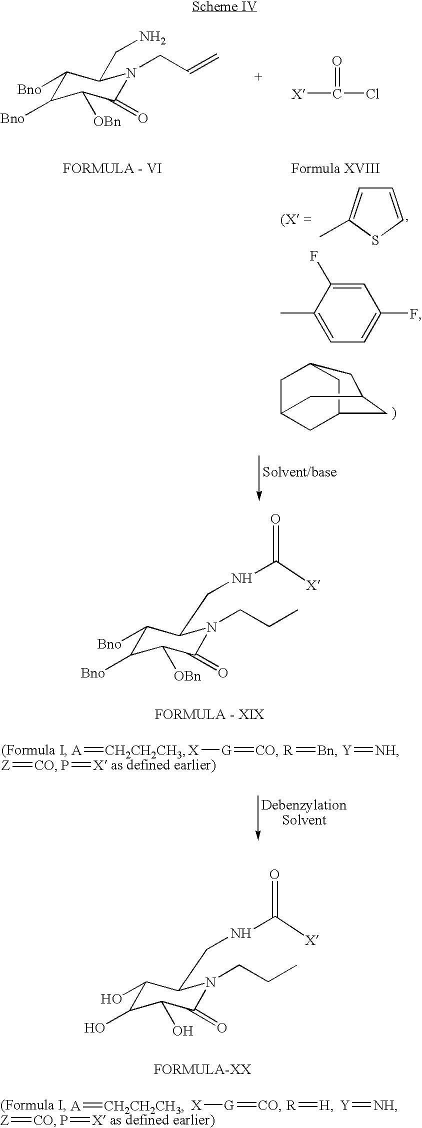

In Scheme IV the compound of Formula VI reacts with a compound of Formula XVIII in presence of a base and an organic solvent for a period of one to several hours to yield a compound of Formula XIX (Formula I, when A is CH₂CH₂CH₃, X-G is CO, R is Bn, Y is NH, Z is CO, P is X′, same as defined earlier) which on debenzylation in a polar protic solvent for a period of 2-40 hours yields a compound of Formula XX (Formula I, A is CH₂CH₂CH₃, X-G is CO, R is H, Y is NH, Z is CO, P is X′ same as defined earlier)

The reaction of compound of Formula VI and a compound of Formula XVIII can be carried out in presence of a base such as triethylamine or diisopropylamine. The organic solvent used in the reaction can be, for example, dichloromethane, dichloroethane, chloroform, tetrahydrofuran or acetone. The debenzylation is carried out in a polar protic solvent such as methanol or ethanol.

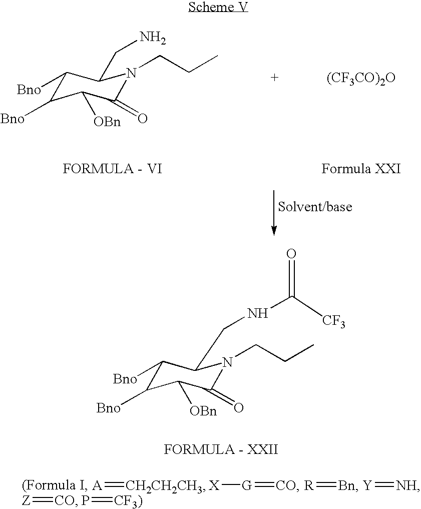

Scheme V reveals the synthesis of compound of Formula XXII (Formula I, when A is CH₂CH₂CH₃, X-G is CO, R is Bn, Y is NH, Z is CO, P is CF₃) which includes condensing the compound of Formula VI with the compound of Formula XXI in presence of a base and an organic solvent at a temperature ranging from 0° to 70° C. for a period varying between one to several hours to give the compound of Formula XXII.

The suitable organic solvent can be, for example, dichloromethane, dichloroethane or chloroform. The reaction is carried out in the presence of a base such as triethylamine or diisopropylamine. A suitable temperature range is 15-25° C.

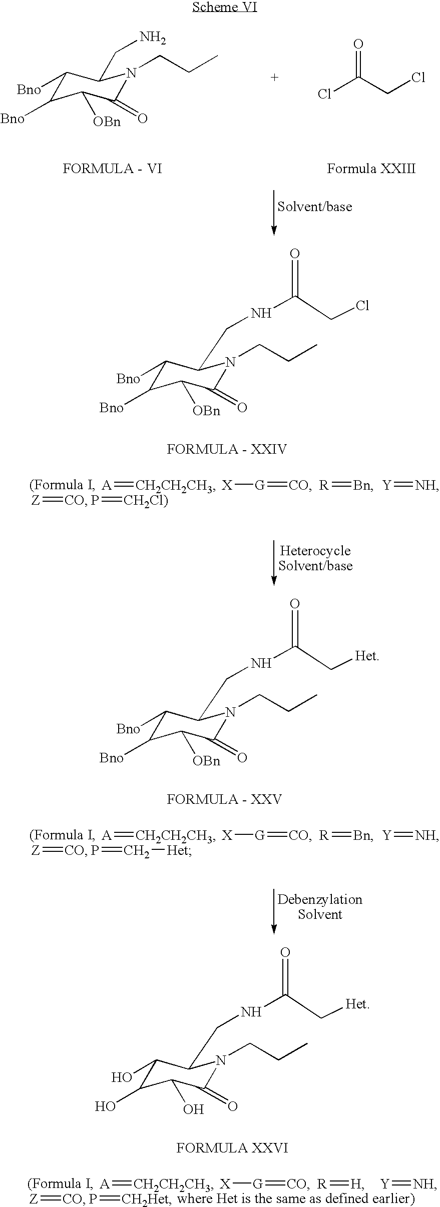

wherein Het can be:

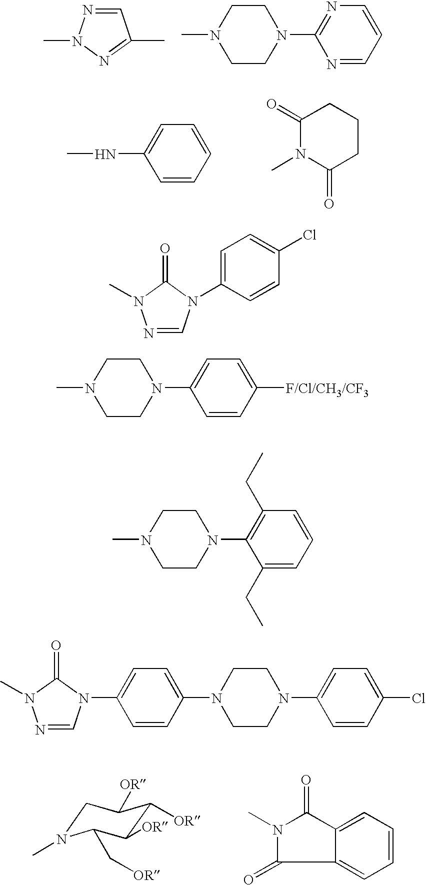

In Scheme VI, the compound of Formula VI reacts with the compound of Formula XXIII in presence of a base and a suitable solvent at a temperature ranging from 0° to 60° for a period varying from one to several hours to give the compound of Formula XXIV (Formula L when A is CH₂CH₂CH₃, X-G is CO, R is Bn, Y is NH, Z is CO, P is CH₂Cl) which on reaction with different heterocycles in presence of a base and phase transfer catalyst in a suitable solvent for a period ranging from 5 to 24 hours yields a compound of Formula XXV (Formula I, A is CH₂CH₂CH₃, X-G is CO, R is Bn, Y is NH, Z is CO, P is CH₂Het where Het is the same as defined in Scheme VI) which is further subjected to debenzylation in a protic polar solvent for a period of 2-40 hours to give the corresponding compounds of Formula XVI (Formula I, A is CH₂CH₂CH₃, X-G is CO, R is H, Y is NH, Z is CO, P is CH₂Het where Het is defined above).

The solvent used for the reaction of compound of Formula VI and compound of Formula XIII can be, for example, dichloromethane, dichloroethane, chloroform, tetrahydrofuran or acetone. The base can be, for example, triethylamine or diisopropylamine. Suitable temperature conditions are 20-30° C. The condensation of compound of Formula XXIV with heterocycle is carried out in an aprotic solvent such as tetrahydrofuran or acetone in presence of a phase transfer catalyst, for example tributylammonium iodide. The base used in the reaction can be potassium carbonate, sodium carbonate and cesium carbonate. The debenzylation of compound of Formula XXV is carried out in a polar protic solvent such as methanol or ethanol.

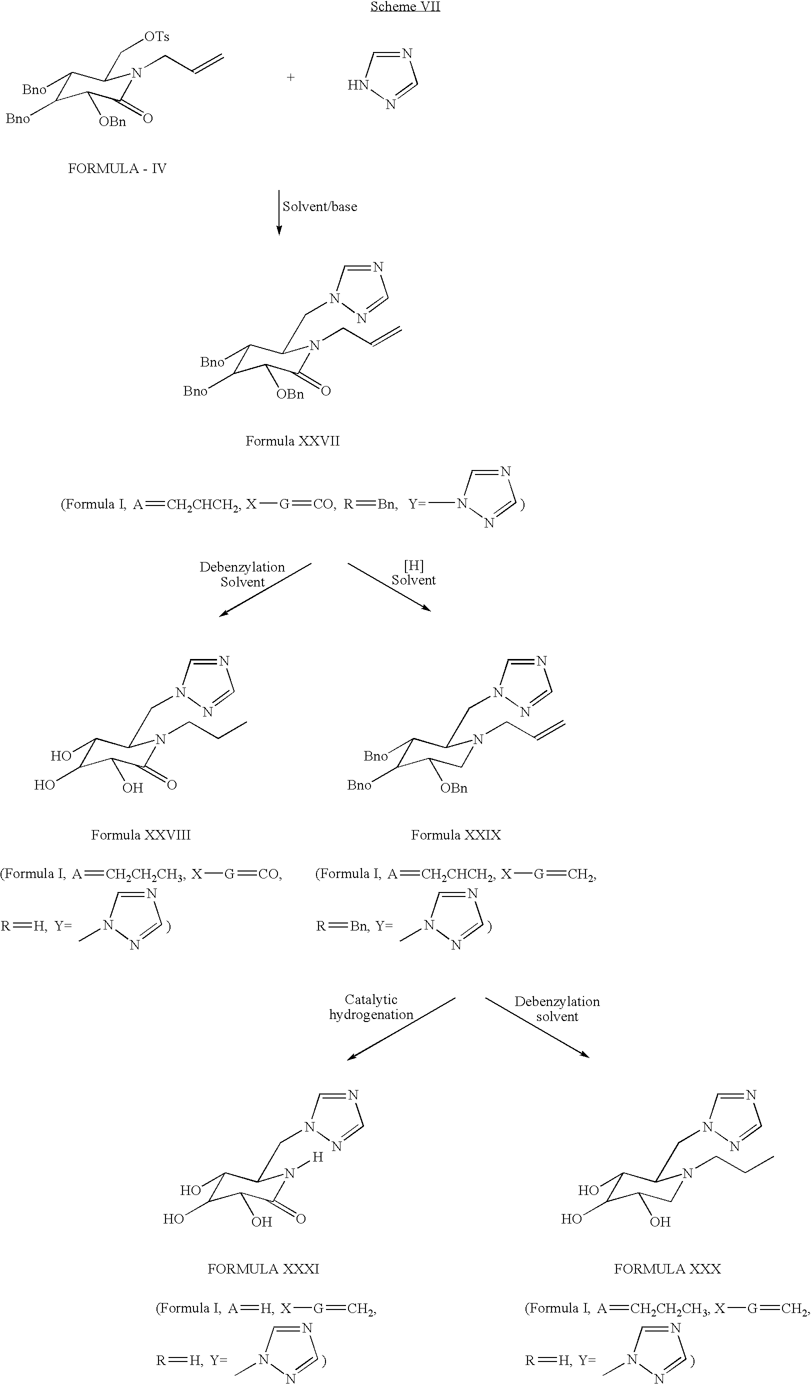

In Scheme VII, the compound of Formula IV reacts with triazole in presence of a base and a dipolar aprotic solvent at a temperature ranging from 20-80° C. for a period of 2-24 hours to give the compound of Formula XXVII, (Formula I, A is CH₂CHCH₂, X-G is CO, R is Bn, 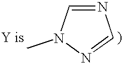
which on debenzylation in a polar protic solvent for a period of 2-40 hours yields a compound of Formula XXVII (Formula I, A is CH₂CH₂CH₃, X is CO, R is H, 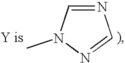
while on reduction in a suitable solvent, the compound of Formula XXVII is converted to a compound of Formula XXIX (Formula I, A is CH₂CHCH₂, X-G is CH₂, R is Bn, 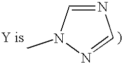
which is on debenzylation in a suitable solvent yields the compound of Formula XXX (Formula I, A is CH₂CH₂CH₃, X-G is CH₂, R is H, 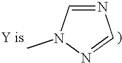
while when subjected to catalytic hydrogenation in a polar protic solvent for a period of one to several hours to yields the compound of Formula XXXI (Formula I, A is H, X-G is CH₂,R is H,

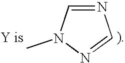

A suitable solvent is a dipolar aprotic protic solvent for the reaction of compound of Formula IV and triazole. For example, dimethyl sulfoxide, N,N-dimethyl formamide or dimethyl acetamide can be employed. The inorganic base used in the reaction can be potassium carbonate, sodium carbonate or cesium carbonate. The reduction of compound of Formula XXVII is carried out in a solvent such as tretrahydrofuran, diethylether or acetone. A suitable reducing agent is lithium aluminum hydride. The dehydrogenation of compounds of Formula XXVII and Formula XXIX can be carried out in a polar protic solvent such as methanol or ethanol. The catalytic hydrogenation of compound of Formula XXIX can be carried out with Pd/C (Palladium/carbon) in a polar protic solvent, such as methanol or ethanol.

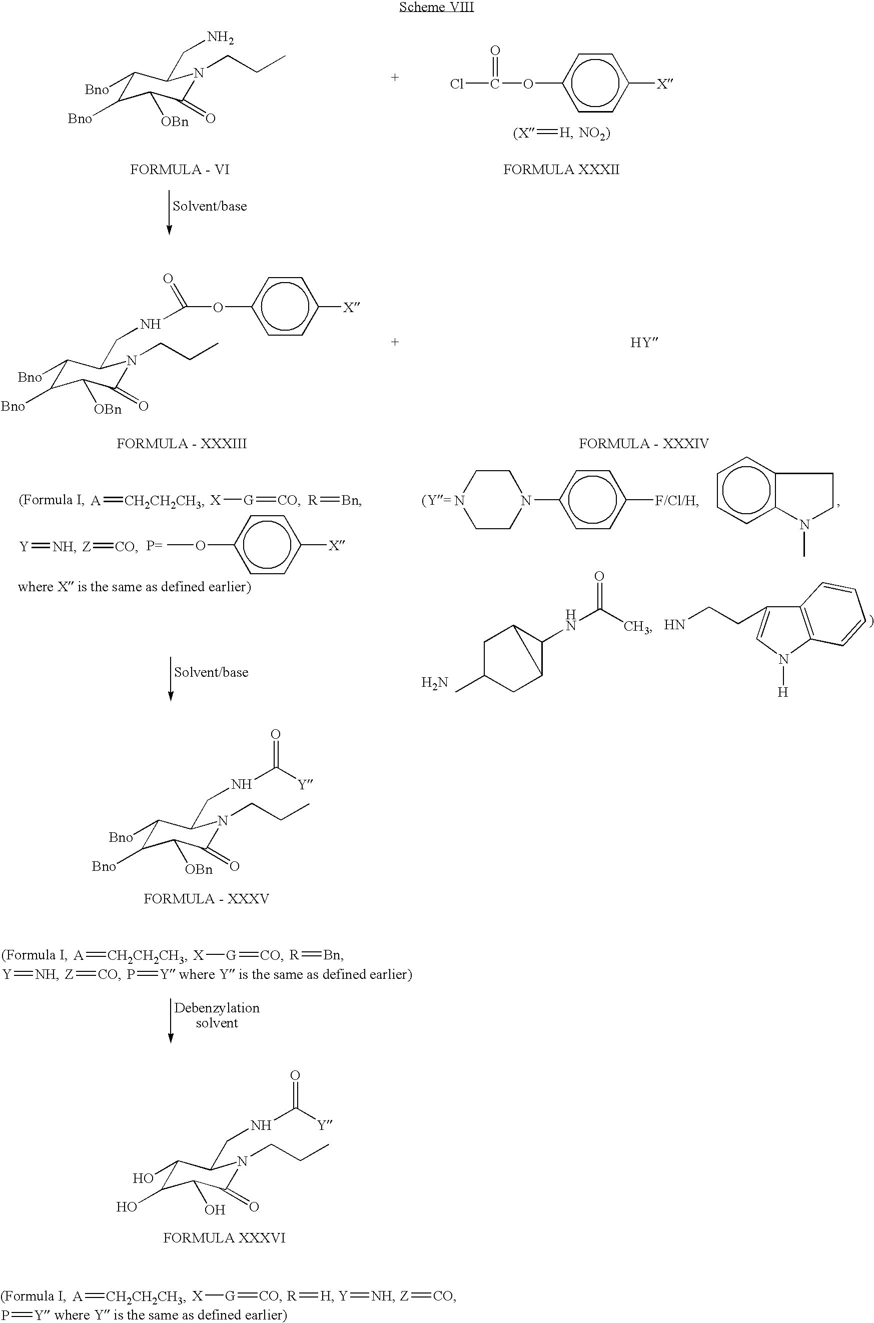

In Scheme VIII, the compound of Formula VI reacts with the compound of Formula XXXII in presence of a base and a suitable solvent at a temperature ranging from −60° C. to 60° C. for a period varying from one to several hours to give the compound of Formula XXXIII (Formula I, A is CH₂CHCH₃, X-G is CO, R is Bn, Y is NH, Z is CO, P is 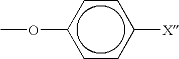
where X is the same as defined earlier) which further condenses with a compound of Formula XXXIV in presence of a base and an organic solvent for a period ranging from one to several hours to give a compound of Formula XXXV (Formula I, when A is CH₂CHCH₃, X is O, R is Bn, Y is NH, Z is CO, P is Y″ where Y″ is the same as defined earlier) which is subjected to debenzylation in polar solvent for a period varying between 2-40 hours to yield a compound of Formula XXXVI (Formula I, when A is A is CH₂CH₂CH₃, X-G is CO, R is H, Y is NH, Z is CO, P is Y″ where Y″ is the same as defined earlier).

The base used in the reaction of compounds of Formula VI and compound of Formula XXXII can be, for example, triethylamine or diisopropylamine. The solvent can be, for example, dichloromethane, dichloroethane or chloroform. The reaction of compound of Formula XXXIII and compound of Formula XXXIV is carried out in an organic solvent such as dichloromethane, dichloroethane or chloroform. The base in the reaction can be, for example, triethylamine or diisopropylamine. The debenzylation of compound of Formula XXXV is carried out in a polar protic solvent such as methanol or ethanol.

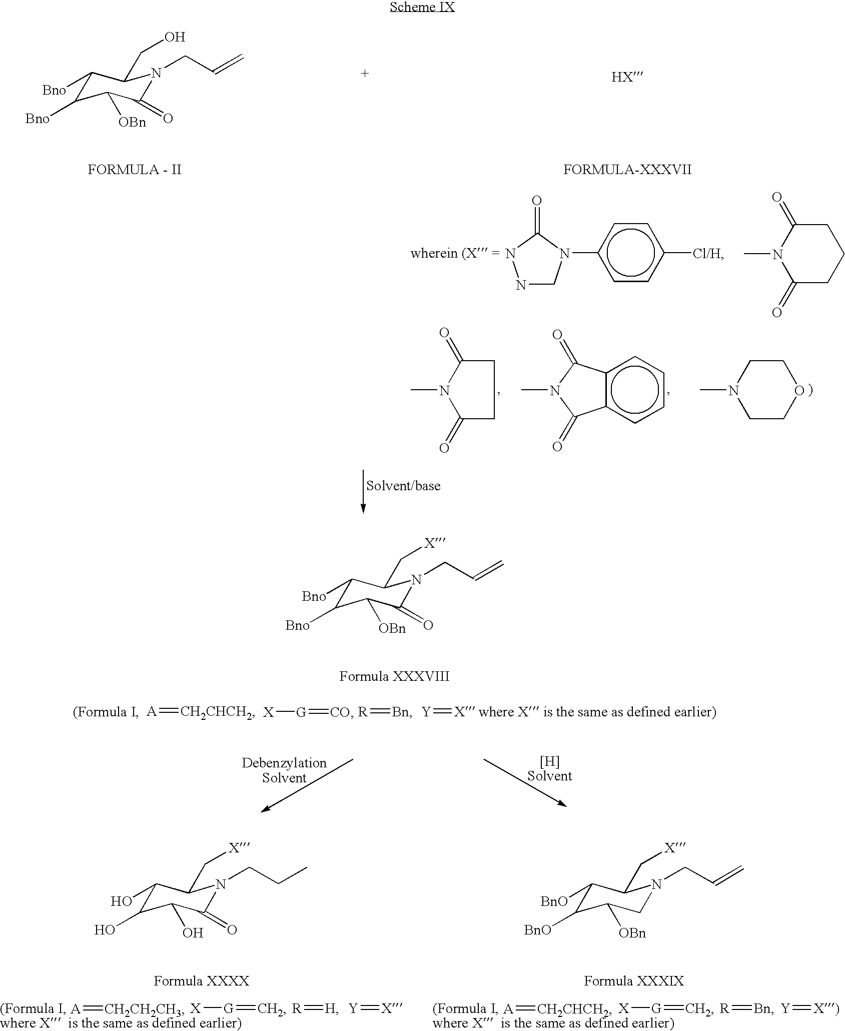

Scheme IX, shows the synthesis of compound of Formula XXVIII (Formula I, A is CH₂CHCH₂, X-G is CO, R is Bn, Y is X′″ as described earlier) which comprises reacting the compound of Formula II with a compound of Formula XXXVII in a suitable solvent to give the compound of Formula XXXVIII (Formula I, when A is CH₂CHCH₂, X is G is CO, R is Bn, Y is X′″ as defined earlier) which on reduction in a appropriate solvent gives a compound of Formula XXXIX (Formula I, A is CH₂CHCH₂, X-H is CH₂, R is Bn, Y is X′″ as defined earlier) while, on debenzylation in a polar solvent it gives a compound of Formula XXXX (Formula I, A is CH₂CH₂CH₃, X-G is CH₂, R is H, Y is X′″ as defined earlier).

The reaction of compound of Formula II and compound of Formula XXXVII is carried out in a solvent such as tetrahydrofuran, acetone or acetonitrile. The base used in the reaction can be, for example, triphenylphosphine or triethylamine. A suitable temperature range is 20-25° C. The reduction of compound of Formula VIII is carried out in a solvent such as tetrahydrofuran, diethylether or acetone. The reducing agent can be, for example, lithium aluminum hydride. The debenzylation of the comound of Formula XXXVIII can be carried out in a polar protic solvent, such as methanol or ethanol.

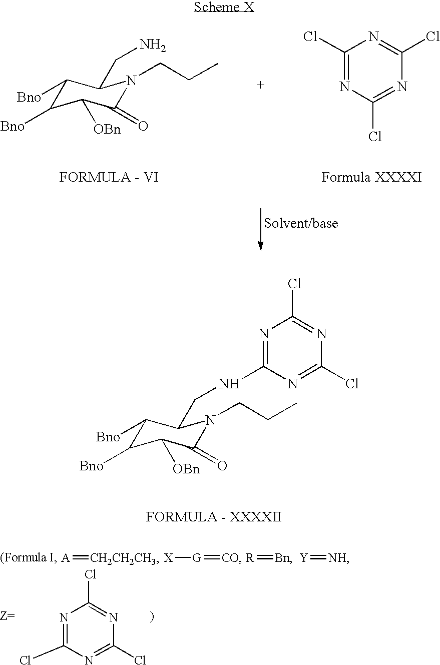

Scheme X reveals the synthesis of compound of Formula XXXXII (Formula I, when A is CH₂CH₂CH₂, X-G is CO, R is Bn, Y is NH, 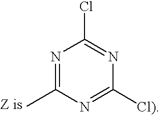
The preparation comprises condensing the compound of Formula II with cyanuric chloride of Formula XXXXI in a suitable solvent and a suitable base to give the compound of Formula XXXXII.

Suitable solvents can be, for example, acetone, tetrahydrofuran or acetonitrile. The reaction is carried out in the presence of a base, such as potassium carbonate, sodium carbonate or cesium carbonate. Suitable temperature conditions for the reaction are 20-30° C.

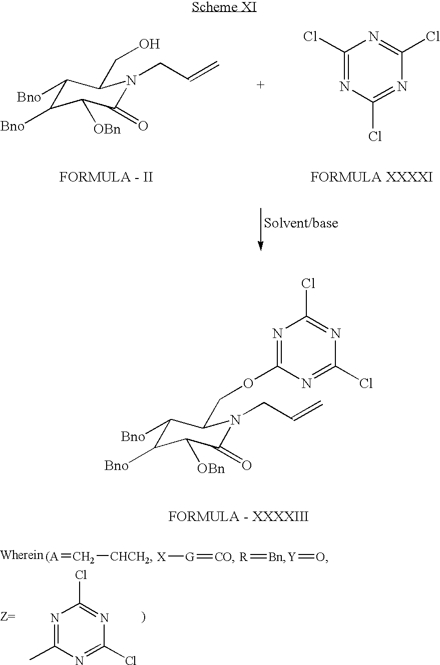

The compound of Formula XXXXIII (Formula I, when A is CH₂CHCH₂, X-G is CO, R is Bn, Y is O, 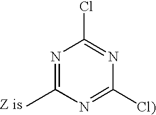
can be prepared by condensation of compound of Formula II with cyanuric chloride of Formula XXXXI in a suitable solvent and a suitable base as depicted in Scheme XI.

The reaction can be carried out in a suitable solvent such as acetone or acetonitrile. The reaction can be carried out in presence of a base such as potassium carbonate, sodium carbonate or cesium carbonate. A suitable temperature range is 10-20° C.

In the above schemes, where specific bases, solvents, phase transfer catalysts, etc., are mentioned, it is to be understood that other bases, solvents, phase transfer catalysts, etc., known to those skilled in the art may also be used. Similarly, the reaction temperature and duration of the reactions may be adjusted according to the desired needs.

Particular molecules which exemplify compounds of Formula I are given below:
N-Allyl-6-O-(p-toluenesulphonyl)-2,3,4-tri-O-benzyl-D-gluco-δ-lactam (Compound No. 1)
2,3,4-Tri-O-benzyl-6-N-{[2-naphthyl]aminothiocarbonylamino}-N-propyl-D-gluco-δ-lactam (Compound No. 2)
2,3,4-Tri-O-benzyl-6-N-{[4-chlorophenyl]aminocarbonylamino}-N-propyl-D-gluco-δ-lactam (Compound No. 3)
2,3,4-Tri-O-benzyl-6-N-{[p-methoxyphenyl]aminocarbonylamino}-N-propyl-D-gluco-δ-lactam (Compound No. 4)
2,3,4-Tri-O-benzyl-6-N-{[p-nitrophenyl]aminocarbonylamino}-N-propyl-D-gluco-δ-lactam (Compound No. 5)
2,3,4-Tri-O-benzyl-6-N-{[p-tolyl]aminocarbonylamino}-N-propyl-D-gluco-δ-lactam (Compound No. 6)
2,3,4-Tri-O-benzyl-6-N-{[4-chloro-2-trifluoromethylphenyl]aminocarbonylamino}-N-propyl-D-gluco-δ-lactam (Compound No. 7)
2,3,4-Tri-O-benzyl-6-N-{[isopropyl]aminothiocarbonylamino}-N-propyl-D-gluco-δ-lactam (Compound No. 8)
2,3,4-Tri-O-benzyl-6-N-{[phenyl]aminothiocarbonylamino}-N-propyl-D-gluco-δ-lactam (Compound No. 9)
2,3,4-Tri-O-benzyl-6-N-{[phenyl]aminocarbonylamino}-N-propyl-D-gluco-δ-lactam (Compound No. 10)
2,3,4-Tri-O-benzyl-6-N-{[p-fluorophenyl]aminocarbonylamino}-N-propyl-D-gluco-δ-lactam (Compound No. 11)
2,3,4-Tri-O-benzyl-6-N-{[p-chlorophenylsulfonyl]aminocarbonylamino}-N-propyl-D-gluco-δ-lactam (Compound No. 12)
2,3,4-Tri-O-benzyl-6-N-{[3-chlorophenyl]aminocarbonylamino}-N-propyl-D-gluco-δ-lactam (Compound No. 13)
2,3,4-Tri-O-benzyl-6-N-{[3-acetylphenyl]aminocarbonylamino}-N-propyl-D-gluco-δ-lactam (Compound No. 14)
2,3,4-Tri-O-benzyl-6-N-{[3-chloro-6-methoxyphenyl]aminocarbonylamino}-N-propyl-D-gluco-δ-lactam (Compound No. 15)
2,3,4-Tri-O-benzyl-6-N-{[2,4-difluorophenyl]aminocarbonylamino}-N-propyl-D-gluco-δ-lactam (Compound No. 16)
2,3,4-Tri-O-benzyl-6-N-{[2,4,6-trichlorophenyl]aminocarbonylamino}-N-propyl-D-gluco-δ-lactam (Compound No. 17)
2,3,4-Tri-O-benzyl-6-N-{[3,4-dichlorophenyl]aminocarbonylamino}-N-propyl-D-gluco-δ-lactam (Compound No. 18)
2,3,4-Tri-O-benzyl-6-N-{[2,4-dichlorophenyl]aminocarbonylamino}-N-propyl-D-gluco-δ-lactam (Compound No. 19)
2,3,4-Tri-O-benzyl-6-N-{[isopropyl]aminocarbonylamino}-N-propyl-D-gluco-δ-lactam (Compound No. 20)
2,3,4-Tri-O-benzyl-6-N-{[4-chlorophenyl]aminothiocarbonylamino}-N-propyl-D-gluco-δ-lactam (Compound No. 21)
2,3,4-Tri-O-benzyl-6-N-{[4-trifluoromethylphenyl]aminocarbonylamino}-N-propyl-D-gluco-δ-lactam (Compound No. 22)
2,3,4-Tri-O-benzyl-6-N-{[1-adamantyl]aminocarbonylamino}-N-propyl-D-gluco-δ-lactam (Compound No. 23)
N-Propyl-6-N-{[phenyl]aminocarbonylamino}-D-gluco-δ-lactam (Compound No. 24)
6-N-{[2-Trifluoromethylphenyl]aminocarbonylamino}-N-propyl-D-gluco-δ-lactam (Compound No. 25)
6-N-{[p-Tolyl]aminocarbonylamino}-N-propyl-D-gluco-δ-lactam (Compound No. 26)
6-N-{[p-Methoxyphenyl]aminocarbonylamino}-N-propyl-D-gluco-δ-lactam (Compound No. 27)
6-N-{[p-Aminophenyl]aminocarbonylamino}-N-propyl-D-gluco-δ-lactam (Compound No. 28)
6-N-{[4-Fluorophenyl]aminocarbonylamino}-N-propyl-D-gluco-δ-lactam (Compound No. 29)
6-N-{[Isopropyl]aminocarbonylamino}-N-propyl-D-gluco-δ-lactam (Compound No. 30)
6-N-{[4-Trifluoromethylphenyl]aminocarbonylamino}-N-propyl-D-gluco-δ-lactam (Compound No. 31)
2,3,4-Tri-O-acetyl-6-N-{[4-methoxyphenyl]aminocarbonylamino}-N-propyl-D-gluco-δ-lactam (Compound No. 32)
2,3,4-Tri-O-acetyl-6-N-{[phenyl]aminocarbonylamino}-N-propyl-D-gluco-δ-lactam (Compound No. 33)
2,3,4-Tri-O-acetyl-6-N-{[4-fluorophenyl]aminocarbonylamino}-N-propyl-D-gluco-δ-lactam (Compound No. 34)
2,3,4-Tri-O-trimethylsilyl-6-N-{[phenyl]aminocarbonylamino}-N-propyl-D-gluco-δ-lactam (Compound No. 35)
2,3,4-Tri-O-benzyl-6-N-{[4-chlorophenyl]aminocarbonylamino}-1,5-dideoxy-1,5-imino glucitol (Compound No. 36)
2,3,4-Tri-O-benzyl-6-N-{[2,4-difluorophenyl]aminocarbonylamino}-1,5-dideoxy-1,5-imino glucitol (Compound No. 37)
2,3,4-Tri-O-benzyl-6-N-{[isopropyl]aminocarbonylamino}-1,5-dideoxy-1,5-imino-N-propyl glucitol (Compound No. 38)
2,3,4-Tri-O-benzyl-6-N-{[4-chlorophenyl]aminothiocarbonylamino}-1,5-dideoxy-1,5-imino-N-propyl glucitol (Compound No. 39)
2,3,4-Tri-O-benzyl-6-N-{[4-fluorophenyl]aminocarbonylamino}-1,5-dideoxy-1,5-imino-N-propyl glucitol (Compound No. 40)
N-Allyl-6-O-(4-chlorophenylcarbamate)-2,3,4-tri-O-benzyl-D-gluco-δ-lactam (Compound No. 41)
N-Allyl-2,3,4-Tri-O-benzyl-6-(2-naphthylithiocarbamate)-D-gluco-δ-lactam (Compound No. 42)
N-Propyl-6-(phenylcarbamate)-N-propyl-D-gluco-δ-lactam (Compound No. 43)
N-Allyl-2,3,4-tri-O-benzyl-6-{p-chlorophenylcarbamate}-1,5-dideoxy-1,5-imino glucitol (Compound No. 44)
N-allyl-6-O-(2-thiophenyl)-2,3,4-tri-O-benzyl-D-gluco-δ-lactam (Compound No. 45)
2,3,4-Tri-O-benzyl-N-propyl-6-{[2-thiophene]carboxamido}-D-gluco-δ-lactam (Compound No. 46)
2,3,4-Tri-O-benzyl-6-N-{[2,4-difluorophenyl]carboxamido}-N-propyl-D-gluco-δ-lactam (Compound No. 47)
2,3,4-Tri-O-benzyl-6-(adamantanecarboxamido}-N-propyl-D-gluco-δ-lactam (Compound No. 48)
6-N-{[2,4Difluorophenyl]aminocarbonylamino}-N-propyl-D-gluco-δ-lactam (Compound No. 49)
6-(Adamantane carboxamido)-N-propyl-D-gluco-δ-lactam (Compound No. 50)
2,3,4-Tri-O-benzyl-6-(trifluoromethylacetamido)-N-propyl-D-gluco-δ-lactam (Compound No. 51)
2,3,4-Tri-O-benzyl-6-N-{2-[chloro]-acetyl}-N-propyl-D-gluco-δ-lactam (Compound No. 52)
2,3,4-Tri-O-benzyl-N-propyl-6-{2-[triazolyl]-acetyl}-D-gluco-δ-lactam (Compound No. 53)
2,3,4-Tri-O-benzyl-6-N-{2-[4-(pyrimidyl)piperazinyl]acetyl}-N-propyl-D-gluco-δ-lactam (Compound No. 54)
2,3,4-Tri-O-benzyl-6-N-{2-[4-fluoroanilino]acetyl}-N-propyl-D-gluco-δ-lactam (Compound No. 55)
2,3,4-Tri-O-benzyl-6-{2-[2,6-diketopiperidino]acetyl}-N-propyl-D-gluco-δ-lactam (Compound No. 56)
2,3,4-Tri-O-benzyl-6-N-{2-[4-chlorophenyl-3-(2H,4H)-1,2,4-triazol-3-onyl]acetyl}-N-propyl-D-gluco-δ-lactam (Compound No. 57)
2,3,4-Tri-O-benzyl-6-N-{2-[4-(4-fluorophenyl)-piperazin-1-yl]acetyl}-N-propyl-D-gluco-δ-lactam (Compound No. 58)
2,3,4-Tri-O-benzyl-6-N-{2-[N-(methyl)piperazin-1-yl]acetyl}-N-propyl-D-gluco-δ-lactam (Compound No. 59)
2,3,4-Tri-O-benzyl-6-N-{2-[(4-(4-(chlorophenyl)-piperazin-1-yl)-phenyl)-3(2H,4H)-1,2,4-triazol-3-onyl]acetyl}-H-propyl-D-gluco-δ-lactam (Compound No. 60)
2,3,4-Tri-O-benzyl-6-N-{2-[2,3,4,6-tetra-O-benzyl-1,5-dideoxy-1,5-imino-glucitolyl]acetyl}-N-propyl-D-gluco-δ-lactam (Compound No. 61)
2,3,4-Tri-O-benzyl-6-N-{2-[N-(2,6-diethylphenyl)piperazinyl]acetyl}-N-propyl-D-gluco-δ-lactam (Compound No. 62)
2,3,4-Tri-O-benzyl-6-{2-[1H-isoindole-1,3(2H)-diketo]acetyl}-D-gluco-δ-lactam (Compound No. 63)
2,3,4-Tri-O-benzyl-6-N-{2-[4-(4-chlorophenyl)piperazinyl]acetyl}-N-propyl-D-gluco-δ-lactam (Compound No. 64)
2,3,4-Tri-O-benzyl-6-N-{2-[4-(3-(trifluoromethyl)phenyl)piperazin-1-yl]acetyl}-N-propyl-D-gluco-δ-lactam (Compound No. 65)
N-Propyl-6-{2-[triazolyl]-acetyl}-D-gluco-δ-lactam (Compound No. 66)
6-N-{2-[1,5-Dideoxy-1,5-imino-glucit-6-ol]acetyl}-N-propyl-D-gluco-δ-lactam (Compound No. 67)
6-N-{2-[(N-Methyl)piperazinyl]acetyl}-N-propyl-D-gluco-δ-lactam (Compound No. 68)
N-Allyl-2,3,4-tri-O-benzyl-6-triazolyl-D-gluco-δ-lactam (Compound No. 69)
6-Triazolyl-N-propyl-D-gluco-δ-lactam (Compound No. 70)
N-Allyl-2,3,4-tri-O-benzyl-1,5-dideoxy-1,5-imino-D-glucitol (Compound No. 71)
1,5-Dideoxy-1,5-imino-6-triazolyl-glucitol (Compound No. 72)
1,5-Dideoxy-1,5-imino-N-propyl-6-triazolyl glucitol (Compound No. 73)
2,3,4-Tri-O-benzyl-6-N-[phenyl carbamate]-N-propyl-D-gluco-δ-lactam (Compound No. 74)
2,3,4-Tri-O-benzyl-6-N-{4-[4-chlorophenyl]piperazinyl carboxamido}-N-propyl-D-gluco-δ-lactam (Compound No. 75)
2,3,4-Tri-O-benzyl-6-N-{4-[4-fluorophenyl]piperazinyl carboxamido}-N-propyl-D-gluco-δ-lactam (Compound No. 76)
2,3,4-Tri-O-benzyl-6-N-{1,2-dihydro(2H)-indolyl carboxamido}-N-propyl-D-gluco-δ-lactam (Compound No. 77)
2,3,4-Tri-O-benzyl-6-N-[3-{2-iminocarbonyl aminoethyl)-indolyl]-N-propyl-D-gluco-δ-lactam (Compound No. 78)
2,3,4-Tri-O-benzyl-6-N-{(1α,5α,6α)-6-acetylamino-azabicyclo[3.1.0]hexyl carboxamido}-N-propyl-D-gluco-δ-lactam (Compound No. 79)
N-{4-[Phenyl]piperazinyl carboxamido}-D-gluco-δ-lactam (Compound No. 80)
2,3,4-Tri-O-benzyl-6-[4-chlorophenyl-3(2H,4H)-1,2,4-triazol-3-onyl]-N-allyl-D-gluco-δ-lactam (Compound No. 81)
N-Allyl-2,3,4-tri-O-benzyl-6-(2,6-diketopiperidino)-D-gluco-δ-lactam (Compound No. 82)
N-Allyl-2,3,4-tri-O-benzyl-6-(1H-isoindole-1,3(2H)-diketo)-D-gluco-δ-lactam (Compound No. 83)
N-Allyl-2,3,4-tri-O-benzyl-6-(2,5-diketopyrrolidino)-D-gluco-δ-lactam (Compound No. 84)
N-Allyl-2,3,4-tri-O-benzyl-1,5-dideoxy-1,5-imino-6-morpholino glucitol (Compound No. 85)
6-(2,5-Diketopyrrolidino)-N-propyl-D-gluco-δ-actam (Compound No. 86)
6-(2,6-Diketopiperidino)-N-propyl-D-gluco-δ-lactam (Compound No. 87)
N-Propyl-6-[4-chlorophenyl-3(2H,4H)-1,2,4-triazol-3-onyl]-D-gluco-δ-lactam (Compound No. 88)
2,3,4-Tri-O-benzyl-6-(4,6-dichloro-1,3,5-triazin-1-yl)-N-propyl-D-gluco-δ-lactam (Compound No. 89)
2,3,4-Tri-O-benzyl-6-O-(4,6-dichloro-1,3,5-triazin-1-yl)-N-propyl-D-gluco-δ-lactam (Compound No. 90)
   Pharmaceutical Compositions

The invention also provides for pharmaceutical compositions including a therapeutically effective amount of a compound of Formula I, or its pharmaceutically acceptable salts, pharmaceutically acceptable solvates, esters, enantiomers, diastereomers, N-oxides, polymorphs, prodrugs or metabolites, as described herein, along with a pharmaceutically acceptable carrier, and optionally but desirably, pharmaceutically acceptable excipients.

Preparations for parenteral administration of the pharmaceutical compositions described herein include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulation of the pharmaceutical compositions may be carried out in conventional manner using one or more physiologically and/or pharmaceutically acceptable carriers or excipients. Thus, the compounds and their pharmaceutically acceptable salts and solvates may be formulated for administration by inhalation or insufflation (either through the mouth or the nose) or oral, buccal, parenteral, or rectal administration. For oral administration, the pharmaceutical compositions may take the form of, for example, tablets or capsules prepared by conventional means with pharmaceutically acceptable excipients such as binding agents (for example, pregelatinized maize starch, polyvinylpyrrolidone, or hydroxypropyl methylcellulose); fillers (for example, lactose, microcrystalline cellulose or calcium hydrogen phosphate); lubricants (for example, magnesium stearate, talc or silica); disintegrants (for example, potato starch or sodium starch glycolate); or wetting agents (for example, sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (for example, sorbitol syrup, cellulose derivatives or hydrogenated edible fats); emulsifying agents (for example, lecithin or acacia); non-aqueous vehchles (for example, almond oil, oily esters, ethyl alcohol or fractionated vegetable oils); and preservatives (for example, methyl or propyl-p-hdroxybenzoates or sorbic acid). The preparations may also contain buffer salts, flavoring, coloring and sweetening agents as appropriate. Preparations for oral administration may be suitably formulated to give controlled release of the active compound.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol the dosage unit may be determined by providing a valve to deliver a metered amount. Capsules and cartridges of, for example, gelatin for use in an inhaler or insufflator may be formulated containing a powder mix of the compound and a suitable powder base such as lactose or starch.

The compounds may be formulated for parenteral administration by injection, for example, by bolus injection or continuous infusion. Formulations for injection may be presented in unit dosage form, for example, in ampoules or in multi-dose containers, with an added preservative. The compositions may take such forms as suspension, solutions or emulsions in oily or aqueous vehicles, and may contain formulatory agents such as suspending, stabilizing and/or dispersing agents. Alternatively, the active ingredient may be in powder form for constitution with a suitable vehicle, for example, sterile pyrogen-free water, before use.

The compounds may also be formulated in rectal compositions such as suppositories or retention enemas, for example, containing conventional suppository bases such as cocoa butter or other glycerides.

In addition to the formulations described previously, the compounds may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

The therapeutic compositions of the invention also contain a carrier or excipient, many of which are known to skilled artisans. Excipients which can be used include buffers (for example, citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. Methods for making such formulations are well-known and can be found in, for example, “Remington's Pharmaceutical Sciences.”

By “therapeutically effective amount” is meant the quantity of a compound or composition according to the invention necessary to prevent, cure or at least partially arrest the symptoms of the disorder and its complications. Amounts effective to achieve this goal will, of course, depend on the severity of the disease and the weight and general state of the patient. Typically, dosages used in vitro may provide useful guidance in the amounts useful for in situ administration of the pharmaceutical composition, and animal models may be used to determine effective dosages for treatment of particular disorders. Various considerations are described, for example, in Gilman et al., eds., 1900, “Goodman and Gilman's: The Pharmaceutical Bases of Therapeutics,” 8^{th} ed., Pergamon Press; and Remington's Pharmaceutical Sciences,” 1990, 17^{th} ed., Mack Publishing Co., Easton, Pa., each of which is hereby incorporated by reference.

Methods of Treating Cancer

The invention also provides for methods of treating cancer in a mammal. The methods include administering to a mammal a therapeutically effective amount of a compound having the structure of Formula I, or its pharmaceutically acceptable salts, pharmaceutically acceptable solvates, esters, enantiomers, diastereomers, N-oxides, polymorphs, prodrugs or metabolites, as described herein.

The invention also provides a method of inhibiting the metastasis of cancer cells in a mammal, including the administration of a therapeutically effective amount of a compound having the structure of Formula I, or its pharmaceutically acceptable salts, pharmaceutically acceptable solvates, esters, enantiomers, diastereomers, N-oxides, polymorphs, prodrugs or metabolites, as described herein.

The administration of pharmaceutical compositions can be by injection or by gradual infusion over time. The compositions can be administered intravenously, intraperitoneally, intramuscularly, subcutaneously, intracavity, or transdermally. Preferred methods for delivery of the compositions include orally, by encapsulation in microspheres or proteinoids, by aerosol delivery to the lungs, or transdermally by iontophoresis or transdermal electroporation. Other methods of administration will be known to those skilled in the art.

The invention will be further described in the following examples, which demonstrate general synthetic procedures, as well as specific preparations of some preferred compounds. The examples do not limit the scope of the invention described in the claims.

### EXAMPLES

The examples mentioned below demonstrate the general synthetic procedure as well as the specific preparation of the particular compounds. Particular properties of the compounds disclosed herein are also exemplified. The examples are given to illustrate the details of the invention and should not be construed to limit the scope of the present invention.

### Example 1

### Preparation of N-allyl-6-O-(D-toluenesulphonyl-2,3,4-tri-O-benzyl-D-gluco-δ8-lactam (Compound No. 1)

N-Allyl-2,3,4-tri-O-benzyl-D-gluco-δ-lactam (0.487 gm) (prepared by the method reported in Tetrahedron, 50 (14), 4215-4224 (1994), Tetrahedron Letters, 37 (4), 547-50 (1996) was dissolved in dichloromethane (10 ml). To this was added triethylamine (1.01 gm) and cooled it to 0° C. Added p-toluene sulphonyl chloride (1.141 gm) to the reaction mixture and concentrated and stirred for additional 2 hours. The contents of the reaction mixture were poured into ice cold water (50 ml). The compound was extracted with dichloromethane (2 times, 25 ml). The organic layer was dried over anhydrous sodium sulphate (Na₂SO₄) and the solvent was evaporated under vacuum. The crude material was purified by column chromatography using ethylacetate-hexane (2:8) as eluent mixture to yield the title compound in a semi solid state.

The following spectral information was used to confirm product formation: IR (CH₂Cl₂): ν 1674 cm⁻; ¹HNMR (CDCl₃): δ 2.41 (s, 3H, CH₃), 3.40-3.48 (dd, J=8 Hz, 15 Hz, 1H, CH), 3.62-3.65 (m, 1H, CH), 3.71-3.75 (t, J=6 Hz, 1H, CH), 3.78-3.82 (t, J=6 Hz, 1H, CH), 3.88-3.91 (m, 1H, CH), 4.03-4.16 (m, 2H, 2×CH), 0.43-4.39 (d, J=12 Hz, 1H, PhCH), 4.45-4.51 (m, 1H, CH), 4.55-4.59 (d, J=12 Hz, 1H, PhCH), 4.62-4.66 (m, 3H, 2×PhCH+CH), 4.71-4.75 (d, J=12 Hz, 1H, PhCH), 5.08-5.15 (m, 3H, 2×CH+PhCH), 5.60-5.70 (m, 1H, CH), 7.16-7.33 (m, 15H, ArH), 7.39-7.49 (d, J=6 Hz, 2H, ArH), 7.71-7.73 (d, J=6 Hz, 2H, ArH); Mass: m/z 641.1 (M⁺+1).

### Example 2

### Preparation of 2,3-4-tri-O-benzyl-6-N-{[2-naphthyl]aminothiocarbonylamino}N-propyl-D-gluco-δ-lactam (Compound No.2)

Step 1: Preparation of 6-Allyl-6-O-(p-toluene sulphonyl)-2,3,4-tri-O-benzyl-D-gluco-δ-lactam.

The title compound was prepared as described in Example 1.

Step 2: Preparation of 6-Azido-N-allyl-2,3,4-tri-O-benzyl-D-gluco-δ-lactam

The product obtained from Step 1 (0.641 gm) was dissolved in N,N-dimethyl formamide (DMF) (10 ml). To this was added sodium azide (NaN₃) (0.390 gm), ammonium chloride (NH₄Cl) (catalytic amount) and the reaction mixture was stirred at 40° C. for 5 hours. The reaction mixture was poured into cold water (50 ml). The product was extracted with ethylacetate (2 times, 25 ml) and the organic layer was washed with brine (50 ml). The organic layer was dried over anhydrous Na₂SO₄ and the solvent was distilled off under reduced pressure. The compound was purified using column chromatography with ethylacetate-hexane mixture as eluent.

Step 3: Preparation of 6-Amino-N-propyl-2,3,4-tri-O-benzyl-D-gluco-δ-lactam

The above compound obtained in Step 2 (0.512 gm) was dissolved in ethanol (10 ml) and palladium/carbon (Pd/c) (10%, 0.102 gm) was added and the system was made under hydrogen using hydrogen balloon. The reaction was stirred for 5 hours. The reaction mixture was then filtered trough celite, washed with methanol (50 ml) and the solvent removed under vacuum. The compound obtained was used as such for the next step without further purification.

Step 4: Preparation of 2,3,4-Tri-O-benzyl-6-N-[2-naphthyl]aminothiocarbonylamino}-N-propyl-D-gluco-δ-lactam

6-Amino-N-propyl-2,3,4-tri-O-benzyl-D-gluco-δ-lactam prepared as described in Step 3, (0.488 gm, 1 mmol) was dissolved in acetonitrile (10 ml). To this was added 2-naphthyl isothiocyanate (1.2 mmol) and stirred the reaction mixture for 2 hours at room temperature. Solvent was distilled off under vacuo and the compound was purified by column chromatography using ethylacetate-Hexane (4:6) as the eluent mixture to yield the title compound in the semisolid state.

The following spectral information was used to confirm product formation: IR (CH₂Cl₂): ν 1643.5 cm⁻¹; ¹HNMR (CDCl₃): δ 0.85-0.90 (t, J=7 Hz, 3H, CH₃), 1.55 (m, 2H, CH₂), 3.05-3.07 (m, 1H, CH), 3.36-3.41 (m, 2H, 2×CH), 3.54-3.56 (m, 1H, CH), 3.69-3.79 (m, 2H, 2×CH), 4.04-4.15 (m, 3H, 2×CH+PhCH), 4.23-4.27 (d, J=12 Hz, 1H, PhCH), 4.45 (m, 2H, PhCH), 4.54-4.58 (d, J=12 Hz, 1H, PhCH), 4.98-5.02 (d, J=12 Hz, 1H, PhCH), 5.84 (m, 1H, NH), 6.89-7.94 (m, 22H, ArH); Mass: m/z 673 (M⁺+1).

The following compounds were prepared analogously:

### Example 2A

### Preparation of 2,3,4-Tri-O-benzyl-6-N-{[4-chlorophenyl]aminocarbonylamino}-N-propyl-D-gluco-δ-lactam (Compound No. 3)

The title compound was prepared by using 4-chlorophenyl isocyanate in place of 2-naphthyl isothiocyanate in the above Example 2 to yield the title compound in semisolid state. The following spectral information was used to confirm product formation: IR (CH₂Cl₂): ν 1672, 1647 cm⁻¹; ¹HNMR (CDCl₃): δ 0.84-0.89 (t, J=7 Hz, 3H, CH₃), 1.52-1.62 (m, 2H, CH₂), 2.89-2.96 (m, 1H, NCH), 3.28-3.33 (m, 1H, NH), 3.55-3.57 (m, 1H, CH), 3.69-3.88 (m, 4H, 4×CH), 4.03-4.05 (d, J=7 Hz, 1H, CH), 4.55-4.59 (d, J=12 Hz, 1H, PhCH), 4.63-4.71 (m, 4H, 4×PhCH), 4.90-4.94 (d, J=12 Hz, 1H, PhCH), 5.47-5.51 (m, 1H, NH), 7.08-7.34 (m, 19H, ArH); Mass: m/z 642 (M⁺+1).

### Example 2B

### Preparation of 2,3,4-Tri-O-benzyl-6-N-{[p-methoxyphenyl]aminocarbonylamino}-N-propyl-D-gluco-δ-lactam (Compound No. 4)

The title compound was synthesised by using 4-methoxyphenyl isocyanate instead of 2-naphthyl isothiocyanate in Example 2 to give the title compound in semisolid state. The following spectral information was used to confirm product formation: IR (CH₂Cl₂): ν 1646 cm⁻¹; ¹HNMR (CDCl₃): δ 0.86-0.91 (t, J=7 Hz, 3H, CH₃), 1.52-1.62 (m, 2H, CH₂), 2.99 (m, 1H, NCH), 3.18 (m, 1H, NCH), 3.55-3.65 (m, 2H, 2×CH), 3.71 (m, 5H, OCH₃+2×CH), 3.75-3.86 (m, 1H, CH), 3.98-4.01 (d, J=9 Hz, 1H, CH), 4.45-4.49 (d, J=12 Hz, 1H, PhCH), 4.56-4.65 (m, 4H, 4×PhCH), 4.93 (brm, 1H, NH), 5.04-5.08 (d, J=12 Hz, 1H, PhCH), 6.34 (s, 1H, NH), 6.77-6.80 (d, J=9 Hz, 2H, ArH), 7.05-7.08 (d, J=9 Hz, 2H, ArH), 7.15-7.39 (m, 15H, ArH); Mass: m/z 638 (M⁺+1).

### Example 2C

### Preparation of 2,3,4-Tri-O-benzyl-6-N-{[p-nitrophenyl]aminocarbonylamino}-N-propyl-D-gluco-δ-lactam (Compound No. 5)

The title compound was prepared by using 4-nitrophenyl isocyanate instead of 2-napthyl isothiocyanate in Example 2 to give the title compound in semisolid state. The following spectral information was used to confirm product formation: IR (CH₂C1₂): ν 1707, 1644, 1504, 1329 cm⁻¹; ¹HNMR (CDCl₃): δ 0.84-0.89 (t, J=7 Hz, 3H, CH₃), 1.54-1.63 (m, 2H, CH₂), 2.86-2.90 (m, 1H, NCH), 3.47-3.59 (m, 1H, NCH), 3.62-3.72 (m, 1H, CH), 3.74-3.79 (m, 1H, CH), 3.83-3.95 (m, 3H, 3×CH), 4.07-4.10 (d, J=9 Hz, 1H, CH), 4.59-4.63 (d, J=12 Hz, 1H, PhCH), 4.67-4.77 (m, 5H, 5×PhCH), 6.14 (brt, 1H, NH), 7.09-7.34 (m, 15H, ArH), 7.45-7.48 (d, J=9 Hz, 2H, ArH), 8.02-8.05 (d, J=9 Hz, 2H, ArH), 8.24 (s, 1H, NH); Mass: m/z 653 (M⁺+1).

### Example 2D

### Preparation of 2,3,4-Tri-O-benzyl-6-N-{[p-tolyl]aminocarbonylamino}-N-propyl-D-gluco-δ-lactam (Compound No. 6)

The title compound was obtained by using 4-tolyl isocyanate instead of 2-napthyl isothiocyanate in Example 2 to give the title compound in semisolid state. The following spectral information was used to confirm product formation: IR (CH₂Cl₂): ν 1672, 1645 cm⁻¹; ¹HNMR (CDCl₃): δ 0.86-0.90 (t, J=7 Hz, 3H, CH₃), 1.55-1.61 (m, 2H, CH₂), 2.29 (s, 3H, CH₃), 2.98-3.00 (m, 1H, NCH), 3.19-3.22 (m, 1H, NCH), 3.56-3.61 (m, 2H, 2×CH), 3.65-3.66 (m, 2H, 2×CH), 3.83-3.87 (m, 1H, CH), 3.99-4.01 (d, J=7 Hz, 1H, CH), 4.46-4.49 (d, J=12 Hz, 1H, PhCH), 4.56 (m, 2H, 2×PhCH), 4.66-4.69 (m, 2H, 2×PhCH), 4.97-4.99 (brt, 1H, NH), 5.04-5.08 (d, J=12 Hz, 1H, PhCH), 6.32 (s, 1H, NH), 7.06-7.39 (m, 19H, ArH); Mass: m/z 622 (M⁺+1).

### Example 2E

### Preparation of 2,3,4-Tri-O-benzyl-6-N-{[4-chloro-2-trifluoromethylphenyl]aminocarbonylamino}-N-propyl-D-gluco-δ-lactam (Compound No. 7)

The title compound was synthesized by using 4-chloro-2-trifluoromethyl phenyl isocyanate instead of 2-naphthyl isothiocyanate in Example 2 to give the title compound in semisolid state. The following spectral information was used to confirm product formation: IR (CH₂Cl₂): ν 1702, 1651 cm⁻¹; ¹HNMR (CDCl₃): δ 0.83-0.89 (t, J=7 Hz, 3H, CH₃), 1.54-1.63 (m, 2H, CH₂), 2.96-3.00 (m, 1H, NCH), 3.27-3.20 (m, 1H, CH), 3.51-3.56 (m, 1H, CH), 3.64-3.66 (m, 2H, 2×CH), 3.63-3.80 (m, 1H, CH), 3.87-3.90 (m, 1H, CH), 4.03-4.05 (d, J=7 Hz, 1H, CH), 4.49-4.53 (d, J=12 Hz, 1H, PhCH), 4.57 (s, 2H, 2×PhCH), 4.66-4.72 (m, 2H, PhCH), 5.03-5.07 (d, J=12 Hz, 1H, PhCH), 5.25 (brt, 1H, NH), 6.52 (s, 1H, NH), 7.19-7.85 (m, 18H, ArH); Mass: m/z 710 (M⁺+1).

### Example 2F

### Preparation of 2,3,4-Tri-O-benzyl-6-N-{[isopropyl]aminothiocarbonylamino}-N-propyl-D-gluco-δ-lactam (Compound No. 8)

The title compound was obtained by taking isopropyl isothiocyanate in place of 2-naphthyl isothiocyanate in Example 2 in a semisold state. The following spectral information was used to confirm product formation: IR (CH₂Cl₂): ν 1649 cm⁻¹; ¹HNMR (CDCl₃): δ 0.86-0.93 (t, J=3 Hz, 3H, CH₃), 1.03-1.07 (t, J=6 Hz, 6H, 2×CH₃), 1.62 (m, 2H, CH₂), 3.08-3.12 (m, 1H, NCH), 3.46 (m, 1H, NCH), 3.61-3.74 (m, 3H, 3×CH), 3.89-3.95 (m, 1H, CH), 4.05-4.15 (m, 3H, 2×CH+CH), 4.43-4.47 (d, J=12 Hz, 1H, PhCH), 4.56-4.67 (m, 4H, 4×PhCH), 4.70-4.74 (d, J=12 Hz, 1H, PhCH), 5.95 (brs, 1H, NH), 7.17-7.19 (m, 2H, ArH), 7.26-7.31 (m, 11H, ArH), 7.39-7.41 (m, 2H, ArH); Mass: m/z 590 (M⁺+1).

### Example2G

### 2,3,4-Tri-O-benzyl-6-N-{[phenyl]aminothiocarbonylamino}-N-propyl-D-gluco-δ-lactam (Compound No. 9)

The title compound was prepared by using phenyl isothiocyanate instead of 2-naphthyl isothiocyanate in Example 2 to give the title compound in semisolid state. The following spectral information was used to confirm product formation: IR (CH₂Cl₂): ν 1671, 1649 cm⁻¹; ¹HNMR (CDCl₃): δ 0.89-0.93 (t, J=7 Hz, 3H, CH₃), 1.62 (m, 2H, CH₂), 3.11-3.14 (m, 1H, NCH), 3.46-3.50 (m, 2H, 2×CH), 3.66 (m, 1H, CH), 3.80-3.82 (m, 1H, CH), 3.96-3.98 (m, 1H, CH), 4.08-4.14 (m, 2H, 2×CH), 4.32-4.36 (d, J=12 Hz, 1H, PhCH), 4.44-4.52 (m, 3H, 3×PhCH), 4.64-4.68 (d, J=12 Hz, 1H, PhCH), 5.05-5.09 (d, J=12 Hz, 1H, PhCH), 6.17 (brs, 1H, NH), 6.98-7.05 (m, 4H, ArH), 7.18-7.40 (m, 16H, ArH), 7.87 (s, 1H, NH); Mass: m/z 624 (M⁺+1).

### Example 2H

### Preparation of 2,3,4-Tri-O-benzyl-6-N-{[phenyl]aminocarbonylamino}-N-propyl-D-gluco-δ-lactam (Compound No. 10)

The title compound was obtained by using phenyl isocyanate in place of 2-naphthyl isothiocyanate in Example 2 to give the title compound in semisolid state. The following spectral data was used to confirm product formation: IR (CH₂Cl₂): ν 1671, 1648 cm⁻¹; ¹HNMR (CDCl₃): δ 0.84-0.89 (t, J=3 Hz, 3H, CH₃), 1.55-1.61 (m, 2H, CH₂), 2.93-2.95 (m, 1H, NCH), 3.42-3.49 (m, 1H, NCH), 3.70-3.74 (m, 2H, 2×CH), 3.84-3.88 (m, 3H, 3×CH), 4.02-4.05 (d, J=7 Hz, 1H, CH), 4.51-4.55 (d, J=12 Hz, 1H, PhCH), 4.60-4.69 (m, 4H, 4×PhCH), 4.96-5.00 (d, 1H, J=12 Hz, 1H, PhCH), 5.55 (m, 1H, NH), 6.97-7.33 (m, 20H, ArH); Mass: m/z 608 (M⁺+1).

### Example 2I

### Preparation of 2,3,4-Tri-O-benzyl-6-N-{[p-fluorophenyl]aminocarbonylamino}-N-propyl-D-gluco-δ-lactam (Compound No. 11)

The title compound was obtained by reacting 4-fluorophenyl isocyanate instead of 2-naphthyl isothiocyanate in Example 2 to give the title compound in semisolid state. The following spectral information was used to confirm product formation: IR (CH₂Cl₂): ν 1670, 1647 cm⁻¹; ¹HNMR (CDCl₃): δ 0.85-0.90 (t, J=7 Hz, 3H, CH₃), 1.58-1.60 (m, 2H, CH₂), 2.93 (m, 1H, CH), 2.97 (m, 1H, CH), 3.69 (m, 1H, CH), 3.70 (m, 1H, CH), 3.85-3.88 (m, 3H, 3×CH), 4.05-4.02 (d, J=7 Hz, 1H, CH), 4.52-4.56 (d, J=12 Hz, 1H, PhCH), 4.66-4.70 (m, 4H, 4×PhCH), 4.99-4.95 (d, J=12 Hz, 1H, PhCH) 5.20 (m, 1H, NH), 6.87-6.94 (m, 2H, ArH), 7.16-7.33 (m, 17H, ArH); Mass: m/z 626 (M⁺+1).

### Example 2J

### Preparation of 2,3,4-Tri-O-benzyl-6-N-{[p-chlorophenylsulfonyl]aminocarbonylamino}-N-propyl-D-gluco-δ-lactam (Compound No. 12)

The title compound was prepared by using 4-chlorophenylsulfonyl isocyanate instead of 2-naphthyl isothiocyanate in Example 2, in a semisolid state. The following spectral data was used to confirm product formation: IR (CH₂Cl₂): ν 1671, 1648 cm⁻¹; ¹HNMR (CDCl₃): δ 0.80-0.85 (t, J=7 Hz, 3H, CH₃), 1.51-1.55 (m, 2H, CH₂), 2.25 (m, 1H, CH), 2.93-2.97 (m, 1H, NCH), 3.42 (m, 1H, NCH), 3.59-3.75 (m, 3H, 3×CH), 3.88-3.90 (m, 1H, CH), 4.09-4.11 (d, J=7 Hz, CH), 4.53-5.57 (m, 3H, 3×PhCH), 4.64-4.69 (d, J=12 Hz, 1H, PhCH), 4.96-4.99 (d, J=12 Hz, 1H, PhCH), 6.67 (m, 1H, NH), 7.07-7.37 (m, 17H, ArH), 7.73-7.76 (d, J=9 Hz, 2H, ArH), 8.52 (brs, 1H, NH); Mass: m/z 706 (M⁺+1).

### Example 2K

### Preparation of 2,3,4-Tri-O-benzyl-6-N-{[3-chlorophenyl]aminocarbonylamino}-N-propyl-D-gluco-δ-lactam (Compound No. 13)

The title compound was prepared by using 3-chlorophenylsiocyanate instead of 2-naphthyl isothiocyante in Example 2 in a semisolid state. The following spectral data was used to confirm product formation: IR (CH₂Cl₂): ν 1671, 1648 cm⁻¹; ¹HNMR (CDCl₃): δ 0.84-0.89 (t, J=7 Hz, 3H, CH₃), 1.55-1.60 (m, 2H, CH₂), 2.91-2.93 (m, 1H, NCH), 3.36 (m, 1H, NCH), 3.54 (m, 1H, CH), 3.73-3.89 (m, 4H, 4×CH), 4.04-4.07 (d, J=9 Hz, 1H, CH), 4.60-4.73 (m, 5H, 5×PhCH), 4.84-4.88 (d, J=12 Hz, 1H, PhCH), 5.76 (brs, 1H, NH), 6.93-6.96 (m, 1H, NH), 7.09-7.42 (m, 19H, ArH); Mass: m/z 642 (M⁺+1).

### Example 2L

### Preparation of 2,3,4-Tri-O-benzyl-6-N-{[3-acetylphenyl]aminocarbonylamino}-N-propyl-D-gluco-δ-lactam (Compound No. 14)

The title compound was synthesised by reacting 3-acetyl phenyl isocyanate with the amine instead of 2-naphthyl isothiocyanate in Example 2 to give the title compound in a semisolid state. The following spectral information was used to confirm product formation: IR (CH₂Cl₂): ν 1670, 1655, 1650 cm⁻¹; ¹HNMR (CDCl₃): δ 0.84-0.89 (t, J=3 Hz, 3H, CH₃), 1.56-1.65 (m, 2H, CH₂), 2.59 (s, 3H, COCH₃), 3.36 (m, 1H, NCH), 3.56 (m, 1H, CH), 3.73-3.90 (m, 4H, 4×CH), 4.08-4.10 (d, J=7 Hz, 1H, CH), 4.57-4.61 (d, J=2 Hz, 1H, PhCH), 4.63-4.68 (m, 3H, 3×PhCH), 4.69-4.73 (d, J=12 Hz, 1H, PhCH) 4.93-4.97 (d, J=12 Hz, 1H, CH), 5.80 (s, 1H, NH), 7.19-7.77 (m, 19H, ArH); Mass: m/z 650 (M⁺+1).

### Example 2M

### Preparation of 2,3,4-Tri-O-benzyl-6-N-{[3-chloro-6-methoxyphenyl]aminocarbonylamino}-N-propyl-D-gluco-δ-lactam (Compound No. 15)

The title compound was synthesised by reacting 3-chloro-6-methoxyphenyl isocyanate with the amine in place of 2-naphthyl isothiocyanate in Example 2 to yield the title compound in a semisolid state. The following spectral information was used to confirm product formation: IR (CH₂Cl₂): ν 1671, 1648 cm⁻¹; ¹HNMR (CDCl₃): δ 0.86-0.91 (t, J=7 Hz, 3H, CH₃), 1.57-1.62 (m, 2H, CH₂), 2.98-3.00 (m, 1H, NCH), 3.25 (m, 1H, NCH), 3.69-3.74 (m, 2H, 2×CH), 3.78-3.80 (m, 5H, 2×CH+OCH₃), 4.03 (m, 1H, CH), 4.03-4.06 (d, J=9 Hz, 1H, CH), 4.53-4.57 (d, J=12 Hz, 1H, PhCH) 4.59-4.68 (m, 3H, 3×PhCH), 4.72-4.76 (d, J=12 Hz, 1H, PhCH), 5.07-5.11 (d, J=12 Hz, 1H, PhCH), 6.72-6.75 (d, J=9 Hz, 1H, ArH), 6.91-6.93 (m, 2H, NH+ArH), 7.21-7.40 (m, 15H, ArH), 8.14-8.15 (m, 1H, ArH); Mass: m/z 672 (M⁺+1).

### Example 2N

### Preparation of 2,3,4-Tri-O-benzyl-6-N-{[2,4-difluorophenyl]aminocarbonylamino}-N-propyl-D-gluco-δ-lactam (Compound No. 16)

The title compound was synthesised by using 2,4-difluorophenyl isocyanate instead of 2-naphthyl isothiocyanate in Example 2 in a semisolid state. The following spectral information was used to confirm product formation: IR (CH₂Cl₂): ν 1701, 1645 cm⁻¹; ¹HNMR (CDCl₃): δ 0.84-0.89 (m, 3H, CH₃), 1.57-1.59 (m, 2H, CH₂), 2.96 (m, 1H, NCH), 3.28-3.30 (m, 1H, NCH), 3.52-3.57 (m, 1H, CH), 3.70-3.90 (m, 4H, 4×CH), 4.06-4.08 (d, J=7 Hz, 1H, CH), 4.54-4.84 (m, 5H, 5×PhCH), 4.95-4.99 (d, J=12 Hz, 1H, PhCH), 5.67 (brs, 1H, NH), 6.75-6.82 (m, 2H, ArH), 7.21-7.29 (m, 15H, ArH). 7.93-7.94 (m, 1H, ArH); Mass: m/z 644 (M⁺+1).

### Example 2P

### Preparation of 2,3,4-Tri-O-benzyl-6-N-{[2,4,6-trichlorophenyl]aminocarbonylamino}-N-propyl-D-gluco-δ-lactam (Compound No. 17)

The title compound was prepared by using 2,4,6-trichlorophenylisocyanate in place of 2-naphthyl isothiocyanate Example 2 to give the title compound in semisolid state. The following spectral information was used to confirm product formation: IR (CH₂Cl₂): ν 1646, 1670 cm⁻¹; ¹HNMR (CDCl₃): δ 0.85-0.90 (t, J=7 Hz, 3H, CH₃), 1.57-1.59 (m, 2H, CH₂), 2.96-3.00 (m, 1H, CH), 3.33 (m, 1H, CH), 3.53-3.58 (m, 1H, CH), 3.74-3.84 (m, 3H, 3×CH), 4.06-4.09 (d, J=7 Hz, 1H, CH), 4.49-4.53 (d, J=12 Hz, 1H, PhCH), 4.59-4.63 (m, 3H, 3×PhCH), 4.67-4.71 (d, J=12 Hz, 1H, PhCH), 4.98-5.02 (d, J=12 Hz, 1H, PhCH), 5.50-5.51 (m, 1H, NH), 6.51 (m, 1H, NH), 7.16-7.40 (m, 17H, ArH); Mass: m/z 710 (M⁺+1).

### Example 2Q

### Preparation of 2,3,4-Tri-O-benzyl-6-N-{[3,4-dichlorophenyl]aminocarbonylamino}-N-propyl-D-gluco-δ-lactam (Compound No. 18)

The title compound was synthesized by taking 3,4-dichlorophenylisocyanate instead of 2-naphthyl isothiocyanate Example 2 to give the title compound in semisolid state. The following spectral information was used to confirm product formation: IR (CH₂Cl₂): ν 1701, 1646 cm⁻¹; ¹HNMR (CDCl₃): δ 0.85-0.89 (t, J=7 Hz, 3H, CH₃), 1.62 (m, 2H, CH₂), 2.90 (m, 1H, NCH), 3.39 (m, 1H, CH), 3.58-3.59 (m, 1H, CH), 3.72-3.75 (m, 2H, 2×CH), 3.85-3.90 (m, 2H, 2×CH), 4.03-4.06 (d, J=7 Hz, 1H, CH), 4.58-4.62 (d, J=12 Hz, 1H, PhCH), 4.64-4.81 (m, 5H, 5×PhCH), 5.91 (s, 1H, NH), 7.15-7.52 (m, 18H, ArH), 7.67 (s, 1H, NH); Mass: m/z 676 (M⁺+1).

### Example 2R

### Preparation of 2,3,4-Tri-O-benzyl-6-N-{[2,4-dichlorophenyl]aminocarbonylamino}-N-prropyl-D-gluco-δ-lactam (Compound No. 19)

The title compound was prepared by using 2,4-dichlorophenylisocyanate instead of 2-naphthyl isothiocyanate in Example 2 to give the title compound in semisolid state. The following spectral information was used to confirm product formation: IR (CH₂Cl₂): ν 1701, 1648 cm⁻¹; ¹HNMR (CDCl₃): δ 0.85-0.90 (t, J=7 Hz, 3H, CH₃), 1.55-1.63 (m, 2H, CH₂), 2.92-3.01 (m, 1H, NCH), 3.27-3.31 (m, 1H, NCH), 3.53-3.58 (m, 1H, CH), 3.67-3.90 (m, 4H, 4×CH), 4.05-4.07 (d, J=7 Hz, 1H, CH), 4.53-4.73 (m, 5H, 5×PhCH), 5.00 (d, J=9 Hz, 1H, PhCH), 5.65 (brs, 1H, NH), 6.95 (brs, 1H, NH), 7.16-7.35 (m, 17H, ArH), 8.03-8.06 (d, J=9 Hz, 1H, ArH), 8.03-8.06 (d, J=9 Hz, 1H, ArH); Mass: m/z 676 (M⁺+1).

### Example 2S

### Preparation of 2,3,4-Tri-O-benzyl-6-N-{[isopropyl]aminocarbonylamino}-N-propyl-D-gluco-δ-lactam (Compound No. 20)

The title compound was obtained by using isopropyl isocyanate in place of 2-naphthyl isothiocyanate in Example 2 to give the title compound in semisolid state. The following spectral information was used to confirm product formation: IR (CH₂Cl₂): ν 1655, 1560 cm⁻¹; ¹HNMR (CDCl₃): δ 0.87-0.92 (t, J=7 Hz, 3H, CH₃), 1.05-1.15 (m, 6H, 2×CH₃), 1.56-1.63 (m, 2H, CH₂), 2.98-3.02 (m, 1H, CH), 3.11-3.16 (m, 1H, CH), 3.51-3.58 (m, 1H, CH), 3.65-3.74 (m, 4H, 4×CH), 3.87-3.89 (m, 1H, CH), 4.01-4.04 (d, J=7 Hz, 1H, CH), 4.50-4.54 (d, J=12 Hz, 1H, PhCH), 4.58 (m, 2H, 2×PhCH), 4.69-474 (m, 2H, 2×PhCH), 5.08-5.12 (d, J=12 Hz, 1H, PhCH), 7.13-7.43 (m, 15H, ArH); Mass: m/z 574 (M⁺+1).

### Example 2T

### Preparation of 2,3,4-Tri-O-benzyl-6-N-{[4-chlorophenyl]aminothiocarbonylamino}-N-propyl-D-gluco-δ-lactam (Compound No. 21)

The title compound was obtained by taking 4-chlorophenyl isothiocyanate instead of 2-naphthyl isothiocyanate in Example 2 to give the title compound in semisolid state. The following spectral information was used to confirm product formation: IR (CH₂Cl₂): ν 1648, 1536, 1492 cm⁻¹; ¹HNMR (CDCl₃): δ 0.89-0.94 (t, J=7 Hz, 3H, CH₃), 1.57-1.63 (m, 2H, CH₂), 3.07-3.14 (m, 1H, NCH), 3.51 (m, 2H, 2×CH), 3.68-3.85 (m, 2H, 2×CH), 3.80-3.99 (m, 3H, 3×CH), 4.33-4.37 (d, J=12 Hz, 1H, PhCH), 4.46-4.50 (d, J=12 Hz, 1H, PhCH), 4.46-4.50 (d, J=12 Hz, 1H, PhCH), 4.55 (m, 2H, 2×PhCH), 4.68-4.72 (d, J=12 Hz, 1H, PhCH), 5.04-5.08 (d, J=12 Hz, 1H, PhCH), 6.89-7.39 (m, 19H, ArH); Mass: m/z 658 (M⁺+1).

### Example 2U

### Preparation of 2,3,4-Tri-O-benzyl-6-N-{[4-trifluoromethylphenyl]aminocarbonylamino}-N-propyl-D-gluco-δ-lactam (Compound No. 22)

The title compound was synthesized by taking 4-trifluoromethylphenyl isocyanate in place of 2-naphthyl isothiocyanate in Example 2 to give the title compound in semisolid state. The following spectral information was used to confirm product formation: ¹HNMR (CDCl₃): δ 0.83-0.88 (t, J=7 Hz, 3H, CH₃), 1.55-1.60 (m, 2H, CH₂), 2.90 (m, 1H, CH), 3.41 (m, 1H, CH), 3.57 (m, 1H, CH), 3.74-3.85 (m, 2H, 2×CH), 3.88-3.92 (m, 2H, 2×CH), 4.07-4.09 (d, J=7 Hz, 1H, CH), 4.60-4.73 (m, 5H, 2×PhCH₂+PhCH), 4.81-4.85 (d, J=7 Hz, 1H, PhCH), 6.00 (m, 1H, NH), 7.15-7.46 (m, 19H, ArH), 7.88 (s, 1H, NH); Mass: m/z 676 (M⁺+1).

### Example 2V

### Preparation of 2,3,4-Tri-O-benzyl-6-N-{[1-adamantyl]aminocarbonylamino}-N-propyl-D-gluco-δ-lactam (Compound No. 23)

The title compound was synthesized by taking 1-adamantyl isocyanate in place of 2-naphthyl isothiocyanate in Example 2 to give the title compound in semisolid state. The following spectral information was used to confirm product formation: IR (DCM): δ 1646.7 cm⁻¹; ¹HNMR (CDCl₃): δ 0.87-0.91 (t, J=6 Hz, 3H, CH₃), 1.61-1.65 (m, 7H, 7×CH), 1.89 (s, 6H, 6×CH), 2.05 (s, 3H, 3×CH), 2.98-3.06 (m, 2H, 2×CH), 3.46-3.48 (m, 1H, CH), 3.66-3.76 (m, 3H, 3×CH), 3.84-3.88 (m, 1H, CH), 4.00-4.03 (d, J=9 Hz, 2H, 2×CH), 4.33-4.36 (m, 1H, NH), 4.50-4.75 (m, 5H, 5×PhCH), 5.08-5.12 (d, J=12 Hz, 1H, PhCH), 7.21-7.43 (m, 15H, ArH); Mass: m/z 666 (M⁺+1).

### Example 3

### Preparation of N-propyl-6-N-{[phenyl]aminocarbonylamino}-D-gluco-δ-lactam (Compound No. 24)

The ester as obtained in Example 2 (1 mmol) was dissolved in ethyl alcohol (5 ml). To this was added palladium/carbon (Pd/C) (0.641 mg, 10% dry) and cyclohexene (10 ml) and the reaction mixture was heated to reflux temperature. Stirring was done for another 12 hours at this temperature. The reaction mixture was then filtered through celite, washed with methanol and the mother liquor removed under vacuum. The residue obtained was purified by column chromatography using chloroform:methanol (8:2) as an eluent mixture. The product had m.p.: 173° C.

The following spectral information was used to confirm product formation: IR(CH₂Cl₂): ν 3342, 1672, 1648 cm⁻¹; ¹HNMR (CDCl₃): δ 0.81-0.86 (t, J=7 Hz, 3H, CH₃), 1.45-1.60 (m, 2H, CH₂), 2.95-3.04 (m, 1H, NCH), 3.51-3.77 (m, 6H, 6×CH), 3.99-4.02 (d, J=9 Hz, 1H), 7.12-7.39 (m, 5H, ArH); Mass: m/z 338 (M⁺+1).

The following compounds were prepared analogously, as will be appreciated by one of ordinary skill in the art:

### Example 3A

### Preparation of 6-N-{[2-Trifluoromethylphenyl]aminocarbonylamino}-N-propyl-D-gluco-δ-lactam (Compound No. 25)

The title compound was prepared, and exhibited an m.p.: 131° C. The following spectral information was used to confirm product formation: IR(KBr): ν1671, 1652 cm⁻¹; ¹HNMR (CDCl₃): δ 0.86-0.91 (t, J=7 Hz, 3H, CH₃), 1.50-1.65 (m, 2H, CH₂), 3.00-3.05 (m, 1H, NCH), 3.39-3.47 (m, 2H, 2×CH), 3.55-3.61 (t, J=9 Hz, 1H, CH), 3.73-3.88 (m, 4H, 4×CH), 3.91 (brs, 1H, OH), 4.09 (brs, 1H, NH), 4.86 (brs, 1H, OH), 7.08-7.13 (t, J=7 Hz, 1H, ArH), 7.45-7.54 (m, 2H, ArH), 7.70 (s, 1H, NH), 7.97-8.00 (d, J=9 Hz, 1H, ArH); Mass: m/z 406 (M⁺+1).

### Example 3B

### Preparation of 6-N-{[p-Tolyl]aminocarbonylamino}-N-propyl-D-gluco-δ-lactam (Compound No. 26)

The title compound was prepared, and exhibite an m.p.: 188° C. The following spectral information was used to confirm product formation: IR(KBr): ν 3327, 1671, 1648 cm⁻¹; ¹HNMR (CDCl₃): δ 0.88-0.93 (t, J=7 Hz, 3H, CH₃), 1.49-1.65 (m, 2H, CH₂), 2.26 (s, 3H, CH₃), 3.02-3.08 (m, 1H, CH), 3.35-3.39 (m, 2H, 2×CH), 3.55-3.59 (m, 1H, CH), 3.71-3.79 (m, 3H, 3×CH), 3.87-3.91 (m, 1H, CH), 4.19-4.20 (d, J=8 Hz, 1H, OH), 4.84-4.85 (d, J=9 Hz, 2H, ArH), 7.23-7.26 (d, J=9 Hz, 2H, ArH), 8.13 (s, 1H, NH); Mass: m/z 352 (M⁺+1).

### Example 3C

### Preparation of 6-N-{[p-Methoxyphenyl]aminocarbonylamino}-N-propyl-D-gluco-δ-lactam (Compound No.27)

The title compound was prepared, and exhibited an m.p.: 160° C. The following spectral information was used to confirm product formation: IR(KBr): ν 1671, 1649 cm⁻¹; ¹HNMR (CDCl₃): δ 0.87-0.92 (t, J=7 Hz, 3H, CH₃), 1.54-1.62 (m, 2H, CH₂), 3.10 (m, 1H, CH), 3.35 (m, 2H, 2×CH), 3.55 (m, 1H, CH), 3.75 (m, 6H, 3×CH+OCH₃), 3.89 (m, 1H, CH) 4.34 (brs, 1H, OH), 4.96 (brs, 1H, OH), 5.16 (brs, 1H, OH), 6.12 (brs, 1H, NH), 6.76-6.78 (d, J=9 Hz, 2H, ArH), 7.26-7.29 (d, J=9 Hz, ArH), 8.14 (brs, 1H, NH); Mass: m/z 368 (M⁺+1).

### Example 3D

### Preparation of 6-N-{[p-Aminophenyl]aminocarbonylamino}-N-propyl-D-gluco-δ-lactam (Compound No. 28)

The title compound was prepared. The following spectral information was used to confirm product formation: IR(KBr): ν 3403, 1670, 1647 cm⁻¹; ¹HNMR (CD₃)₂SO: δ 0.85-0.90 (t, J=7 Hz, 3H, CH₃), 1.52-1.59 (m, 2H, 2×CH), 2.99-3.07 (m, 2H, 2×CH), 3.43-3.93 (m, 6H, 6×CH), 4.49 (m, 1H, OH), 5.25 (m, 2H, 2×OH), 6.06 (m, 1H, NH), 6.52-6.55 (d, J=9 Hz, 2H, ArH), 7.03-7.06 (d, J=9 Hz, 2H, ArH), 7.94 (s, 1H, NH); Mass: m/z 353 (M⁺+1).

### Example 3E

### Preparation of 6-N-{[4-Fluorophenyl]aminocarbonylamino}-N-propyl-D-gluco-δ-lactam (Compound No. 29)

The title compound was prepared, and exhibited an m.p.: 138-139° C. The following spectral information was used to confirm product formation: IR(KBr): ν 1651 cm⁻¹; ¹HNMR (CDCl₃): δ 0.72-0.77 (t, J=7 Hz, 3H, CH₃), 1.35-1.50 (m, 2H, 2×CH₂), 2.84-2.93 (m, 1H, CH), 3.37-3.69 (m, 6H, 6×CH), 3.89-3.93 (d, J=7 Hz, 1H, CH), 6.95-7.01 (m, 2H, ArH), 7.10-7.15 (m, 2H, ArH); Mass: m/z 356 (M⁺+1).

### Example 3F

### Preparation of 6-N-{[Isopropyl]aminocarbonylamino}-N-propyl-D-gluco-δ-lactam (Compound No. 30)

The title compound was prepared, and exhibited an m.p.: 151° C. The following spectral information was used to confirm product formation: IR(CH₂Cl₂): ν 1638.8 cm⁻¹; ¹HNMR (D₂O): δ 0.83-0.87 (t, J=7 Hz, 3H, CH₃), 1.07-1.09 (m, 6H, CH₃), 1.45-1.60 (m, 2H, 2×CH), 2.92-3.01 (m, 1H, CH), 3.45 (m, 3H, 3×CH), 3.54-3.60 (m, 1H, CH), 3.65-3.75 (m, 3H, 3×CH), 3.96-4.00 (d, J=12 Hz, 1H, CH); Mass: m/z 304 (M⁺+1).

### Example 3G

### Preparation of 6-N-{[4-Trifluoromethylphenyl]aminocarbonylamino}-N-propyl-D-gluco-δ-lactam (Compound No. 31)

The title compound was prepared, and exhibited an m.p.: 189° C. The following spectral information was used to confirm product formation: IR(KBr): ν 1691, 1651, 1556 cm⁻¹; ¹HNMR (CD₃)₂δSO: δ 0.79-0.84 (t, J=7 Hz, 3H, CH₃), 1.44-1.53 (m, 2H, CH₂), 2.96-2.97 (m, 1H, CH), 3.30-3.44 (m, 3H, 3×CH), 3.54-3.61 (m, 3H, 3×CH), 3.72-3.77 (dd, J=3.6 Hz, 9 Hz, 1H, OH), 4.79-4.80 (d, J=4 Hz, 1H, OH), 5.32-5.33 (d, J=4 Hz, 1H, OH), 5.39-5.41 (m, 1H, OH), 6.37-6.38 (m, 1H, NH), 8.99 (s, 1H, NH), 7.57 (m, 4H, ArH); Mass: m/z 406 (M⁺+1).

### Example 4

### Preparation of 2,3,4-tri-O-acetyl-6-N-{[4-methoxyphenyl]aminocarbonylamino}-N-propyl-D-gluco-δ-lactam (Compound No. 32)

To a solution of 6-N-{[-methoxyphenyl]aminocarbonylamino}-N-propyl-D-gluco-δ-lactam (1 mmol), prepared as described in Example 3 in pyridine (5 ml) was added acetic anhydride (2 ml) at 0° C. The reaction mixture was allowed to come at room temperature and stirred for 2 hours. Pyridine was distilled off and the residue was diluted with water (20 ml) and extracted with ethyl acetate (2 times 25 ml). The organic layer was dried over anhydrous Na₂SO₄, solvent removed under vacuo and the compound obtained was purified by column chromatography using ethylacetate as an eluent. The product had an m.p.: 80° C.

The following spectral information was used to confirm product formation: R(KBr): ν 1754, 1666, 1555 cm⁻¹; ¹HNMR (CDCl₃): δ 0.84-0.88 (t, J=7 Hz, 3H, CH₃), 1.50-1.60 (m, 2H, CH₂), 2.06 (s, 3H, CH₃), 2.09 (s, 3H, CH₃), 2.14 (s, 3H, CH₃), 2.83-2.87 (m, 1H, CH), 3.50-3.66 (m, 2H, 2×CH), 3.78 (s, 3H, CH₃), 3.82-3.87 (m, 2H, 2×CH), 5.13 (m, 1H, CH), 5.24-5.28 (m, 1H, CH), 5.58-5.61 (d, J=9 Hz, 1H, CH), 6.83-6.86 (d, J=9 Hz, 2H, ArH), 7.24-7.27 (d, J=9 Hz, 2H, ArH); Mass: m/z 494 (M⁺+1).

The following compounds were prepared analogously, as will be understood by one of ordinary skill in the art:

### Example 4A

### Preparation of 2,3,4-Tri-O-acetyl-6-N-{[phenyl]aminocarbonylamino}-N-propyl-D-gluco-δ-lactam (Compound No. 33)

The title comopound was prepared, and exhibited an m.p.: 73° C. The following spectral information was used to confirm product formation: IR(KBr): ν 1752, 1660 cm⁻¹; ¹HNMR (CDCl₃): δ 0.82-0.89 (t, J=7 Hz, 3H, CH₃), 1.51-1.61 (m, 2H, CH₂), 2.08 (s, 3H, CH₃), 2.10 (s, 3H, CH₃), 2.13 (s, 3H, CH₃), 2.78-2.85 (m, 1H, CH), 3.47-3.55 (dd, J=8 Hz, 14 Hz, 1H, CH), 3.67-3.73 (dd, J=6 Hz, 14 Hz, 1H, CH), 3.84-3.91 (m, 2H, 2×CH), 5.14-5.15 (m, 1H, CH), 5.27-5.33 (m, 1H, CR), 5.64-5.68 (dd, J=3.6 Hz, 9 Hz, 1H, CH, 7.00-7.05 (m, 1H, NH), 7.20-7.39 (m, 5H, ArH); Mass: m/z 464 (M⁺+1).

### Example 4B

### Preparation of 2,3,4-Tri-O-acetyl-6-N-{[4-fluorophenyl]aminocarbonylamino}-N-propyl-D-gluco-δ-lactam (Compound No. 34)

The title compound was prepared, and exhibited an m.p.: 79-81° C. The following spectral information was used to confirm product formation: IR(KBr): ν 1748, 1651, 1557 cm⁻¹; ¹HNMR (CDCl₃): δ 0.81-0.86 (t, J=7 Hz, 3H, CH₃), 1.48-1.60 (m, 2H, CH₂), 2.09 (s, 3H, CH₃), 2.10 (s, 3H, CH₃), 2.13 (s, 3H, CH₃), 2.77-2.87 (m, 1H, CH), 3.47-3.57 (m, 1H, CH), 3.68-3.74 (m, 1H, CH), 3.80-3.90 (m, 2H, 2×CH), 5.13-5.16 (t, J=3 Hz, 1H, CH), 5.28-5.32 (dd, J=3 Hz, 9 Hz, 1H, CH) 5.63-5.66 (d, J=3 Hz, 9 Hz, 1H, CH), 6.93-6.99 (t, J=9 Hz, 2H, ArH), 7.31-7.36 (m, 2H, ArH), 7.71 (s, 1H, NH); Mass: m/z 482 (M⁺+1).

### Example 5

### Preparation of 2,3,4-tri-O-trimethylsilyl-6-N-{[phenyl]aminocarbonylamino}-N-propyl-D-gluco-δ-lactam (Compound No. 35)

N-Propyl-6-N[{[phenyl}aminocarbonyl amino]-D-gluco-δ-lactam (1 mmol), obtained as described in Example 3, was dissolved in pyridine (10 ml) and the reaction mixture was cooled to 0° C. To this was added trimethyl silyl chloride (TMSCl) (6 mmol) and the reaction mixture was allowed to come to room temperature. Stirring was done at this temperature for 3 hours. The solvent was removed under reduced pressure. Residue was dissolved in water (30 ml) and compound extracted with ethylacetate (2 time 25 ml). Organic layer was dried, solvent removed under reduced pressure. The product was purified by column chromatography using hexane-ethylacetate (9:1) as eluent to get the product as an oily compound.

The following spectral information was used to confirm product formation: IR(CH₂Cl₂): ν 1643.5 cm⁻¹; ¹HNMR (CDCl₃): δ 0.37-0.62 (m, 27H, 9×CH₃), 1.14-1.25 (m, 3H, CH₃), 1.87-1.89 (m, 2H, 2×CH), 3.25 (m, 1H, CH), 3.80-3.94 (m, 3H, 3×CH), 4.06-4.15 (m, 3H, 3×CH), 4.33-4.35 (d, J=6 Hz, 1H, CH), 5.65-5.69 (m, 1H, NH), 5.83-5.87 (m, 1H, NH), 7.46-7.69 (m, 5H, ArH); Mass: m/z 554 (M⁺+1).

### Example 6

### Preparation of 2,3,4-tri-O-benzyl-6-N-{[4-chlorophenyl]aminocarbonylamino}-1,5-dideoxy-1,5-imino glucitol (Compound No. 36)

The ester obtained in Example 2 (1 mmol) was dissolved in anhydrous tetrahydrofuran (THF) (15 ml). To this was added lithium aluminium hydride (LAH) (2 mmol). The reaction mixture was heated to reflux temperature and stirred for 2 hours at this temperature. Now the reaction mixture was stirred for another 1 hour. The compound was extracted with ethyl acetate (2 times 25 ml), dried over Na₂SO₄ and solvent was removed under reduced pressure. The crude material was purified by column chromatography using ethylacetate-hexane (3:7) as an eluent to give the title compound in semisolid state.

The following spectral information was used to confirm product formation: IR(CH₂Cl₂): ν 1652 cm⁻¹; ¹HNMR (CDCl₃):δ 0.79-0.83 (t, J=7 Hz, 3H, CH₃), 1.42-1.43 (m, 2H, CH₂), 2.27 (m, 1H, NCH), 2.43-2.47 (m, 2H, 2×CH), 2.63 (m, 1H, CH), 3.09-3.13 (m, 1H, CH), 3.30-3.34 (m, 1H, CH), 3.43-3.53 (m, 3H, 3×CH), 3.77-3.81 (m, 1H, CH), 4.61-4.65 (d, J=12 Hz, 1H, PhCH), 4.69-4.83 (m, 3H, 3×PhCH), 4.85-4.89 (d, J=12 Hz, 1H, PhCH), 4.93-4.97 (d, J=12 Hz, 1H, NH), 5.30 (brs, 1H, NH), 6.56 (brs, 1H, NH), 7.22-7.33 (m, 19H, ArH); Mass: m/z 628 (M⁺+1).

The following compounds were prepared analogously, as will be understood by one of ordinary skill in the art:

### Example 5A

### Preparation of 2,3,4-Tri-O-benzyl-6-N-{[2,4-difluorophenyl]aminocarbonylamino}-1,5-dideoxy-1,5-imino glucitol (Compound No. 37)

The title compound was formed, and the following spectral information was used to confirm product formation: IR(CH₂Cl₂): ν 1644 cm⁻¹; ¹HNMR (CDCl₃): δ 0.80-0.85 (t, J=7 Hz, 3H, CH₃), 1.41-1.48 (m, 2H, CH₂), 2.28 (m, 1H, CH), 2.48 (m, 2H, 2×CH), 2.66 (m, 1H, CH), 3.11-3.14 (m, 1H, CH), 3.32-3.57 (m, 4H, 4×CH), 3.78-3.84 (m, 1H, CH), 4.62-4.71 (m, 3H, 3×PhCH), 4.81-4.89 (m, 2H, 2×PhCH), 4.94-4.98 (d, J=12 Hz, 1H, PhCH), 5.17 (brs, 1H, NH), 6.50 (brs, 1H, NH), 6.78-6.84 (t, J=9 Hz, 2H, ArH), 7.17-7.33 (m, 15H, ArH), 7.90-7.95 (m, 1H, ArH); Mass: m/z 630 (M⁺+1).

### Example 5B

### Preparation of 2,3,4-Tri-O-benzyl-6-N-{[isopropyl]aminocarbonylamino}-1,5-dideoxy-1,5-imino-N-propyl glucitol (Compound No. 38)

The title compound was prepared, and the following spectral information was used to confirm product formation: IR(CH₂Cl₂): ν 1701, 1652 cm⁻¹; ¹HNMR (CDCl₃): δ 0.82-0.87 (t, J=7 Hz, 3H, CH₃), 1.43-1.46 (m, 2H, CH₂), 2.25 (m, 1H, NCH), 2.43 (m, 2H, 2×CH), 2.62 (m, 1H, CH), 3.10 (m, 1H, CH), 3.31-3.56 (m, 4H, 4×CH), 3.79-3.89 (m, 4H, OCH₃+CH), 4.58-4.89 (m, 5H, 5×PhCH), 4.96-5.00 (d, J=12 Hz, 1H, PhCH), 6.71-6.74 (d, J=9 Hz, 2H, ArH), 6.88-6.91 (m, 1H, ArH), 7.14-7.42 (m, 15H, ArH), 8.14 (m, 1H, NH); Mass: m/z 658 (M⁺+1).

### Example 5C

### Preparation of 2,3,4-Tri-O-benzyl-6-N-{[4-chlorophenyl]aminothiocarbonylamino}-1,5-dideoxy-1,5-imino-N-propyl glucitol (Compound No. 39)

The title compound was prepared, and the following spectral information was used to confirm product formation: IR(CH₂Cl₂): ν 1626, 1519, 1492 cm⁻¹; ¹HNMR (CDCl₃): δ 0.57-0.61 (t, J=7 Hz, 3H, CH₃), 1.14-1.66 (m, 1H, CH₂), 1.25 (m, 1H, CH), 2.00-2.04 (m, 2H, 2×CH), 2.35-2.39 (m, 2H, CH), 2.92-2.96 (m, 1H, CH), 3.16 (m, 1H, CH), 3.21 (m, 1H, CH), 3.44-3.49 (m, 2H, CH), 4.35 (m, 1H, CH), 4.62-4.66 (d, J=12 Hz, 1H, PhCH), 4.69-4.83 (m, 3H, 3×PhCH), 4.86-4.90 (d, J=12 Hz, 1H, PhCH), 6.64-6.66 (m, 1H, NH), 7.06-7.09 (d, J=9 Hz, 2H, ArH), 7.25-7.37 (m, 17H, ArH), 7.76 (s, 1H, NH); Mass: m/z 644 (M⁺+1).

### Example 5D

### Preparation of 2,3,4-Tri-O-benzyl-6-N-{[4-fluorophenyl]aminocarbonylamino}-1,5-dideoxy-1,5-imino-N-propyl glucitol (Compound No. 40)

The title compound was prepared, and the following spectral information was used to confirm product formation: IR(CH₂Cl₂): ν 1650 cm⁻¹; ¹HNMR (CDCl₃): δ 0.81-0.86 (t, J=7 Hz, 3H, CH₃), 1.41-1.44 (m, 2H, CH₂), 2.63-2.73 (m, 2H, 2×CH), 2.90 (m, 2H, 2×CH), 3.29 (m, 1H, CH), 3.41-3.46 (m, 1H, CH), 3.61 (m, 2H, 2×CH), 3.78-3.82 (m, 2H, 2×CH), 4.60-4.64 (d, J=12 Hz, 1H, PhCH), 4.67-4.86 (m, 5H, 5×PhCH), 6.93-7.15 (m, 2H, ArH), 7.25-7.39 (m 17H, ArH); Mass: m/z 612 (M⁺+1).

### Example 7

### Preparation of N-allyl-6-O-(4-chlorophenyl carbamate)-2,3,4-tri-O-benzyl-D-gluco-δ-lactam (Compound No. 41)

N-Allyl-2,3,4-tri-O-benzyl-D-gluco-δ-lactam (0.487 gm) was dissolved in acetonitrile (10 ml). To this was added p-chlorophenyl isocyanate (0.184 gm) and triethylamine (0.203 gm) and stirred the reaction mixture for 1 hour at room temperature. Acetonitrile was removed under vacuo. The residue left was diluted with water (50 ml) and the product was extracted with ethyl acetate (2 times, 25 ml). Organic layer was dried over anhydrous Na₂SO₄ and the solvent was evaporated under reduced pressure. The compound was purified using column chromatography with ethyl acetate-hexane (2:8) as the eluent mixture to give the title compound in semisolid state.

The following spectral information was used to confirm product formation: IR(CH₂Cl₂): ν 1734, 1659 cm⁻¹; ¹HNMR (CDCl₃): δ 3.60 (m, 1H, CH), 3.68-3.71 (m, 1H, CH), 3.70-3.78 (m, 1H, CH), 3.92-3.93 (m, 1H, CH), 4.07 (d, 1H, CH), 4.26-4.28 (m, 2H, 2×CH), 4.56-4.78 (m, 5H, 4×PhCH+CH) 4.82-4.86 (d, J=12 Hz, 1H, PhCH), 5.16-5.31 (m, 3H, 2×CH+PhCH), 5.77-5.82 (m, 1H, CH), 6.56 (s, 1H, NH), 7.17-7.46 (m, 19H, ArH); Mass: m/z 641.5 (M⁺+1).

The following compound was prepared analogously, as will be recognized by one of ordinary skill in the art:

### Example 7A

### Preparation of N-Allyl-2,3,4-tri-O-benzyl-6-(2-naphthylthiocarbamate)-D-gluco-δ-lactam (Compound No. 42)

The title compound was prepared by using 2-naphthyl isothiocyanate instead of p-chlorophenyl isocyanate in the above Example 7 to give the title compound in semisolid state. The following spectral information was used to confirm product formation: ¹HNMR (CDCl₃): δ 3.26 (m, 1H, CH), 3.41 (m, 1H, CH), 3.49 (m, 2H, 2×CH), 3.65 (m, 1H, CH), 3.89 (m, 1H, CH), 4.10 (m, 2H, 2×CH), 4.23 (m, 2H, CH₂), 4.34-4.59 (m, 3H, 3×CH), 4.87 (m, 2H, 2×CH), 4.97-5.01 (m, 2H, 2×CH), 5.52 (m, 1H, CH), 7.00-7.87 (m, 22H, ArH); Mass: m/z 672 (M⁺+1).

### Example 8

### Preparation of N-Propyl-6-(phenyl carbamate -N-propyl-D-gluco-δ-lactam (Compound No. 43)

The ester obtained in Example 7, was debenzylated by the same procedure as described in Example 3 to give the title compound. The title compound had m.p.: 134-135° C. The following spectral information was used to confirm product formation: IR(CH₂Cl₂): ν 3308, 1747, 1641 cm⁻¹; ¹HNMR (CDCl₃): δ 0.87-0.91 (t, J=7 Hz, 3H, CH₃), 1.52-1.62 (m, 2H, CH₂), 3.04-3.07 (m, 1H, NCH), 3.45-3.47 (m, 1H, NCH), 3.55-3.62 (m, 1H, CH), 3.72-3.75 (m, 1H, CH), 3.87-3.96 (m, 2H, 2×CH), 3.99 (brs, 1H, OH), 4.17 (s, 2H, 2×CH), 4.37 (brs, 1H, OH), 5.02 (brs, 1H, OH) 6.99-7.04 (t, J=7 Hz, 1H, ArH), 7.19-7.29 (m, 2H, ArH), 7.44 (m, 2H, ArH), 8.74 (s, 1H, NH); Mass: m/z 339 (M⁺+1).

### Example 9

### Preparation of N-allyl-2,3,4-tri-O-benzyl-6-{p-chlorophenylcarbamate}-1,5-dideoxy-1,5-imino glucitol (Compound No. 44)

The ester obtained in Example 7 was reduced by following the same procedure as described in Example 6, to yield the title compound, which had an m.p.: 106-107° C. The following spectral information was used to confirm product formation: IR(CH₂Cl₂): ν 1733 cm⁻¹; ¹HNMR (CDCl₃): δ 2.17-2.25 (t, J=15 Hz, 1H, NCH), 2.43 (m, 1H, NCH), 3.13-3.23 (m, 2H, 2×CH), 3.37-3.43 (m, 1H, CH), 3.51-3.61 (m, 3H, 3×CH), 4.39-4.49 (Abq, 2H, NCH₂), 4.60-4.64 (d, J=12 Hz, 1H, PhCH), 4.67-4.68 (m, 2H, 2×PhCH), 4.83-4.92 (m, 2H, 2×PhCH), 4.98-5.02 (d, J=12 Hz, 1H, PhCH), 5.20-5.24 (m, 2H, 2×CH), 5.82-5.84 (m, 1H, CH), 6.70 (s, 1H, NH), 7.21-7.45 (m, 19H, ArH); Mass: m/z 627 (M⁺+1).

### Example 10

### Preparation of N-allyl-6-O-(2-thiophenyl)-2,3,4-tri-O-benzyl-D-gluco-δ-lactam (Compound No. 45)

Dissolved N-allyl-2,3,4-tri-O-benzyl-D-gluco-δ-lactam (0.487 gm) in dichloromethane (DCM, 10 ml). To this was added triethylamine (TEA) (0.202 gm) and thiophene-2-carbonyl chloride (0.220 gm) and stirred the reaction mixture for 3 hours at room temperature. The reaction mixture was diluted with water (25 ml) and the product was extracted with dichloromethane (2 times, 25 ml). The organic layer was dried over anhydrous Na₂SO₄, and solvent was distilled off under reduced pressure. The product obtained was purified by coulmn chromatography using ethylacetate-hexane (1:9) as eluent to give the title compound in semisolid state.

The following spectral information was used to confirm product formation: IR(CH₂Cl₂): ν 1714, 1672 cm⁻¹; ¹HNMR (CDCl₃): δ 3.64-3.78 (m, 2H, 2×CH), 3.83-3.92 (m, 2H, 2×CH), 4.10-4.13 (d, J=9 Hz, 1H, CH), 4.29-4.42 (m, 2H, 2×CH), 4.50-4.75 (m, 6H, 4×PhCH+2×CH), 4.79-4.83 (d, J=12 Hz, 1H, PhCH), 5.16-5.20 (m, 2H, PhCH+CH), 5.72-5.80 (m, 1H, CH), 7.09-7.74 (m, 18H, ArH); Mass: m/z 598 (M⁺+1).

### Example 11

### Preparation of 2,3,4-tri-O-benzyl-N-propyl-6-{[2-thiophene]carboxamido}-D-gluco-δ-lactam (Compound No. 46)

6-Amino-N-propyl-2,3,4-tri-O-benzyl-D-gluco-δ-lactam (1 mmol). Prepared as described in Step 3, Example 2 was dissolved in dichloromethane (DCM) (10 ml). To this was added triethylamine (TEA) (2 mmol) and thiophene-2-carbonyl chloride (1.5 mmol) and the reaction mixture was stirred for 1 hour at room temperature. This was then diluted with water (25 ml) and the compound was extracted with dichloromethane (2 times, 25 ml). The organic layer was dried over anhydrous Na₂SO₄ and the solvent was distilled off under reduced pressure. The crude material was purified by column chromatography with ethylacetate-hexane (4:6) as an eluent mixture to give the title compound in semisolid state.

The following spectral information was used to confirm product formation: IR(CH₂Cl₂): ν 1718, 1649 cm⁻¹; ¹HNMR (CDCl₃): δ 0.88-0.93 (t, J=7 Hz, 3H, CH₃), 1.57-1.65 (m, 2H, CH₂), 3.08-3.12 (m, 1H, NCH), 3.34-3.38 (m, 1H, NCH), 3.68-3.92 (m, 5H, CH₂+3×CH), 4.07-4.09 (d, J=6 Hz, 1H, CH), 4.48-4.68 (m, 3H, 3×PhCH), 4.71-4.75 (m, 2H, 2×PhCH), 5.11-5.15 (d, J=12 Hz, 1H, PhCH), 6.21 (brs, 1H, NH), 7.00-7.46 (m, 18H, ArH); Mass: m/z 599 (M⁺+1).

The following compounds were prepared analogously, as will be understood by one of ordinary skill in the art:

### Example 11A

### Preparation of 2,3,4-Tri-O-benzyl-6-N-{[2,4-difluorophenyl]carboxamido}-N-propyl-D-gluco-δ-lactam (Compound No. 47)

The title compound was prepared by using 2,4-difluorophenyl carbonyl chloride instead of thiophene-2-carbonyl chloride in Example 11 to give the title compound in semisolid state. The following spectral information was used to confirm product formation: IR(CH₂Cl₂): ν 1671, 1659 cm⁻¹; ¹HNMR (CDCl₃): δ 0.89-0.94 (t, J=7 Hz, 3H, CH₃), 1.63-1.65 (m, 2H, CH₂), 3.09 (m, 1H, NCH), 3.37 (m, 1H, NCH), 3.68-3.70 (m, 1H, CH), 3.81 (m, 2H, 2×CH), 3.89-3.92 (m, 2H, 2×CH), 4.06-4.09 (d, J=9 Hz, 1H, CH), 4.58-4.55 (d, J=9 Hz, 1H, CH), 4.58-4.55 (d, J=12 Hz, 1H, PhCH), 4.69-4.74 (m, 4H, 4×PhCH), 5.13-5.17 (d, J=12 Hz, 1H, PhCH), 7.01-7.45 (m, 17H, ArH), 8.06-8.08 (m, 1H, ArH); Mass: m/z 629 (M⁺+1).

### Example 11B

### Preparation of 2,3,4-Tri-O-benzyl-6-(adamantanecarboxamido}-N-propyl-D-gluco-δ-lactam (Compound No. 48)

The title compound was synthesized by using adamantane 1-carbonyl chloride in place of thiophene-2-carbonyl chloride in Example 11 to give the title compound in semisolid state. The following spectral information was used to confirm product formation: IR(CH₂Cl₂): ν 1658, 1519 cm⁻¹; ¹HNMR (CDCl₃): δ 0.88-0.93 (t, J=7 Hz, 3H, CH₃), 1.56-1.71 (m, 14H, 14×CH), 1.94 (m, 2H, 2×CH), 3.07-3.09 (m, 2H, 2×CH), 3.58-3.65 (m, 1H, CH), 3.71 (m, 3H, 3×CH), 3.91 (m, 1H, CH), 4.05-4.07 (d, J=6 Hz, 1H, CH), 4.47-4.51 (d, J=2 Hz, 1H, PhCH), 4.53 (m, 2H, 2×PhH), 4.64-4.68 (d, J=12 Hz, 1H, PhCH), 4.74-4.78 (d, J=12 Hz, 1H, PhCH), 5.13-5.17 (d, J=12 Hz, 1H, PhCH), 5.90 (m, 1H, NH), 7.20-7.44 (m, 15H, ArH); Mass: m/z 651 (M⁺+1).

### Example 12

### Preparation of 6-N-{[2,4-difluorophenyl]aminocarbonylamino}-N-propyl-D-gluco-δ-lactam (Compound No. 49)

The ester obtained in Example 11 was debenzylated by the same procedure as described in Example 3 to give the title compound in a semisolid state. The following spectral information was used to confirm product formation: IR(CH₂Cl₂): ν 3378, 1647, 1620 cm⁻¹; ¹HNMR (CDCl₃): δ 0.86-0.93 (m, 3H, CH₃), 1.54-1.60 (m, 2H, CH₂), 3.11-3.12 (m, 1H, NCH), 3.49-3.55 (m, 2H, NCH+CH), 3.65-3.93 (m, 5H, 5×CH), 4.08 (brs, 3H, 3×OH), 6.80-6.87 (m, 1H, ArH1), 6.94-6.99 (m, 1H, ArH), 7.12 (brs, 1H, NH), 7.97-8.05 (m, 1H, ArH); Mass: m/z 359 (M⁺+1).

The following compound was prepared analogously, as will be clear to one of ordinary skill in the art:

### Example 12A

### Preparation of 6-(Adamantane carboxamido)-N-propyl-D-gluco-δ-lactam (Compound No. 50)

The title compound was prepared, and the following spectral information was used to confirm produt formation: IR(CH₂Cl₂): ν 3422, 1637 cm⁻¹; ¹HNMR (CDCl₃): δ 0.90-0.95 (t, J=7 Hz, 3H, CH₃), 1.61-1.82 (m, 14H, 14×CH), 2.05 (m, 3H, 3×CH), 3.24 (m, 1H, CH), 3.29 (m, 1H, CH), 3.37 (m, 1H, CH), 3.53 (m, 1H, CH), 3.55-3.58 (m, 1H, CH), 3.64-3.70 (m, 1H, CH), 3.83-3.86 (m, 1H, CH), 3.95-4.00 (m, 1H, CH), 6.00-4.04 (m, 1H, NH); Mass: m/z 381 (M⁺+1).

### Example 13

### Preparation of 2,3,4-tri-O-benzyl-6-(trifluoromethylacetamido-N-propyl-D-gluco-δ-lactam (Compound No. 51)

6-Amino-2,3,4-tri-O-benzyl-N-propyl-D-gluco-δ-lactam (0.488 gm) obtained in step 3, Example 2 was dissolved in dichloromethane (DCM) (10 ml). The reaction mixture was cooled to 0° C. and added triethylamine (TEA) (0.404 gm) and trifluoroacetic anhydride (0.525 gm) to it. The reaction was allowed to come to room temperature ad stirred for 1 hour. The reaction mixture was then diluted with water (25 ml) and the product was extracted with dichloromethane (DCM) (2 times, 25 ml). The organic layer was dried over anhydrous Na₂SO₄ and the solvent was removed under vacuo. The compound obtained was purified by column chromatography using ethylacetate-hexane (1:9) as the eluent mixture to yield the title compound in semisolid state.

The following spectral information was used to confirm product formation: ¹HNMR (CDCl₃): δ 0.88-0.92 (t, J=7 Hz, 3H, CH₃), 1.23-1.30 (m, 2H, CH₂), 2.99 (m, 1H, NCH), 3.39-3.41 (m, 2H, 2×CH), 3.56 (s, 1H, CH), 3.63-3.70 (m, 2H, CH), 3.91 (m, 1H, CH), 4.01-4.09 (m, 2H, 2×CH), 4.44-4.48 (d, J=12 Hz, 1H, PhCH), 4.54-4.65 (m, 3H, 3×PhCH), 4.72-4.76 (d, J=12 Hz, 1H, PhCH), 5.09-5.13 (d, J=12 Hz, 1H, PhCH), 6.95 (brs, 1H, NH), 7.19-7.89 (m, 15H, ArH); Mass: m/z 585 (M⁺+1).

### Example 14

### Preparation of 2,3,4-tri-O-benzyl-6-N-{2-[chloro]-acetyl}-N-propyl-D-gluco-δ-lactam (Compound No. 52)

6-Amino-2,3,4-tri-O-benzyl-N-propyl-D-gluco-δ-lactam (0.488 gm), obtained as described in Step 3, Example 2 was dissolved in dichloromethane (DCM) (15 ml). The reaction mixture was cooled to 0° C. and added triethylamine (0.203 gm) to it. Chloroacetylchloride (1.2 mmol) was added dropwise to the reaction mixture and the reaction mixture was allowed to come to room temperature at which it was stirred for 1 hour. The contents of the reaction mixture were diluted with cold water (25 ml) and the compound was extracted with dichloromethane (2 times, 50 ml). The organic layer was dried over Na₂SO₄ and solvent removed under vacuo. The product was purified by column chromatography with ethylacetate-hexane (4:6) as eluent to yield the title compound in a semisolid state.

The following spectral information was used to confirm product formation: IR(CH₂Cl₂): ν 1682, 1652 cm⁻¹; ¹HNMR (CDCl₃): δ 0.90-0.95 (t, J=7 Hz, 3H, CH₃), 1.60-1.69 (m, 2H, CH₂), 3.04 (m, 1H, NCH), 3.32 (m, 1H, NCH), 3.62-3.69 (m, 4H, 4×CH), 3.92-3.93 (m, 1H, CH), 3.96 (s, 2H, CH₂), 4.07-4.09 (d, 1H, CH), 4.48-4.62 (m, 3H, 3×PhCH), 4.69-4.78 (m, 2H, 2×PhCH), 5.13-5.17 (d, J=12 Hz, 1H, PhCH), 6.76 (brm, 1H, NH), 7.25-7.47 (m, 15H, ArH); Mass: m/z 565 (M⁺+1).

### Example 15

### Preparation of 2,3,4-tri-O-benzyl-N-propyl-6-{2-[triazolyl]-acetyl}-D-gluco-δ-lactam (Compound No. 53)

2,3,4-Tri-O-benzyl-6-N-{2-[chloro]-acetyl}-N-propyl-D-gluco-δ-lactam (1 mmol), prepared as described in Example 14 was dissolved in DMF. To this was added triazole (2 mmol), potassium carbonate (2 mmol) and tetrabutyl ammonium iodide (catalytic amount) and stirred the reaction mixture at room temperature for 12 hours. DMF was removed under vacuo and the residue was diluted with water (25 ml). The compound was extracted with ethylacetate (2 times, 25 ml), organic layer dried over Na₂SO₄ and solvent removed under reduced pressure. The product was purified by column chromatography using chloroform-methanol (9.5:0.5) as the eluent to yield the title compound in semisolid state.

The following spectral information was used to confirm product formation: IR(CH₂Cl₂): ν 1662 cm⁻¹; ¹HNMR (CDCl₃): δ 0.86-0.91 (t, J=7 Hz 3H, CH₃), 1.55-1.58 (m, 2H, CH₂), 2.91-2.93 (m, 1H, NCH₂), 3.24-3.27 (m, 1H, NCH₂), 3.52 (m, 1H, CH), 3.65-3.71 (m, 3H, 3×CH), 3.85-3.87 (m, 1H, CH), 3.99-4.01 (d, 1H, CH), 4.44-4.55 (m, 3H, CH₂+PhCH), 4.59-4.72 (m, 4H, PhCH), 4.59-4.72 (m, 4H, PhCH), 5.14-5.19 (d, J=15 Hz, 1H, PhCH), 6.68 (brm, 1H, NH), 7.14-7.44 (m, 15H, ArH) 7.81 (s, 1H, TrH), 8.07 (s, 1H, TrH); Mass: m/z 598 (M⁺+1).

The following compounds were synthesized analogously by using different heterocycles/compounds.

### Example 15A

### Preparation of 2,3,4-Tri-O-benzyl-6-N-{2-[4-(pyrimidyl)piperazinyl]acetyl}-N-proyl-D-gluco-δ-lactam (Compound No. 54)

The title compound was prepared, and the following spectral information was used to confirm product formation: IR(CH₂Cl₂): ν 1645, 1586 cm⁻; ¹HNMR (CDCl₃): δ 0.90-0.95 (t, J=7 Hz, 3H, CH₃), 1.65 (m, 2H, CH₂), 2.36-2.48 (m, 4H, 4×CH), 2.95-2.97 (m, 2H, 2×CH), 3.03-3.10 (m, 1H, CH), 3.21-3.25 (m, 1H, CH), 3.64-3.81 (m, 8H, 4×CH₂), 3.89-3.93 (m, 1H, CH), 4.50-4.54 (d, J=12 Hz, 1H, PhCH), 4.55-4.63 (m, 2H, 2×PhCH), 4.70-4.76 (m, 2H, 2×CH), 5.15-5.19 (d, J=12 Hz, 1H, PhCH), 6.51-6.54 (t, J=7 Hz, 1H, NH), 7.18-7.46 (m, 16H, ArH), 8.31-8.32 (d, J=6 Hz, 2H, ArH); Mass: m/z 693 (M⁺+1).

### Example 15B

### Preparation of 2,3,4-Tri-O-benzyl-6-N-{2-[4-fluoroanilino]acetyl}-N-propyl-D-gluco-δ-lactam (Compound No. 55)

The title compound was prepared, and the following spectral information was used to confirm product formation: IR(CH₂Cl₂): ν 1656 cm⁻¹; ¹HNMR (CDCl₃): δ 6 0.86-0.91 (t, J=7 Hz, 3H, CH₃), 1.60 (m, 2H, CH₂), 2.92-3.01 (m, 1H, CH), 3.23-3.34 (m, 1H, CH), 3.48-3.50 (t, J=3 Hz, 1H, CH), 3.48-3.50 (t, J=3 Hz, 1H, CH), 3.64-3.72 (m, 5H, 5×PhCH), 5.07-5.11 (d, J=12 Hz, 1H, PhCH), 5.07-5.11 (d, J=12 Hz, 1H, PhCH), 6.40-6.45 (m, 2H, NH), 6.87-6.90 (m, 4H, ArH), 7.16-7.43 (m, 15H, ArH); Mass: m/z 640 (M⁺+1).

### Example 15C

### Preparation of 2,3,4-Tri-O-benzyl-6-{2-[2,6-diketopiperidino]acetyl}-N-propyl-D-gluco-δ-lactam (Compound No. 56)

The title compound was prepared, and the following spectral information was used to confirm product formation: IR(CH₂Cl₂): ν 1731, 1678 cm⁻¹; ¹HNMR (CDCl₃): δ 0.88-0.93 (t, J=7 Hz, 3H, CH₃), 1.50-1.58 (m, 2H, CH₂), 1.93-2.02 (p, J=6 Hz, 2H, CH₂), 2.65-2.70 (t, J=6 Hz, 4H, 2×CH₂), 2.98-3.07 (m, 1H, CH), 3.11-3.17 (m, 1H, CH), 3.62-3.68 (m, 4H, 4×CH), 3.86-3.89 (m, 1H, CH), 4.04-4.06 (d, J=7 Hz, 1H, CH), 4.57 (Abq, J=3.6 Hz, 18 Hz, 2H, CH₂), 4.69-4.72 (d, J=7 Hz, 1H, PhCH), 4.57 (s, 2H, 2×PhCH), 4.69-4.72 (d, J=7 Hz, 1H, PhCH), 4.73-4.75 (d, J=7 Hz, 1H, PhCH), 5.12-5.16 (d, J=12 Hz, 1H, PhCH), 5.68 (m, 1H, NH), 7.14-7.32 (m, 13H, ArH), 7.43-7.45 (d, J=6 Hz, 2H, ArH); Mass: m/z 642 (M⁺+1).

### Example 15D

### Preparation of 2,3,4-Tri-O-benzyl-6-N-{2-[4-chlorophenyl-3-(2H,4H)-1,2,4-triazol-3-onyl]acetyl}-N-propvl-D-gluco-δ-lactam (Compound No. 57)

The title compound was prepared, and the following spectral information was used to confirm product formation: ¹HNMR (CDCl₃): δ 0.85-0.90 (t, J=7 Hz, 3H, CH₃), 1.54-1.56 (m, 2H, CH₂), 2.98 (m, 1H, NCH), 3.26 (m, 1H, NCH), 3.63-3.69 (m, 5H, 5×CH), 4.03-4.05 (d, J=7 Hz, 1H, CH), 4.43-4.72 (m,7H, 5×PhCH+CH₂), 5.08-5.12 (d, J=12 Hz, 1H, PhCH), 6.22 (brs, 1H, NH), 7.18-7.42 (m, 19H, ArH), 7.49 (s, 1H, ArH); Mass: m/z 723 (M⁺+1).

### Example 15E

### Preparation of 2,3,4-Tri-O-benzyl-6-N-{2-[4-(4-fluorophenyl)-piperazin-1-yl]acetyl}-N-propyl-D-gluco-δ-lactam (Compound No. 58)

The title compound was prepared, and the following spectral information was used to confirm product formation: IR(CH₂Cl₂): ν 1659 cm⁻¹; ¹HNMR (CDCl₃): δ 0.90-0.94 (t, J=7 Hz, 3H, CH₃), 1.63-1.66 (m, 2H, CH₂), 2.48-2.54 (m, 4H, 4×CH), 2.88-2.92 (m, 4H, 4×CH), 2.98-3.00 (d, J=6 Hz, 2H, 2×CH), 3.03-3.08 (m, 1H, CH), 3.52 (m, 1H, CH), 3.59-3.61 (m, 1H, CH), 3.70-3.76 (m, 3H, CH), 3.89-3.92 (m, 2H, 2×CH), 4.04-4.06 (d, J=7 Hz, 1H, CH), 4.50-4.63 (m, 3H, 3×PhCH), 4.69-4.74 (dd, J=4 Hz, 12 Hz, 2H, 2×PhCH), 5.14-5.18 (d, J=12 Hz, 1H, PhCH), 6.76-6.99 (m, 2H, ArH), 7.22-7.33 (m, 13H, ArH), 7.42-7.44 (d, J=6 Hz, 2H, ArH); Mass: m/z 709 (M⁺+1).

### Example 15F

### Preparation of 2,3,4-Tri-O-benzyl-6-N-{2-[N-(methyl)piperazin-1-yl]acetyl}-N-propyl-D-gluco-δ-lactam (Compound No. 59)

The title compound was prepared, and the following spectral data was used to confirm product formation: IR(CH₂Cl₂): ν 1667 cm⁻¹; ¹HNMR (CDCl₃): δ 0.89-0.94 (t, J=7 Hz, 3H, CH₃), 1.62-1.64 (m, 2H, CH₂), 2.23-2.42 (m, 11H, 4×CH₂+CH₃), 2.93-2.95 (Abq, 2H, CH₂), 3.04 (m, 1H, CH), 3.22-3.24 (m, 1H, CH), 3.33-3.39 (m, 1H, CH), 3.59-3.60 (t, J=3 Hz, 1H, CH), 3.70-3.74 (m, 3H, 3×CH), 3.90-3.91 (m, 1H, CH), 4.03-4.05 (d, J=12 Hz, 1H, CH), 4.58-4.62 (d, J=12 Hz, 1H, CH), 4.71-4.76 (m, 3H, 3×CH), 5.14-5.18 (d, J=12 Hz, 1H, CH), 7.16-7.45 (m, 15H, ArH); Mass: m/z 629 (M⁺+1).

### Example 15G

### Preparation of 2,3,4-Tri-O-benzyl-6-N-{2-[4-(4-(chlorophenyl)-piperazin-1-yl)-phenyl)-3(2H,4M-1,2,4-triazol-3-onyl]acetyl}-H-propyl-D-gluco-δ-lactam (Compound No. 60)

The title compound was prepared, and the following spectral information was used to confirm product formation: IR(CH₂Cl₂): ν 1716, 1657 cm⁻¹; ¹HNMR (CDCl₃): δ 0.83-0.88 (t, J=7 Hz, 3H, CH₃), 1.53-1.48 (m, 2H, CH₂), 2.07 (m, 1H, CH), 3.05 (m, 1H, CH), 3.31-3.32 (m, 8H, 4×CH₂), 3.65-3.71 (m, 4H, CH₂+2×CH), 3.86 (m, 1H, CH), 4.03-4.06 (d, J=7 Hz, 1H, CH), 4.46-4.73 (m, 5H, 5×PhCH), 5.07-5.11 (d, J=12 Hz, 1H, PhCH), 6.20 (m, 1H, NH), 6.88-6.90 (d, J=9 Hz, 2H, ArH), 6.97-7.00 (d, J=9 Hz, 2H, ArH), 7.20-7.47 (m, 20H, ArH); Mass: m/z 884 (M⁺+1).

### Example 15H

### Preparation of 2,3,4-Tri-O-benzyl-6-N-{2-[2,3,4,6-tetra-O-benzyl-1,5-dideoxy-1,5-imino-glucitolyl]acetyl}-N-propyl-D-gluco-δ-lactam (Compound No. 61)

The title compound was prepared, and the following spectral information was used to confirm product formation: IR(CH₂Cl₂): ν 1670 cm⁻¹; ¹HNMR (CDCl₃): δ 0.84-0.89 (t, J=7 Hz, 3H, CH₃), 1.52-1.59 (m, 2H, CH₂), 2.04-2.07 (m, 1H, CH), 2.42 (s, 1H, CH), 2.52 (s, 1H, CH), 2.87-2.96 (m, 3H, 3×CH), 3.05-3.10 (m, 1H, CH), 3.53 (m, 2H, 2×CH), 3.56-3.67 (m, 6H), 3.68-3.72 (m, 1H, CH), 3.83 (m, 1H, CH), 3.94-3.96 (d, J=7 Hz, 1H, CH), 4.30-4.92 (m, 13H, PhCH), 5.11-5.15 (d, J=12 Hz, 1H, PhCH), 7.14-7.46 (m, 35H, ArH); Mass: m/z 1052 (M⁺+1).

### Example 15I

### Preparation of 2,3,4-Tri-O-benzyl-6-N-{2-[N-(2,6-diethylphenyl)piperazinyl]acetyl}-N-propyl-D-gluco-δ-lactam (Compound No. 62)

The title compound was prepared, and the following spectral data was used to confirm product formation: IR(CH₂Cl₂): ν 1665 cm⁻¹; ¹HNMR (CDCl₃): δ 0.90-0.95 (t, J=7 Hz, 3H, CH₃), 1.15-1.20 (t, J=7.5 Hz, 6H, 2×CH₃), 1.60-1.65 (m, 2H, CH₂), 2.49-2.54 (m, 4H, CH₃), 2.60-2.68 (q, J=7.5 Hz, 4H, 2×CH₂), 3.03-3.04 (m, 7H), 3.30 (m, 1H, CH), 3.54-3.65 (m, 4H, 4×CH), 3.72-3.76 (m, 1H, CH), 4.04-4.06 (d, J=7 Hz, 1H, CH), 4.55-4.75 (m, 5H, 5×PhCH), 5.14-5.18 (d, J=12 Hz, 1H, PhCH), 7.04-7.07 (m, 3H, ArH), 7.25-7.44 (m, 15H, ArH); Mass: m/z 747 (M⁺+1).

### Example 15J

### Preparation of 2,3,4-Tri-O-benzyl-6-{2-[1H-isoindole-1,3(2H)-diketo]acetyl}-D-gluco-δ-lactam (Compound No .63)

The title compound was prepared, and the following spectral data was used to confirm product formation: IR(CH₂Cl₂): ν 1720.7 cm⁻¹; ¹HNMR (CDCl₃): δ 0.84-0.89 (m, 3H, CH₃), 1.59 (m, 2H, CH₂), 3.01 (m, 1H, CH), 3.18 (m, 1H, CH), 3.60-3.70 (m, 4H, 4×CH), 3.88-3.89 (m, 1H, CH), 4.03-4.05 (d, J=6 Hz, 1H, CH), 4.16 (s, 2H, CH₂), 4.47-4.51 (d, J=12 Hz, 1H, PhCH), 4.57 (s, 2H, 2×PhCH), 4.67-4.75 (m, 2H, 2×PhCH), 5.11-5.15 (d, J=12 Hz, 1H, CH), 5.85 (brs, 1H, NH), 7.20-7.43 (m, 15H, ArH), 7.72-7.75 (m, 2H, ArH), 7.85-7.86 (m, 2H, ArH); Mass: m/z 676 (M⁺+1).

### Example 15K

### Preparation of 2,3,4-Tri-O-benzyl-6-N-{2-[4-(4-chlorophenyl)piperazinyl]acetyl}-N-propyl-D-gluco-δ-lactam (Compound No. 64)

The title compound was prepared, and the following spectral data was used to confirm product formation: IR: 1667cm⁻¹; ¹HNMR (CDCl₃): δ 0.87-0.91 (t, J=6 Hz, 3H, CH₃), 1.61-1.65 (m, 8H, 8×CH), 1.89 (m, 6H, 6×CH), 2.05 (m, 3H, 3×CH), 2.98-3.06 (m, 2H, 2×CH), 3.46-3.48 (m, 1H, CH), 3.66-3.76 (m, 3H, 3×CH), 3.84-3.88 (m, 1H, CH), 4.00-4.03 (m, 2H, CH+NH), 4.33-4.66 (m, 1H, NH), 4.50-4.54 (d, J=12 Hz, 1H, PhCH), 4.57-4.75 (m, 4H, 4×PhCH), 5.08-5.12 (d, J=12 Hz, 1H, PhCH), 7.21-7.43 (m, 15H, ArH); Mass: m/z 666 (M⁺+1).

### Example 15L

### Preparation of 2,3,4-Tri-O-benzyl-6-N-{2-[4-(3-(trifluoromethyl)phenyl)piperazin-1-yl]acetyl}-N-propyl-D-gluco-δ-lactam (Compound No. 65)

The title compound was prepared, and the following spectral data was used to confirm product formation: IR(CH₂Cl₂): ν 1668.5 cm⁻¹; ¹HNMR (CDCl₃): δ 0.91-0.95 (t, J=6 Hz, 3H, CH₃), 1.64-1.67 (m, 2H, CH₂), 2.45-2.51 (m, 4H, 2×CH₂), 2.91-3.05 (m, 8H, 3×CH₂+2×CH), 3.58-3.59 (m, 1H, CH), 3.73-3.75 (m, 3H, 3×CH), 3.93 (m, 1H, CH), 4.06-4.08 (d, J=6 Hz, 1H, CH), 4.52-4.67 (m, 4H, 4×PhCH), 4.67-4.71 (d, J=12 Hz, 1H, PhCH), 5.14-5.18 (d, J=12 Hz, 1H, PhCH), 6.98-7.44 (m, 19H, ArH); Mass: m/z 759 (M⁺+1).

### Example 16

### Preparation of N-propyl-6-{2-[triazolyl]-acetyl}-D-gluco-δ-lactam (Compound No. 66)

The ester obtained in Example 15 was debenzylated following the procedure described in Example 3. The product had m.p.: 210° C. The following spectral data was used to confirm product formation: IR(KBr): ν 1662, 1639 cm⁻¹; ¹HNMR (D₂O): δ 0.77-0.81 (t, J=7 Hz, 3H, CH₃), 1.41-1.51 (m, 2H, CH₂), 2.90-2.91 (m, 1H, NCH), 3.50-3.65 (m, 6H, 6×CH), 4.06-4.08 (d, 1H, CH), 5.04 (s, 2H, 2×CH), 7.61 (s, 1H, NH), 8.04 (s, 1H, TrH), 8.44 (s, 1H, TrH); Mass: m/z 328 (M⁺+1).

The following compounds were prepared analogously:

### Example 16A

### Preparation of 6-N-{2-[1,5-Dideoxy-1,5-imino-glucit-6-ol]acetyl}-N-propyl-D-gluco-δ-lactam (Compound No. 67)

The title compound was prepared, and the following spectral data was used to confirm product formation: IR(CH₂Cl₂): ν 1663 cm⁻¹; ¹HNMR (CDCl₃): δ 0.81-0.86 (t, J=7 Hz, 3H, CH₃), 1.41-1.59 (m, 2H, 2×CH), 2.38-2.42 (m, 2H, 2×CH), 2.84-2.89 (m, 2H, 2×CH), 2.94-3.01 (m, 2H, 2×CH), 3.07-3.91 (m, 10H, 10×CH), 4.00 (m, 1H, CH), 4.10 (m, 1H, CH); Mass: m/z 442 (M⁺+1).

### Example 16B

### Preparation of 6-N-{2-[(N-(Methyl)piperazinyl]acetyl}-N-propyl-D-gluco-δ-lactam (Compound No. 68)

The title compound was prepared and the following spectral data was used to confirm product formation: IR: 1650.8, 1664.4 cm⁻¹; ¹HNMR (CDCl₃+DMSO): δ 0.77-0.82 (t, J=7 Hz, 3H, CH₃), 1.41-1.48 (m, 2H, 2×CH), 1.81 (s, 3H, CH₃), 2.14-2.39 (m, 6H, 3×CH₂), 2.49 (s, 2H, CH₂), 2.89-2.93 (m, 3H, 3×CH), 3.31-3.73 (m, 7H); Mass: m/z 359 (M⁺+1).

### Example 17

### Preparation of N-allyl-2,3,4-tri-O-benzyl-6-triazolyl-D-gluco-δ-lactam (Compound No. 69)

N-Allyl-6-O-(p-toluenesulphonyl)-2,3,4-tri-O-benzyl-D-gluco-δ-lactam (1 mmol), prepared in Example 1, was dissolved in DMF. Triazole (2 mmol) and K₂CO₃ (2 mmol) were added to the solution. The reaction mixture was warmed to 60° C. and stirred for an additional 5 hours at this temperature. The contents of the reaction mixture were poured into cold water (25 ml), and extracted with ethyl acetate (2×25 ml). The organic layer was dried and the residue was evaporated using column chromatography with ethyl acetate-hexane (7:3) as eluent to yield a title compound in a semisolid state.

The following spectral data was used to confirm product formation: IR(CH₂Cl₂): ν 1672 cm⁻¹; ¹HNMR (CDCl₃): δ 3.37-3.42 (dd, J=9 Hz, 1H, CH), 3.46 (m, 1H, CH), 3.90 (m, 1H, CH), 3.98-4.00 (d, J=6 Hz, 1H, CH), 4.07 (m, 1H, CH), 4.18-4.25 (m, 2H, 2×CH), 4.34-4.39 (m, 1H, CH), 4.43-4.45 (m, 3H, 2×CH+PhCH), 4.56-4.59 (d, J=12 Hz, 1H, PhCH), 5.10-5.14 (d, J=12 Hz, 1H, PhCH), 5.18-5.27 (m, 2H, 2×PhCH), 5.70-5.76 (m, 1H, CH), 7.15-7.54 (m, 15H, ArH), 7.75 (s, 1H, TrH), 7.9 (s, 1H, TrH); Mass: m/z 539 (M⁺+1).

### Example 18

### Preparation of 6-triazolyl-N-propyl-D-gluco-δ-lactam (Compound No. 70)

The ester obtained in Example 17 was debenzylated by the procedure described in Example 3 to yield the title compound in a semisolid state. The following spectral data was used to confirm product formation: IR(CH₂Cl₂): ν 1651 cm⁻¹; ¹HNMR (CDCl₃): δ 0.71-0.76 (t, J=7.2 Hz, 3H, CH₃), 1.38-1.49 (m, 2H, 2×CH₂), 2.66-2.69 (m, 1H, CH), 3.44-3.51 (m, 2H, 2×CH), 3.69-3.77 (m, 3H, 3×CH), 4.50-4.62 (m, 2H, 2×CH), 7.99 (s, 1H, TrH), 8.37 (s, 1H, TrH); Mass: m/z 271 (M⁺+1).

### Example 19

### Preparation of N-Allyl-2,3,4-tri-O-benzyl-1,5-deoxy-1,5-imino-D-glucitol (Compound No. 71)

The ester obtained in Example 17 was reduced following the procedure described in Example 6 to yield the title compound in a semisolid state. The following spectral data was used to confirm product formation: IR(CH₂Cl₂): ν 1503, 1454 cm⁻¹; ¹HNMR (CDCl₃): δ 2.08 (m, 2H, 2×CH), 2.67-2.70 (m, 1H, CH), 3.15-3.26 (m, 3H, 3×CH), 3.41-3.58 (m, 3H, 3×CH), 4.36-4.46 (m, 2H, PhCH+CH), 4.55-4.68 (m, 4H, 4×PhCH), 4.75-4.79 (d, J=12 Hz, 1H, PhCH), 4.91-4.97 (m, 2H, 2×CH), 5.76-5.81 (m, 1H, CH), 7.22-7.32 (m, 15H, ArH), 7.92 (s, 1H, TrH), 8.17 (s, 1H, TrH); Mass: m/z 525 (M⁺+1).

### Example 20

### Preparation of 1,5-dideoxy-1,5-imino-6-triazolyl-glucitol (Compound No. 72)

The ester prepared in Example 19 (0.523 gm) was dissolved in ethanol (10 ml). 10% Palladium (0.523 gm), formic acid (5 drops) and cyclohexene (15 ml) were added to the above suspension, which was then heated to reflux temperature for 6 hours. The reaction mixture was filtered through celite, washed with methanol and added methanolic ammonia solution (5 N, 15 ml) to the mother liquor. The mother liquor was removed under vacuum. The residue was recrystallized from ethylacetate-hexane (1:5) (10 ml) to yield the pure compound to yield the title compound in a semi solid state. The following spectral data was used to confirm product formation: IR(Br): ν 1508 cm⁻¹; ¹HNMR (CDCl₃): δ 2.31-2.39 (m, 1H, CH), 2.88-2.95 (m, 1H, CH), 2.97-3.03 (m, 1H, CH), 3.09-3.15 (t, J=9 Hz, 1H, CH), 3.28-3.34 (t, J=9 Hz, 1H, CH), 3.38-3.46 (m, 1H, CH), 4.25-4.32 (m, 1H, CH), 4.54-4.62 (m, 1H, CH), 8.03 (s, 1H, TrH), 8.40 (s, 1H, TrH); Mass: m/z 215 (M⁺+1).

### Example 21

### Preparation of 1,5-dideoxy-1,5-imino-N-propyl-6-triazolyl glucitol (Compound No. 73)

The ester obtained in Example 19 (0.523 gm) was dissolved in chloroform (10 ml). Ethereal HCl (1N, 5 ml) was added to the solution at 0° C. The reaction mixture was stirred for 1 hour. Solvent was removed under reduced pressure. Pd(C) (10%, 0.523 gm) was added to the residue. The system was placed under a hydrogen atmosphere using a hydrogen balloon. Methanol (10 ml) was added to the reaction mixture and stirred for another 12 hours. Methanolic ammonia was added to the reaction mixture and it was filtered through a celite pad. The mother liquor was removed under vacuo and the residue was purified by column chromatography using methanol-ethylacetate (1:9) as the eluent mixture to yield the title compound in semisolid state.

The following spectral data was used to confirm product formation: ¹HNMR (D₂O): δ 0.72-0.76 (t, J=7.2 Hz, 3H, CH₃), 1.23-1.40 (m, 2H, CH₂), 2.15-2.23 (t, J=11 Hz, 1H, NCH), 2.55-2.76 (m, 3H, 3×CH), 2.94-2.98 (m, 1H, CH), 3.09-3.25 (m, 2H, 2×CH), 3.33-3.38 (m, 1H, CH), 4.49-4.62 (m, 2H, 2×CH), 7.94(s, 1H, TrH), 8.39 (s, 1H, TrH); Mass: m/z 257 (M⁺+1).

### Example 22

### Preparation of 2,3,4-Tri-O-benzyl-6-N-[phenyl carbamate]-N-propyl-D-gluco-δ-lactam (Compound No. 74)

The amine (1 mmol) obtained in Step 3, Example 2 was dissolved in dichloromethane (15 ml) and the reaction mixture was cooled to −60° C. Triethyl amine (2 mmol) and phenylchloroformate (1 mmol) were added to the above solution and the reaction was allowed to come to room temperature. The reaction was stirred for 2 hours at this temperature. The reaction mixture was diluted with water (25 ml). The compound was extracted with dichloromethane (2 times, 25 ml). The organic layer was dried over anhydrous Na₂SO₄, solvent distilled off under vacuo and the product was purified by column chromatography using ethyl acetate-hexane (4:6) as an eluent to yield the title compound in semiseolid state.

The following spectral data was used to confirm product formation: IR(CH₂Cl₂): ν 1738, 1658, 1491 cm⁻¹; ¹HNMR (CDCl₃): δ 0.88-0.93 (t, J=7 HZ, 3H, CH₃), 1.61-1.66 (m, 2H, CH₂), 3.01-3.05 (m, 1H, CH), 3.28-3.33 (m, 1H, CH), 3.51-3.57 (m, 1H, CH), 3.70-3.79 (m, 3H, 3×CH), 3.89-3.92 (m, 1H, CH), 4.06-4.08 (d, J=7 Hz, 1H, CH), 4.53-4.70 (m, 3H, 3×CH), 4.71-4.76 (m, 2H, 2×CH), 5.14-5.18 (d, J=9 Hz, 2H, ArH), 7.21-7.45 (m, 18H, ArH); Mass: m/z 609 (M⁺+1).

### Example 23

### Preparation of 2,3,4-Tri-O-benzyl-6-N-{4-[4-chlorophenyl]piperazine carboxamido}-N-propyl-D-gluco-δ-lactam (Compound No. 75)

Step 1: Preparation of 2,3,4-Tri-O-benzyl-6[p-nitrophenylcarbamate]-N-propyl-D-gluco-δ-lactam

The title compound was prepared following the procedure described in Example 22, using p-nitrophenylchloroformate in place of phenyl chloroformate.

Step 2: Preparation of 2,3,4-Tri-O-benzyl-6-N-{4-[4-chlorophenyl]-piperazine carboxamido}-N-propyl-D-gluco-δ-lactam

The compound prepared in Step 1 (1 mmol) was dissolved in dichloromethane (DCM) (10 ml). Triethylamine (2 mmol) and 4-chlorophenyl piperazine (1 mmol) were added to the reaction mixture and stirred at room temperature for 1 hour. The reaction mixture was diluted with water (50 ml) and the compound was extracted with dichloromethane (2 times, 50 ml). The organic layer was dried over anhydrous Na₂SO₄, solvent removed under reduced pressure and the product purified by column chromatography using ethylacetate-hexane (6:4) as an eluent mixture to yield the title compound in a semisolid state.

The following spectral data was used to confirm product formation: IR(DCM): ν 1652 cm⁻¹; ¹HNMR (CDCl₃): δ 0.88-0.93 (t, J=7 Hz, 3H, CH₃), 1.60-1.67 (m, 2H, CH₂), 2.96-2.97 (m, 4H, 2×CH₂), 3.19-3.25 (m, 6H, 2×CH₂+2×CH), 3.63-3.66 (m, 3H, 3×CH), 3.79-3.80 (m, 1H, CH), 3.91-3.93 (m, 1H, CH), 4.05-4.07 (d, J=6 Hz, 1H, CH), 4.47-4.51 (d, J=12 Hz, 1H, PhCH), 4.57 (s, 2H, 2×PhCH), 4.67-4.78 (m, 3H, 2×PhCH+NH), 5.12-5.16 (d, J=12 Hz, 1H, PhCH), 6.77-6.80 (d, J=9 Hz, 2H, ArH), 7.21-7.32 (m, 15H, ArH), 7.43-7.46 (d, J=9 Hz, 2H, ArH); Mass: m/z 711 (M⁺+1).

The following compounds were prepared analogously by using different reactants in Step 2.

### Example 23A

### Preparation of 2,3,4-Tri-O-benzyl-6-N-{4-[4-fluorophenyl]piperazinyl carboxamido}-N-propyl-D-gluco-δ-lactam (Compound No. 76)

The title compound was prepared, and the following spectral data was used to confirm product formation: IR(DCM): ν 1653.2 cm⁻¹; ¹HNMR (CDCl₃): δ 0.89-0.93 (t, J=6 Hz, 3H, CH₃), 1.59-1.65 (m, 2H, CH₂), 2.91 (m, 4H, 2×CH₂), 3.20-3.25 (m, 6H, 2×CH₂+2×CH), 3.61-3.63 (m, 3H, 3×CH) 3.66 (m, 1H, CH), 3.91-3.93 (m, 1H, CH), 4.05-4.07 (d, J=6 Hz, 1H, CH), 4.47-4.51 (d, J=12 Hz, 1H, PhCH), 4.57 (m, 2H, 2×PhCH), 4.67-4.78 (m, 3H, 2×PhCH+NH), 5.13-5.17 (d, J=12 Hz, 1H, CH), 6.81-6.85 (m, 2H, ArH), 6.96-701 (m, 2H, ArH), 7.21-7.33 (m, 13H, ArH), 7.43-7.46 (m, 2H, ArH); Mass: m/z 694 (M⁺+1).

### Example 23B

### Preparation of 2,3,4-Tri-O-benzyl-6-N-{1,2-dihydro-(2H)-indolyl carboxamido}-N-propyl-D-gluco-δ-lactam (Compound No. 77)

The title compound was prepared, and the following spectral data was used to confirm product formation: IR(DCM): ν 1658.4 cm⁻¹; ¹HNMR (CDCl₃): δ 0.90-0.94 (t, J=6 Hz, 3H, CH₃), 1.62-1.69 (m, 2H, CH₂), 2.98-3.04 (t, J=9 Hz, 2H, CH₂), 3.18-3.20 (m, 2H, 2×CH), 3.34-3.38 (m, 2H, CH₂), 3.69-3.71 (m, 1H, CH), 3.70-3.71 (m, 1H, CH), 3.93-3.94 (m, 1H, CH), 4.07-4.09 (d, J=6 Hz, 1H, CH), 4.47-4.51 (d, J=6 Hz, 1H, CH), 4.47-4.51 (d, J=12 Hz, 1H, PhCH), 4.56 (s, 2H, 2×PhCH), 4.67-4.78 (m, 3H, 2×PhCH+NH), 5.13-5.17 (d, J=6 Hz, 1H, PhCH), 6.92-6.94 (t, J=6 Hz, 1H, ArH), 7.11-7.46 (m, 17H, ArH), 7.81-7.83 (d, J=6 Hz, 1H, ArH); Mass: m/z 634 (M⁺+1).

### Example 23C

### Preparation of 2,3,4-Tri-O-benzyl-6-N-{3-(2-iminocarbonylaminoethyl)indolyl}-N-propyl-D-gluco-δ-lactam (Compound No. 78)

The title compound was prepared, and the following spectral data was used to confirm product formation: IR(DCM): ν 1644.7 cm⁻¹; ¹HNMR (CDCl₃): δ 0.85-0.90 (m, 3H, CH₃), 1.55-1.57 (m, 2H, CH₂), 2.88-2.92 (m, 3H, CH₂₊CH), 3.15-3.25 (m, 1H, CH), 3.42-3.48 (m, 3H, 3×CH), 3.63-3.64 (m, 2H, 2×CH), 3.65-3.75 (m, 1H, CH), 3.80-3.85 (m, 1H, CH), 3.95-3.97 (m, 1H, CH), 4.52-4.74 (m, 5H, 5×PhCH), 5.04-5.08 (d, J=12 Hz, 1H, PhCH), 6.94-7.54 (m, 19H, ArH), 8.05 (s, 1H, NH); Mass: m/z 675 (M⁺+1).

### Example 23D

### Preparation of 2,3,4-Tri-O-benzyl-6-N-{(1α,5α,6α)-6-acetylamino-azabicyclo[3.1.0]hexyl carboxamido}-N-propyl-D-gluco-δ-lactam (Compound No. 79)

The title compound was prepared, and the following spectral data was used to confirm product formation: IR(DCM): ν 1656 cm⁻¹; ¹HNMR (CDCl₃): δ 0.89-0.94 (t, J=6 Hz, 3H, CH₃), 1.60-1.65 (m, 4H, 4×CH), 2.09 (s, 3H, CH₃), 2.36 (s, 1H, CH), 3.09-3.11 (m, 2H, 2×CH), 3.22-3.25 (m, 2H, 2×CH), 3.45-3.46 (m, 1H, CH), 3.56-3.75 (m, 4H, 4×CH), 3.90-3.92 (m, 2H, 2×CH), 4.04-4.07 (d, J=9 Hz, 1H, CH), 4.46-4.76 (m, 6H, 5×PhCH+NH), 5.13-5.17 (d, J=12 Hz, 1H, PhCH), 5.69 (s, 1H, NH), 7.20-7.47 (m, 15H, ArH); Mass: m/z 655 (M⁺+1).

### Example 24

### Preparation of N-{4-[phenyl]piperazinyl carboxamido}-D-gluco-δ-lactam (Compound No. 80)

The ester obtained in Example 23 was debenzylated following the procedure described in Example 3 to yield the title compound to yield the title compound in a semisolid state. The following spectral data was used to confirm product formation: IR(DCM): ν 1644.6 cm⁻¹; ¹HNMR (CDCl₃): δ 0.82-0.87 (t, J=6 Hz, 3H, CH₃), 1.48-1.57 (m, 2H, 2×CH), 3.00 (m, 5H, 5×CH), 3.33-3.50 (m, 6H, 6×CH), 3.70-3.74 (m, 4H, 4×CH), 3.96 (s, 1H, CH), 6.76-6.89 (m, 2H, ArH), 7.16-7.25 (m, 3H, ArH); Mass: m/z 406 (M⁺+1).

### Example 25

### Preparation of 2,3,4-tri-O-benzyl-6-[4-chlorophenyl-3(2H,4H)-1,2,4-triazol-3-onyl]-N-allyl-D-gluco-δ-lactam (Compound No. 81)

N-Allyl-2,3,4-tri-O-benzyl-D-gluco-δ-lactam (1 mmol), triphenyl phosphine (PPh₃) (2 mmol) and (4chlorophenyl)-2,4-dihydro-3H-1,2,4-triazol-3-one (1.1 mmol) were dissolved in tetrahydrofuran (THF) (20 ml) and the reaction mixture was cooled to 0° C. Diisobutyl azadicarboxylate (DIAD) (2 mmol) was added dropwise and the reaction was allowed to come to room temperature and stirred for 12 hours. THF was distilled off. The residue was diluted with water (50 ml) and extracted with ethylacetate (2 times 50 ml). The organic layer was dried over anhydrous Na₂SO₄, solvent removed under vaccuo and the compound was purified by column chromatography using ethylacetate-hexane (3:7) as an eluent mixture to yield the title compound in a semisolid state.

The following spectral data was used to confirm product formation: R(DCM): ν 1713.5, 1671 cm⁻¹; ¹HNMR (CDCl₃): δ 3.49-3.54 (m, 1H, CH), 3.88-3.94 (m, 3H, 3×CH), 4.05-4.09 (m, 2H, 2×CH), 4.12-4.14 (d, J=6 Hz, 1H, CH), 4.51-4.79 (m, 6H, 6×PhCH), 5.14-5.26 (m, 3H, 3×CH), 5.73 (m, 1H, CH), 7.28-7.46 (m, 19H, ArH), 7.59 (s, 1H, ArH); Mass: m/z 665 (M⁺+1).

The following compounds were prepared analogously, as will be recognized by one of ordinary skill in the art:

### Example 25A

### Preparation of N-Allyl-2,3,4-tri-O-benzyl-6-(2,6-diketopiperidino)-D-gluco-δ-lactam (Compound No. 82)

The title compound was prepared, and the following spectral data was used to confirm product formation: IR(DCM): ν 1702.3, 1675.3 cm⁻¹; ¹HNMR (CDCl₃): δ 1.98-2.02 (t, J=6 Hz, 2H, CH₂), 2.60-2.64 (t, J=6 Hz, 4H, 2×CH₂), 3.44-3.46 (m, 1H, CH), 3.67 (s, 1H, CH), 3.74-3.92 (m, 3H, 3×CH), 4.20-4.24 (m, 1H, CH), 4.34-4.44 (m, 3H, 3×CH), 4.51-4.56 (m, 2H, 2×CH), 4.69-4.72 (d, J=9 Hz, 2H, 2×CH), 5.10-5.18 (m, 3H, 3×CH), 5.56-5.64 (m, 1H, CH), 7.22-7.32 (m, 13H, ArH), 7.47-7.50 (m, 2H, ArH); Mass: m/z 583 (M⁺+1).

### Example 25B

### Preparation of N-Allyl-2,3,4tri-O-benzyl-6-(1H-isoindole-1,3-(2H)-diketo)-D-gluco-δ-lactam (Compound No. 83)

The title compound was prepared, and exhibited an m.p.: 113-114° C. The following spectral data was used to confirm product formation: IR(DCM): ν 1715.3, 1676 cm⁻¹; ¹HNMR (CDCl₃): δ 3.56-3.59 (dd, 1H, CH), 3.74-3.76 (m, 2H, 2×CH), 3.83-3.91 (m, 2H, 2×CH), 3.97 (m, 1H, CH), 4.40-4.55 (dd, 1H, CH), 4.45-4.47 (m, 3H, 2×CH), 4.59-4.63 (d, J=12 Hz, 1H, PhCH), 4.69-4.73 (m, 2H, 2×CH), 4.91-4.95 (d, J=12 Hz, 1H, PhCH), 5.06-5.16 (m, 2H, 2×CH), 5.56-5.64 (m, 1H, CH), 7.17-7.36 (m, 13H, ArH), 7.48-7.50 (m, 2H, ArH), 7.74-7.77 (m, 2H, ArH), 7.83-7.87 (m, 2H, ArH); Mass: m/z 617 (M⁺+1).

### Example 25C

### Preparation of N-Allyl-2,3,4-tri-O-benzyl-6-(2,5-diketopyrrolidino)-D-gluco-δ-lactam (Compound No. 84)

The title compound was prepared, and the following spectral data was used to confirm product formation: IR(DCM): ν 1704.5, 1676.7 cm⁻¹; ¹HNMR (CDCl₃): δ 2.61 (s, 4H, 2×CH₂), 3.58-3.67 (m, 3H, 3×CH), 3.85-3.88 (m, 3H, 3×CH), 4.10-4.25 (m, 1H, CH), 4.38-4.43 (m, 2H, 2×CH), 4.46 (m, 1H, CH), 4.56-4.60 (m, 1H, CH), 4.67-4.73 (m, 2H, 2×CH), 5.10-5.17 (m, 3H, 3×CH), 5.65-5.75 (m, 1H, CH), 7.20-7.33 (m, 13H, ArH), 7.46-7.49 (m, 2H, ArH); mass: m/s/569 (M⁺+1).

### Example 26

### Preparation of N-allyl-2,3,4-tri-O-benzyl-1,5-dideoxy-1,5-imino-6-morpholino glucitol (Compound No. 85)

The ester obtained in Example 25 was reduced following the procedure described in Example 6 to give the title compound. The title compound exhibited an m.p.: 71° C., and the following spectral data was used to confirm product formation: IR (CHCl₃): ν 1496, 1453 cm⁻¹; ¹HNMR (CDCl₃): δ 2.00-2.04 (m, 1H, CH), 2.26-2.29 (m, 2H, CH₂), 2.35 (m, 1H, CH), 2.54-2.62 (m, 4H, 4×CH), 2.82-3.00 (m, 2H, 2×CH), 3.42-3.65 (m, 8H, 8×CH), 4.64-4.68 (m, 3H, 3×PhCH), 4.76-4.80 (d, J=12 Hz, 1H, 3×PhCH), 4.95-5.00 (m, 2H, 2×PhCH), 5.12-5.19 (m, 2H, 2×CH), 5.82-5.84 (m, 1H, CH), 7.25-7.32 (m, 15H, ArH); Mass: m/s 543 (M⁺+1).

### Example 27

### Preparation of 6-(2,5-Diketopyrrolidino)-N-propyl-D-gluco-δ-lactam (Compound No. 86)

The ester obtained in Example 25 was debenzylated by the procedure described in Example 3, to yield the title compound in a semisolid state. The following spectral data was used to confirm product formation: ¹HNMR (CD₃OD): δ 0.78-0.82 (t, J=6 Hz, CH₃), 1.46-1.50 (m, 2H, 2×CH), 2.63 (s, 4H, 2×CH₂), 2.81 (m, 1H, CH), 3.33-3.38 (m, 1H, CH), 3.47-3.49 (m, 1H, CH), 3.63-3.68 (m, 4H, 4×CH), 3.91-3.94 (d, J=9 Hz, 1H, CH); Mass: m/z 301 (M⁺+1).

The following compounds were prepared analogously, as will be understood by one of ordinary skill in the art.

### Example 27A

### Preparation of 6-(2,6-Diketopiperidino)-N-propyl-D-gluco-δ-lactam (Compound No. 87)

The title compound was prepared, and exhibited an m.p.: 146-147.8° C. The following spectral data was used to confirm product formation: IR(DCM): ν 1722.7, 1660.2 cm⁻¹; ¹HNMR (D₂O): δ 0.87.0.91 (t, J=6 Hz, 3H, CH₃), 1.49-1.65 (m, 2H, 2×CH), 1.99-2.10 (m, 2H, CH₂), 2.77-2.88 (m, 5H, CH+2×CH₂), 2.77-2.88 (m, 5H, CH+2×CH₂), 3.57-3.62 (m, 1H, CH), 3.68-3.78 (m, 2H, 2×CH), 3.87-3.90 (m, 1H, CH), 4.07-4.09 (d, J=6 Hz, 2H, 2×CH), 4.30-4.33 (d, J=9 Hz, 1H, CH); Mass: m/z 315 (M⁺+1).

### Example 27B

### Preparation of N-Propyl-6-[4-chlorophenyl-3(2H,4H)-1,2,4-triazol-3-onyl]-D-gluco-δ-lactam (Compound No. 88)

The title compound was prepared, and exhibited an m.p.: 144-145.8° C. The following spectral data was used to confirm product formation: IR(KBr) : ν 1715, 1644 cm⁻¹; ¹HNMR (D₂O): δ 0.85-0.90 (t, J=6 Hz, 3H, CH₃), 1.52-1.61 (m, 2H, CH₂), 2.90-2.94 (m, 1H, CH), 3.60-3.66 (m, 1H, CH), 3.71-3.79 (m, 2H, 2×CH), 3.90-3.93 (d, J=9 Hz, 1H, CH), 4.01-4.05 (m, 1H, CH), 4.18-4.30 (m, 2H, 2×CH), 7.50-7.61 (m, 5H, ArH), 8.19 (s, 1H, TrH); Mass: m/z 363 (M⁺+1).

### Example 28

### Peparation of 2,3,4-Tri-O-benzyl-6-(4,6-dichloro-1,3,5-triazin-1-yl)-N-propyl-D-gluco-δ-lactam (Compound No. 89)

6-Amino-2,3,4-tri-O-benzyl-N-propyl-D-gluco-δ-lactam (1 mmol), prepared as described in Step 3, Example 2 was dissolved in acetone. The reaction mixture was cooled to O° C. Cyanuric chloride (1 mmol) and K₂CO₃ (6 mmol) were added to the reaction mixture. The reaction was allowed to proceed at 0° C. for 1 hour. The cooled solution was filtered and acetone removed by distillation. The residue was diluted with water (25 ml) and extracted with ethyl acetate (2 times, 25 ml). The organic layer was dried and the solvent was removed under reduced pressure. The product was purified by column chromatography using ethylacetate-hexane (3:7) as eluent to yield the title compound in a semisolid state.

The following spectral data was used to confirm product formation: IR (CH₂Cl₂): ν 1661, 1603, 1552 cm⁻¹; ¹HNMR (CDCl₃): δ 0.90-0.95 (t, J=7 Hz, 3H, CH₃), 1.57-1.64 (m, 2H, 2×CH), 3.05 (m, 1H, CH), 3.50 (m, 1H, CH), 3.58-3.65 (m, 4H, 4×CH), 3.80 (m, 1H, CH), 4.04-4.06 (d, J=6 Hz, 1H, CH), 4.53-4.57 (d, J=12 Hz, 1H, PhCH), 4.59-4.63 (d, J=12 Hz, 1H, PhCH), 4.70-4.75 (m, 3H, 3×PhCH), 5.12-5.16 (d, J=12 Hz, 1H, PhCH), 5.90 (m, 1H, NH), 7.19-7.45 (m, 15H, ArH); Mass: m/z 636 (M⁺+1).

### Example 29

### Preparation of 2,3,4-tri-O-benzyl-6-O-(4,6-dichloro-1,3,5-triazin-1-yl)-N-propyl-D-gluco-δ-lactam (Compound No. 90)

The title compound was prepared following the procedure described in Example 27, using N-allyl-2,3,4-tri-O-benzyl-D-gluco-δ-lactam instead of the amine to yield the title compound in a semisolid state. The following spectral data was used to confirm product formation: IR(CH₂Cl₂): ν 1672 cm⁻¹; ¹HNMR (CDCl₃): δ 3.70-3.75 (m, 2H, 2×CH), 3.85-3.91 (m, 2H, 2×CH), 4.07-4.12 (m, 2H, CH), 4.35-4.37 (m, 2H, 2×CH), 4.47-4.49 (m, 2H, PhCH), 4.55-4.79 (m, 4H, 4×PhCH), 5.14-5.19 (m, 1H, 2×CH), 5.24 (s, 1H, CH), 5.68-5.79 (m, 1H, CH), 7.13-7.46 (m, 15H, ArH); Mass: m/z 636 (M⁺+).

### Example 30

### Pharmacological Testing

Each agent was tested over a broad concentration range (ten-fold dilutions starting from ≧100 μM to 10 nM) against 6 human cancer cell lines which was comprised of different tumor types. These are DU145 (prostate carcinoma), HT29 (colorectal adenocarcinoma), LOX (melanoma), MCF7 (breast adenocarcinoma), MCF7ADR (adriamycin resistant breast adenocarcinoma), and U251 (human glioblastoma). A standard compound doxorubicin was tested in each assay as a positive control. The cells were maintained in growing condition in RPMI 1640 medium containing 10% fetal calf serum and incubated at 37° C. under 5% CO₂ atmosphere. All cell lines were inoculated onto a series of standard 96-well microtitre plate on day zero, followed by twenty four-hour incubation in the absence of test compound. The inoculation densities in the screen were as per Monk et al., (1991) J. Natl. Cancer Inst., 83, 757-766. All NCEs were dissolved in DMSO and diluted further in culture medium. An aliquot of each dilution was added to the growing cells in 96 well plates and incubated for 48 hours. After incubation, the assay was terminated by adding 50 μL of trichloroacetic acid (TCA) and incubating at 4° C. for 30 min. The precipitated cells were washed and stained with sulphorhodamine B dye for 30 min and the excess dye was washed off with acetic acid. Adsorbed dye was solubilised in Tris base (alkaline pH) and quantitated by measuring the OD at 490 mm in ELISA READER. GI₅₀ (concentration which inhibits the cell growth by 50%) was calculated according to Boyd and Paull, (1995) Drug Dev. Res., 34, 91-109.

In the experiments described in Table I, doxorubicin was used as a standard compound.

TABLE I
In Vitro Screening Data

| | | | | | MCF7ADR* | U251* |
|---|---|---|---|---|---|---|
| | DU145* | HT29* | | MCF7* | #Adriamycin | #Human |
| Compd. | #Prostate | #Colorectal | LOX* | #Breast | Resistant Breast | glioblastoma |
| No. | carcinoma | adenocarcinoma | #Melanoma | adenocarcinoma | adenocarcinoma | astrocytoma |
| 1 | 364.5 | 203.3 | >624 | 378.0 | >624 | >624 |
| 41 | 141.6 | 71.2 | >663.5 | 108.8 | >663.5 | >663.5 |
| 45 | 148.3 | 92.9 | >418.8 | 397.1 | >418.8 | >418.8 |
| 69 | 51.0 | 31.6 | 47.4 | 47.8 | 39.4 | 37.9 |
| 46 | 83.8 | 3.3 | 177.6 | 18.4 | 83.4 | 105.0 |
| 42 | 174.6 | 103.0 | >372.6 | 232.0 | >372.6 | >372.6 |
| 2 | 150.8 | 137.4 | 40.9 | 175.0 | 229.0 | 171.6 |
| 51 | 43.7 | 6.8 | 13.6 | 9.9 | 16.0 | 10.3 |
| 71 | 14.5 | 3.5 | 9.5 | 5.4 | 9.2 | 8.3 |
| 43 | 225.6 | 207.8 | 161.4 | 114.7 | 280.4 | 234.7 |
| 3 | 2.0 | 6.1 | 2.1 | 0.8 | 8.2 | 1.1 |
| 24 | 295.5 | 213.0 | 169.0 | 156.8 | 262.4 | 230.7 |
| 52 | 7.2 | 6.8 | 0.9 | 6.0 | 0.8 | 2.9 |
| 70 | 251.5 | 302.0 | 232.3 | 339.9 | 893.5 | 515.6 |
| 4 | 116.5 | 49.4 | 112.4 | 13.6 | 115.0 | 16.8 |
| 5 | 29.6 | 14.6 | 10.4 | 11.4 | 33.9 | 46.9 |
| 6 | 50.1 | 47.2 | 67.4 | 433.1 | 110.1 | 82.3 |
| 7 | >352.6 | 68.6 | 10.8 | >352.6 | 58.4 | 63.5 |
| 25 | 386.0 | 311.1 | 94.6 | 190.3 | 228.2 | 278.4 |
| 26 | 278.0 | 209.3 | 116.2 | 136.0 | 234.8 | 520.2 |
| 27 | 412.9 | 231.9 | 193.3 | 213.6 | 480.6 | 323.4 |
| 28 | 288.1 | 241.7 | 135.8 | 106.5 | 194.0 | 556.8 |
| 72 | 340.3 | 243.9 | 146.5 | 208.5 | 275.0 | 282.3 |
| 47 | 152.8 | 49.3 | 51.4 | 14.7 | 68.2 | 154.7 |
| 49 | 352.0 | 131.3 | 133.2 | 176.6 | 250.8 | 122.2 |
| 73 | 266.6 | 469.6 | 247.2 | 319.9 | 290.6 | 505.6 |
| 44 | 88.5 | 29.2 | 8.7 | 2.3 | 7.5 | 29.8 |
| 8 | 3.4 | 9.0 | 2.2 | 1.3 | 0.6 | 2.5 |
| 53 | 8.8 | 9.3 | 8.3 | 6.6 | 10.0 | 12.9 |
| 66 | 82.9 | 149.4 | 58.2 | 124.1 | 195.6 | 142.4 |
| 85 | 43.5 | 11.3 | 27.3 | 7.2 | 40.3 | 25.9 |
| 9 | 22.1 | 40.0 | 6.9 | 35.8 | 13.9 | 20.2 |
| 10 | 39.3 | 36.9 | 12.5 | 34.7 | 16.7 | 16.4 |
| 11 | 2.7 | 2.7 | 2.1 | 2.4 | 2.6 | 3.2 |
| 12 | 48.7 | 29.3 | 41.6 | 88.2 | 35.0 | 61.8 |
| 13 | 4.2 | 3.6 | 3.1 | 6.4 | 4.6 | 6.4 |
| 14 | 21.0 | 30.4 | 7.6 | 35.7 | 6.7 | 15.0 |
| 36 | 16.7 | 0.8 | 0.9 | 0.3 | 19.1 | 11.7 |
| 15 | 44.4 | 41.9 | 57.2 | 29.9 | 53.8 | 40.8 |
| 16 | 59.7 | 58.2 | 53.3 | 55.6 | 74.1 | 22.9 |
| 37 | 11.1 | 2.8 | 5.1 | 10.3 | 17.3 | 15.4 |
| 17 | 82.2 | 212.9 | 170.5 | >528.2 | 348.0 | 205.5 |
| 18 | 2.5 | 2.2 | 2.0 | 2.0 | 2.9 | 2.6 |
| 19 | >591.7 | >591.7 | 81.8 | >591.7 | >591.7 | >591.7 |
| 38 | 98.4 | 35.0 | 281.2 | >380.2 | >380.2 | >380.2 |
| 20 | 15.3 | 2.6 | 5.9 | 4.1 | 5.5 | 3.3 |
| 21 | 6.5 | 2.0 | 2.9 | 2.4 | 3.4 | 3.6 |
| 32 | 55.3 | 37.7 | 87.4 | 74.8 | 94.4 | 62.0 |
| 33 | 52.7 | 24.1 | 25.1 | 34.1 | 56.8 | 47.1 |
| 39 | 72.7 | 20.6 | 102.7 | 16.1 | 52.7 | 31.9 |
| 40 | 35.7 | 2.0 | 27.2 | 9.7 | 18.4 | 20.1 |
| 29 | 280.1 | 58.6 | 290.2 | 85.8 | 165.1 | 87.0 |
| 54 | 5.0 | 3.6 | 2.3 | 4.1 | 6.6 | 3.2 |
| 34 | 78.2 | 38.4 | 129.5 | 26.1 | 63.4 | 28.3 |
| 22 | 5.1 | 11.9 | 4.3 | 39.8 | 8.2 | 5.1 |
| 30 | 118.6 | 42.6 | 226.0 | 180.5 | 208.8 | 100.3 |
| 74 | 90.9 | 10.7 | 51.8 | 25.4 | 26.1 | 34.2 |
| 31 | 112.9 | 79.5 | 131.7 | 138.8 | 131.0 | 82.2 |
| 35 | 14.0 | 19.4 | 8.0 | 7.6 | 9.3 | 5.8 |
| 48 | 103.7 | 67.9 | 31.8 | 18.8 | 58.4 | 12.9 |
| 50 | 107.9 | 38.1 | 97.2 | 82.8 | 138.1 | 143.7 |
| 89 | 14.1 | 10.9 | 3.0 | 4.3 | 23.2 | 22.4 |
| 55 | 4.1 | 4.7 | 3.3 | 8.2 | 10.2 | 4.1 |
| 56 | 10.7 | 22.8 | 12.0 | 40.3 | 24.5 | 40.5 |
| 57 | 64.4 | 22.7 | 25.1 | 20.1 | 246.9 | 154.4 |
| 58 | 19.4 | 7.9 | 2.8 | 8.2 | 10.2 | 13.4 |
| 59 | 12.2 | 1.9 | 3.8 | 8.1 | 25.3 | 12.6 |
| 60 | 107.9 | 258.0 | 157.5 | 100.6 | 281.4 | 190.2 |
| 61 | 194.7 | >618.5 | >618.5 | >618.5 | >618.5 | >618.5 |
| 62 | 24.3 | 24.9 | 37.7 | >402.1 | >402.1 | 30.0 |
| 90 | 16.7 | 37.1 | 14.4 | 35.4 | 18.7 | 52.8 |
| 67 | 81.6 | 105.6 | 67.6 | 106.9 | 75.4 | 91.5 |
| 68 | 39.0 | 14.1 | 48.2 | 32.4 | 27.7 | 22.0 |
| 63 | 60.6 | 42.0 | 55.3 | 66.8 | 245.9 | 116.6 |
| 76 | 45.2 | 28.6 | 40.9 | 38.6 | 90.7 | 25.7 |
| 77 | 49.4 | 17.5 | 29.5 | 56.1 | 69.1 | 28.6 |
| 75 | 25.7 | 18.4 | 16.9 | 1.6 | 11.6 | 8.8 |
| 64 | 12.5 | 8.1 | 6.3 | 1.5 | 4.3 | 3.7 |
| 23 | 50.0 | 28.7 | 35.7 | 5.6 | 26.0 | 4.2 |
| 65 | 13.1 | 5.6 | 4.0 | 2.0 | 3.5 | 3.4 |
| 78 | 57.3 | 33.6 | 45.1 | 9.2 | 29.8 | 4.3 |
| 81 | 70.1 | 49.5 | 11.9 | 102.3 | 77.0 | >250 |
| 80 | 90.7 | 100.0 | 75.9 | 69.4 | 60.1 | 59.9 |
| 79 | 18.9 | 21.8 | 7.4 | 18.2 | 17.9 | 15.5 |
| 82 | 24.8 | 23.5 | 8.5 | 9.9 | 17.5 | 12.5 |
| 86 | 38.5 | 44.4 | 37.5 | 36.2 | 52.2 | 34.4 |
| 83 | 55.2 | 80.1 | 51.4 | 13.1 | 6.7 | >140.4 |
| 84 | 18.3 | 18.7 | 5.9 | 5.3 | 6.4 | 9.3 |
| 87 | 131.3 | 107.6 | 244.5 | 74.2 | 96.9 | 101.4 |
| 88 | 144.3 | 110.8 | 135.4 | 101.6 | 107.3 | 90.7 |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A compound having the structure of Formula I: 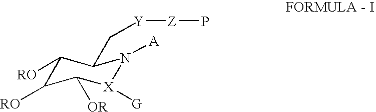
and its pharmaceutically acceptable salts, esters, enantiomers, diastereomers, N-oxides, prodrugs, metabolites, polymorphs or pharmaceutically acceptable solvates, wherein
A represents hydrogen, lower alkyl (C₁-C₄), lower alkenyl (C₁-C₄), or lower alkynyl (C₁-C₄);
X-G represents CO or CH₂;
R represents hydrogen, alkyl (C₁-C₄), acyl, aryl, aralkyl or trimethylsilyl;
Y represents O, NH or 5 to 6 membered cyclic ring optionally having one or more heteroatoms selected from the group N, O and S; 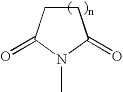
where n represents 0, 1 or 2; 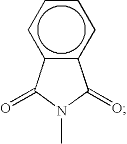 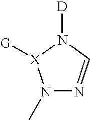
wherein X-G represents CO or CH₂, D represents an aryl group optionally substituted with F, Cl, Br and I;
Z represents CO, CS, SO₂, 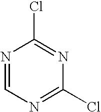
or no atom;
P represents no atom or straight or branched lower alkyl (C₁-C₄) which may be substituted with halogen selected from the group F, Cl, Br, I; trifluoromethyl; aryl which may be substitutted with one or more substituents selected from the group consisting of lower alkyl (C₁-C₃), halogen (F, Cl, Br, I); aralkyl, 5 to 6 membered heterocyclic ring with one or more hetero atoms selected from the group N, O and S; alkylamino in which the alkyl ring may be straight or branched; 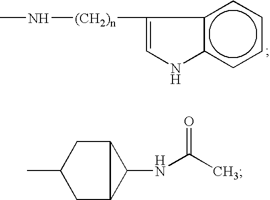
-E-U-K wherein E represents O or NH; U represents straight or branched lower alkyl (C₁-C₄) sulfonyl, adamantane, fused aryl rings or no atom; K represents 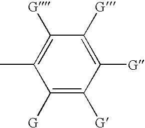
where G, G′, G″, G′″ and G″″ may be independently selected from hydrogen, lower alkyl (C₁-C₄), aryl which may optionally be substituted with one or more halogens selected from the group F, Cl, Br and I, CH₃, CF₃ OCH₃, COCH₃, NH₂ or NO₂;
—CH₂-L where L represents 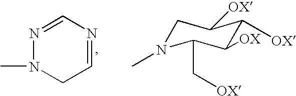
wherein X′ may be hydrogen, aryl or aralkyl; 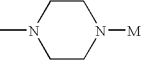
wherein M represents lower alkyl (C₁-C₄); pyrimidyl; aryl which may optionally be substituted with lower alkyl (C₁-C₃), trifluoromethyl or halogen which may be selected from the group F, Cl, Br and I; aralkyl; 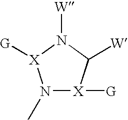
wherein X-G represents CO or CH₂, W′ W″ may independently be selected from hydrogen or may form a fused aryl ring of 6 carbon atoms; 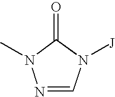
wherein J represents 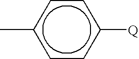
where Q represents halogen which may be selected from the group F, Cl, Br and I; or 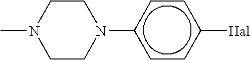
wherein Hal may be selected from the group F, Cl, Br and I; and

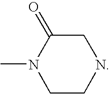

2. A compound selected from the group consisting of:
N-Allyl-6-O-(p-toluenesulphonyl)-2,3,4-tri-O-benzyl-D-gluco-δ-lactam (Compound No. 1);
2,3-4-Tri-O-benzyl-6-N-{[2-naphthyl]aminothiocarbonylamino}-N-propyl-D-gluco-δ-lactam (Compound No. 2);
2,3,4-Tri-O-benzyl-6-N-{[4-chlorophenyl]aminocarbonylamino}-N-propyl-D-gluco-δ-lactam (Compound No. 3);
2,3,4-Tri-O-benzyl-6-N-{[p-methoxyphenyl]aminocarbonylamino}-N-propyl-D-gluco-δ-lactam (Compound No. 4);
2,3,4-Tri-O-benzyl-6-N-{[p-nitrophenyl]aminocarbonylamino}-N-propyl-D-gluco-δ-lactam (Compound No. 5);
2,3,4-Tri-O-benzyl-6-N-{[p-tolyl]aminocarbonylamino}-N-propyl-D-gluco-δ-lactam (Compound No. 6);
2,3,4-Tri-O-benzyl-6-N-{[4-chloro-2-trifluoromethylphenyl]aminocarbonylamino}-N-propyl-D-gluco-δ-lactam (Compound No. 7);
2,3,4-Tri-O-benzyl-6-N-{[isopropyl]aminothiocarbonylamino}-N-propyl-D-gluco-δ-lactam (Compound No. 8);
2,3,4-Tri-O-benzyl-6-N-{[phenyl]aminothiocarbonylamino}-N-propyl-D-gluco-δ-lactam (Compound No. 9);
2,3,4-Tri-O-benzyl-6-N-{[phenyl]aminocarbonylamino}-N-propyl-D-gluco-δ-lactam (Compound No. 10);
2,3,4-Tri-O-benzyl-6-N-{[p-fluorophenyl]aminocarbonylamino}-N-propyl-D-gluco-δ-lactam (Compound No. 11);
2,3,4-Tri-O-benzyl-6-N-{[p-chlorophenylsulfonyl]aminocarbonylamino}-N-propyl-D-gluco-δ-lactam (Compound No. 12);
2,3,4-Tri-O-benzyl-6-N-{[3-chlorophenyl]aminocarbonylamino}-N-propyl-D-gluco-δ-lactam (Compound No. 13);
2,3,4-Tri-O-benzyl-6-N-{[3-acetylphenyl]aminocarbonylamino}-N-propyl-D-gluco-δ-lactam (Compound No. 14);
2,3,4-Tri-O-benzyl-6-N-{[3-chloro-6-methoxyphenyl]aminocarbonylamino}-N-propyl-D-gluco-δ-lactam (Compound No. 15);
2,3,4-Tri-O-benzyl-6-N-{[2,4-difluorophenyl]aminocarbonylamino}-N-propyl-D-gluco-δ-lactam (Compound No. 16);
2,3,4-Tri-O-benzyl-6-N-{[2,4,6-trichlorophenyl]aminocarbonylamino}-N-propyl-D-gluco-δ-lactam (Compound No. 17);
2,3,4-Tri-O-benzyl-6-N-{[3,4-dichlorophenyl]aminocarbonylamino}-N-propyl-D-gluco-δ-lactam (Compound No. 18);
2,3,4-Tri-O-benzyl-6-N-{[2,4-dichlorophenyl]aminocarbonylamino}-N-propyl-D-gluco-δ-lactam (Compound No. 19);
2,3,4-Tri-O-benzyl-6-N-{[isopropyl]aminocarbonylamino}-N-propyl-D-gluco-δ-lactam (Compound No. 20);
2,3,4-Tri-O-benzyl-6-N-{[4-chlorophenyl]aminothiocarbonylamino}-N-propyl-D-gluco-δ-lactam (Compound No. 21);
2,3,4-Tri-O-benzyl-6-N-{[4-trifluoromethylphenyl]aminocarbonylamino}-N-propyl-D-gluco-δ-lactam (Compound No. 22);
2,3,4-Tri-O-benzyl-6-N-{[1-adamantyl]aminocarbonylamino}-N-propyl-D-gluco-δ-lactam (Compound No. 23);
N-Propyl-6-N-{[phenyl]aminocarbonylamino}-D-gluco-δ-lactam (Compound No. 24);
6-N-{[2-Trifluoromethylphenyl]aminocarbonylamino}-N-propyl-D-gluco-δ-lactam (Compound No. 25);
6-N-{[p-Tolyl]aminocarbonylamino}-N-propyl-D-gluco-δ-lactam (Compound No. 26);
6-N-{[p-Methoxypheny]aminocarbonylamino}-N-propyl-D-gluco-δ-lactam (Compound No. 27);
6-N-{[p-Aminophenyl]aminocarbonylamino}-N-propyl-D-gluco-δ-lactam (Compound No. 28);
6-N-{[4-Fluorophenyl]aminocarbonylamino}-N-propyl-D-gluco-δ-lactam (Compound No. 29);
6-N-{[Isopropyl]aminocarbonylamino}-N-propyl-D-gluco-δ-lactam (Compound No. 30);
6-N-{[4-Trifluoromethylphenyl]aminocarbonylamino}-N-propyl-D-gluco-δ-lactam (Compound No. 31);
2,3,4-Tri-O-acetyl-6-N-{[4-methoxyphenyl]aminocarbonylamino}-N-propyl-D-gluco-δ-lactam (Compound No. 32);
2,3,4-Tri-O-acetyl-6-N-{[phenyl]aminocarbonylamino}-N-propyl-D-gluco-δ-lactam (Compound No. 33);
2,3,4-Tri-O-acetyl-6-N-{[4-fluorophenyl]aminocarbonylamino}-N-propyl-D-gluco-δ-lactam (Compound No. 34);
2,3,4-Tri-O-trimethylsilyl-6-N-{[phenyl]aminocarbonylamino}-N-propyl-D-gluco-δ-lactam (Compound No. 35);
2,3,4-Tri-O-benzyl-6-N-{[4-chlorophenyl]aminocarbonylamino}-1,5-dideoxy-1,5-imino glucitol (Compound No. 36);
2,3,4-Tri-O-benzyl-6-N-{[2,4-difluorophenyl]aminocarbonylamino}-1,5-dideoxy-1,5-imino glucitol (Compound No. 37);
2,3,4-Tri-O-benzyl-6-N-{[isopropyl]aminocarbonylamino}-1,5-dideoxy-1,5-imino-N-propyl glucitol (Compound No. 38);
2,3,4-Tri-O-benzyl-6-N-{[4-chlorophenyl]aminothiocarbonylamino}-1,5-dideoxy-1,5-imino-N-propyl glucitol (Compound No. 39);
2,3,4-Tri-O-benzyl-6-N-{[4-fluorophenyl]aminocarbonylamino}-1,5-dideoxy-1,5-imino-N-propyl glucitol (Compound No. 40);
N-Allyl-6-O-(4-chlorophenylcarbamate)-2,3,4-tri-O-benzyl-D-gluco-δ-lactam (Compound No. 41);
N-Allyl-2,3,4-Tri-O-benzyl-6-(2-naphthylthlocarbamate)-D-gluco-δ-lactam (Compound No. 42);
N-Propyl-6-(phenylcarbamate)-N-propyl-D-gluco-δ-lactam (Compound No. 43);
N-Allyl-2,3,4-tri-O-benzyl-6-{p-chlorophenylcarbamate}-1,5-dideoxy-1,5-imino glucitol (Compound No. 44);
N-allyl-6-O-(2-thiophenyl)-2,3-4-tri-O-benzyl-D-gluco-δ-lactam (Compound No. 45);
2,3,4-Tri-O-benzyl-N-propyl-6-{[2-thiophene]carboxamido}-D-gluco-δ-lactam (Compound No. 46);
2,3,4-Tri-O-benzyl-6-N-{[2,4-difluorophenyl]carboxamido}-N-propyl-D-gluco-δ-lactam (Compound No. 47);
2,3,4-Tri-O-benzyl-6-(adamantanecarboxamido}-N-propyl-D-gluco-δ-lactam (Compound No. 48);
6-N-{[2,4-Difluorophenyl]aminocarbonylamino }-N-propyl-D-gluco-δ-lactam (Compound No. 49);
6-(Adamantane carboxamido)-N-propyl-D-gluco-δ-lactam (Compound No. 50);
2,3,4-Tri-O-benzyl-6-(trifluoromethylacetamido)-N-propyl-D-gluco-δ-lactam (Compound No. 51);
2,3,4-Tri-O-benzyl-6-N-{2-[chloro]-acetyl}-N-propyl-D-gluco-δ-lactam (Compound No. 52);
2,3,4-Tri-O-benzyl-N-propyl-6-{2-[triazolyl]-acetyl}-D-gluco-δ-lactam (Compound No. 53);
2,3,4-Tri-O-benzyl-6-N-{2-[4-(pyrimidyl)piperazinyl]acetyl}-N-propyl-D-gluco-δ-lactam (Compound No. 54);
2,3,4-Tri-O-benzyl-6-N-{2-[4-fluoroanilino]acetyl}-N-propyl-D-gluco-δ-lactam (Compound No. 55);
2,3,4-Tri-O-benzyl-6-{2-[2,6-diketopiperidino]acetyl}-N-propyl-D-gluco-δ-lactam (Compound No. 56);
2,3,4-Tri-O-benzyl-6-N-{2-[4-chlorophenyl-3-(2H,4H)-1,2,4-triazol-3-onyl]acetyl}-N-propyl-D-gluco-δ-lactam (Compound No. 57);
2,3,4-Tri-O-benzyl-6-N-{2-[4-(4-fluorophenyl)-piperazin-1-yl]acetyl}-N-propyl-D-gluco-δ-lactam (Compound No. 58);
2,3,4-Tri-O-benzyl-6-N-{2-[N-(methyl)piperazin-1-yl]acetyl}-N-propyl-D-gluco-δ-lactam (Compound No. 59);
2,3,4-Tri-O-benzyl-6-N-{2-[(4-(4-(chlorophenyl)-piperazin-1-yl)-phenyl)-3(2H,4H)-1,2,4-triazol-3-onyl]acetyl}-H-propyl-D-gluco-δ-lactam (Compound No. 60);
2,3,4-Tri-O-benzyl-6-N-{2-[2,3,4,6-tetra-O-benzyl-1,5-dideoxy-1,5-imino-glucitolyl]acetyl}-N-propyl-D-gluco-δ-lactam (Compound No. 61);
2,3,4-Tri-O-benzyl-6-N-{2-[N-(2,6-diethylphenyl)piperazinyl]acetyl}-N-propyl-D-gluco-δ-lactam (Compound No. 62);
2,3,4-Tri-O-benzyl-6-{2-[1H-isoindole-1,3(2H)-diketo]acetyl}-D-gluco-δ-lactam (Compound No. 63);
2,3,4-Tri-O-benzyl-6-N-{2-[4-(4-chlorophenyl)piperazinyl]acetyl}-N-propyl-D-gluco-δ-lactam (Compound No. 64);
2,3,4-Tri-O-benzyl-6-N-{2-[4-(3-(trifluoromethyl)phenyl)piperazin-1-yl]acetyl}-N-propyl-D-gluco-δ-lactam (Compound No. 65);
N-Propyl-6-{2-[triazolyl]-acetyl}-D-gluco-δ-lactam (Compound No. 66);
6-N-{2-[1,5-Dideoxy-1,5-imino-glucit-6-ol]acetyl}-N-propyl-D-gluco-δ-lactam (Compound No. 67);
6-N-{2-[(N-Methyl)piperazinyl]acetyl}-N-propyl-D-gluco-δ-lactam (Compound No. 68);
N-Allyl-2,3,4-tri-O-benzyl-6-triazolyl-D-gluco-δ-lactam (Compound No. 69);
6-Triazolyl-N-propyl-D-gluco-δ-lactam (Compound No. 70);
N-Allyl-2,3,4-tri-O-benzyl-1,5-dideoxy-1,5-imino-D-glucitol (Compound No. 71);
1,5-Dideoxy-1,5-imino-6-triazolyl-glucitol (Compound No. 72);
1,5-Dideoxy-1,5-imino-N-propyl-6-triazolyl glucitol (Compound No. 73);
2,3,4-Tri-O-benzyl-6-N-[phenylcarbamate]-N-propyl-D-gluco-δ-lactam (Compound No. 74);
2,3,4-Tri-O-benzyl-6-N-{4-[4-chlorophenyl]piperazinyl carboxamido}-N-propyl-D-gluco-δ-lactam (Compound No. 75);
2,3,4-Tri-O-benzyl-6-N-{4-[4-fluorophenyl]piperazinyl carboxamido}-N-propyl-D-gluco-δ-lactam (Compound No. 76);
2,3,4-Tri-O-benzyl-6-N-{1,2-dihydro(2H)-indolyl carboxamido}-N-propyl-D-gluco-δ-lactam (Compound No. 77);
2,3,4-Tri-O-benzyl-6-N-{3-(2-iminocarbonylaminoethyl)indolyl}-N-propyl-D-gluco-δ-lactam (Compound No. 78);
2,3,4-Tri-O-benzyl-6-N-{(1α,5α,6α)-6-acetylamino-azabicyclo[3.1.0]hexyl carboxamido}-N-propyl-D-gluco-δ-lactam (Compound No. 79);
N-{4-[Phenyl]piperazinyl carboxamido}-D-gluco-δ-lactam (Compound No. 80);
2,3,4-Tri-O-benzyl-6-[4-chlorophenyl-3(2H,4H)-1,2,4-triazol-3-onyl]-N-allyl-D-gluco-δ-lactam (Compound No. 81);
N-Allyl-2,3,4-tri-O-benzyl-6-(2,6-diketopiperidino)-D-gluco-δ-lactam (Compound No. 82);
N-Allyl-2,3,4-tri-O-benzyl-6-(1H-isoindole-1,3(2H)-diketo)-D-gluco-δ-lactam (Compound No. 83);
N-Allyl-2,3,4-tri-O-benzyl-6-(2,5-diketopyrrolidino)-D-gluco-δ-lactam (Compound No. 84);
N-Allyl-2,3,4-tri-O-benzyl-1,5-dideoxy-1,5-imino-6-morpholinoglucitol (Compound No. 85);
6-(2,5-Diketopyrrolidino)-N-propyl-D-gluco-δ-actam (Compound No. 86);
6-(2,6-Diketopiperidino)-N-propyl-D-gluco-δ-lactam (Compound No. 87);
N-Propyl-6-[4-chlorophenyl-3(2H,4H)-1,2,4-triazol-3-onyl]-D-gluco-δ-lactam (Compound No. 88);
2,3,4-Tri-O-benzyl-6-(4,6-dichloro-1,3,5-triazin-1-yl)-N-propyl-D-gluco-δ-lactam (Compound No. 89); and
2,3,4-Tri-O-benzyl-6-O-(4,6-dichloro-1,3,5-triazin-1-yl)-N-propyl-D-gluco-δ-lactam (Compound No. 90).

3. A method of inhibiting metastasis of cancer cells in a mammal comprising administering to said mammal a therapeutically effective amount of the compound of claim 1.

4. A method of treating cancer in a mammal comprising administering to said mammal a therapeutically effective amount of a compound of claim 1.

5. A pharmaceutical composition comprising a therapeutically effective amount of a compound as defined in claim 1 and a pharmaceutical acceptable carrier.

6. (canceled)

7. (canceled)

8. A process for preparing a compound of Formula VIII, and its pharmaceutically acceptable salts, enantiomers, diastereomers, N-Oxides, prodrugs, metabolites, polymorphs, or pharmaceutically acceptable solvates, comprising the steps of:
i) reacting a compound of Formula II with p-toluene sulfonyl chloride of Formula III to form a compound of Formula IV, wherein Bn is benzyl,
ii) azidizing the compound of Formula VI to form a compound of Formula V,
iii) reducing the compound of Formula V to form a compound of Formula VI, and
iv) reacting the compound of Formula VII to form a compound Formula VIII, wherein Bn is benzyl, R′ is oxygen or sulfur, and R″ is substituted aryl substituted alkyl, or adamantine:

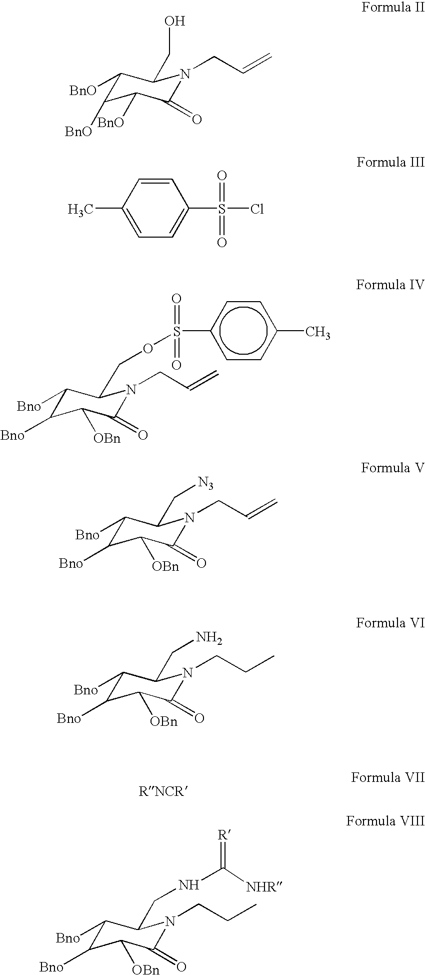

9. (canceled)

10. The process of claim 8 wherein the reaction of the compound of Formula II with the compound of Formula III is carried out in the presence of a base selected from the group consisting of potassium carbonate, cesium carbonate, sodium carbonate, triethylamine and diisopropylamine.

11. (canceled)

12. (canceled)

13. (canceled)

14. (canceled)

15. The process of claim 11, further comprising reducing the compound of Formula VIII to form a compound of Formula IX, wherein Bn is benzyl, R′ is oxygen or sulfur, and R″ is substituted aryl, substituted alkyl, or adamantane.

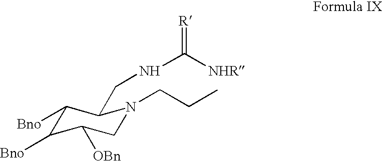

16. The process of claim 11, further comprising debenzylation of debenzylating the compounds of Formula VIII to form a compound of Formula X, wherein R′ is oxygen or sulfur, and R″ is substituted aryl, substituted alkyl, or adamantane.

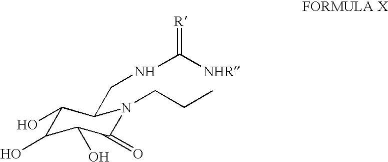

17. The process of claim 16, further comprising silylating the compound of Formula X to form a compound of Formula XI, wherein R′ is oxygen or sulfur, and R″ is substituted aryl, substituted alkyl, or adamantane.

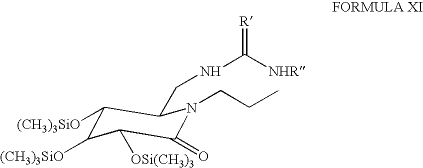

18. The process of claim 17, wherein the silylation of the compound of Formula X is carried out in the presence of a base selected from the group consisting of potassium carbonate, cesium carbonate, sodium carbonate, triethylamine and diisopropylamine.

19. The process of claim 16, further comprising acetylating the compound of Formula X to form a compound of Formula XII, wherein Ac is acetyl, R′ is oxygen or sulfur, and R″ is substituted aryl, substituted alkyl, or adamantane.

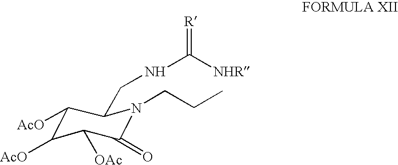

20. A process for preparing a compound of Formula XIII and its pharmaceutically acceptable salts, enantiomers, diastereomers, N-oxides, prodrugs, metabolites, polymorphs or pharmaceutically acceptable solvates thereof which comprises reacting a compound of Formula II with a compound of Formula VII to form a compound of Formula XIII, wherein Bn is benzyl, R′ is oxygen or sulfur, and R″ is substituted aryl, substituted alkyl, or adamantane:

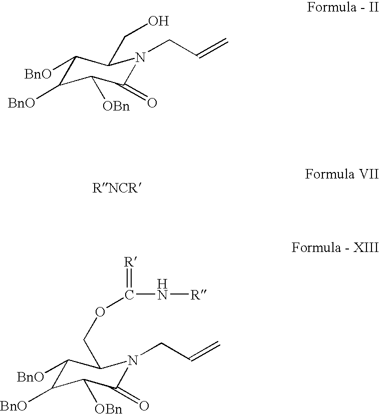

21. (canceled)

22. The process of claim 20, wherein the reaction of the compound of Formula II with the compound of Formula VII is carried out in the presence of a base selected from the group consisting of triethylamine, diusopropylamine, potassium carbonate, cesium carbonate and sodium carbonate.

23. The process of claim 20 further comprising debenzylating the compound of Formula XIII to give a compound of Formula XIV, wherein R′ is oxygen or sulfur, and R″ is substituted aryl, substituted alkyl, or adamantane.

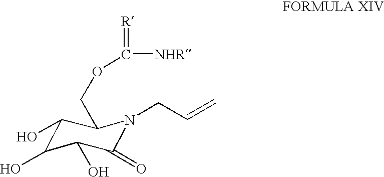

24. The process of claim 20, further comprising reducing the compound of Formula XIII to form a compound of Formula XV, wherein Bn is benzyl, R′ is oxygen or sulfur, and R″ is substituted aryl, substituted alkyl, or adamantane.

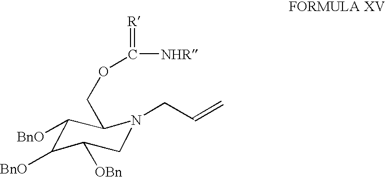

25. A process for preparing a compound of Formula XVII and its pharmaceutically acceptable salts, enantiomers, diastereomers, N-oxides, prodrugs, metabolites, polymorphs or pharmaceutically acceptable solvates thereof, comprising reacting a compound of Formula II with the compound of Formula XVI to form a compound of Formula XVII, wherein Bn is benzyl.

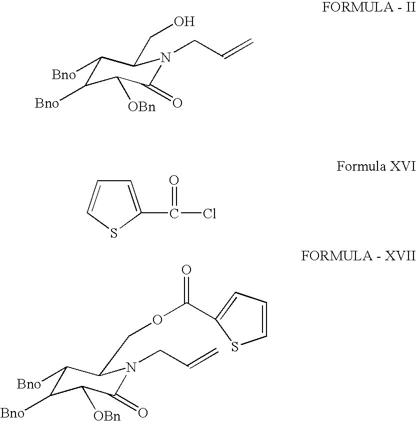

26. The process of claim 25 wherein the reaction of the compound of Formula II with the compound of Formula XVI is carried out in the presence of a base selected from the group consisting of triethylamine and diisopropylamine.

27. A process for preparing a compound of Formula XIX and its pharmaceutically acceptable salts, enantiomers, diastereomers, N-oxides, prodrugs, metabolites, polymorphs or pharmaceutically acceptable solvates thereof comprising condensing a compound of Formula VI with a compound of Formula XVIII to form a compound of Formula XIX, wherein Bn is benzyl, and X′ is selected from the group consisting of thienyl, 2,4-difluorophenyl, and adamantyl.

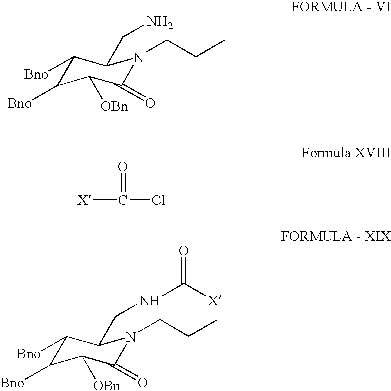

28. (canceled)

29. The process of claim 27 wherein the reaction of the compound of Formula VI and the compound of Formula XVIII is carried out in the presence of a base selected from the group consistin of triethylamine and diisopropylamine.

30. The process of claim 27, further comprising debenzylating the compound of Formula XIX to form a compound of Formula XX, wherein X′ is selected from the group consisting of thienyl, 2,4-difluorophenyl, and adamantyl.

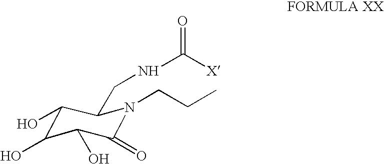

31. A process for preparing a compound of Formula XXII and its pharmaceutically acceptable salts, enantiomers, diastereomers, N-oxides, prodrugs, metabolites, polymorphs or pharmaceutically acceptable solvates thereof comprising reacting a compound of Formula VI with a compound of Formula XXI to form a compound of Formula XXII, wherein Bn is benzyl.

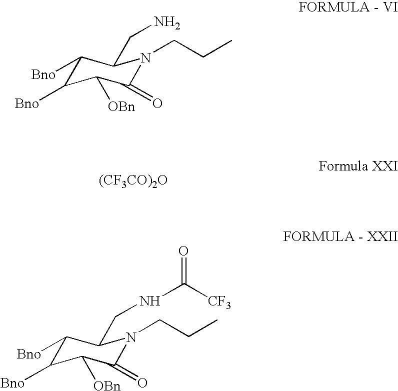

32. (canceled)

33. The process of claim 31 wherein the reaction of the compound of Formula VI with the compound of Formula XXI is carried out in the presence of a base selected from the group consisting of triethylamine and diisopropylamine.

34. A process for preparing a compound of Formula XXIV and its pharmaceutically acceptable salts, enantiomers, diastereomers, N-oxides, prodrugs, metabolites, polymorphs, or pharmaceutically acceptable solvates thereof comprising condensing a compound of Formula VI with a compound of Formula XXIII to form the compound of Formula XXIV, wherein Bn is benzyl.

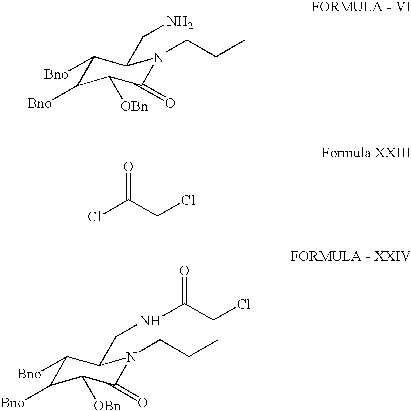

35. (canceled)

36. The process of claim 34 wherein the reaction of the compound of Formula VI with the compound of Formula XXIII is carried out in the presence of a base selected from the group consisting of triethylamine and diisopropylamine.

37. The process of claim 34 further comprising reducing the compound of Formula XXIV with a heterocycle (Het) in the presence of tributylammonium iodide to form a compounds of Formula XXV, wherein Bn is benzyl, and Het is selected from the group consisting of

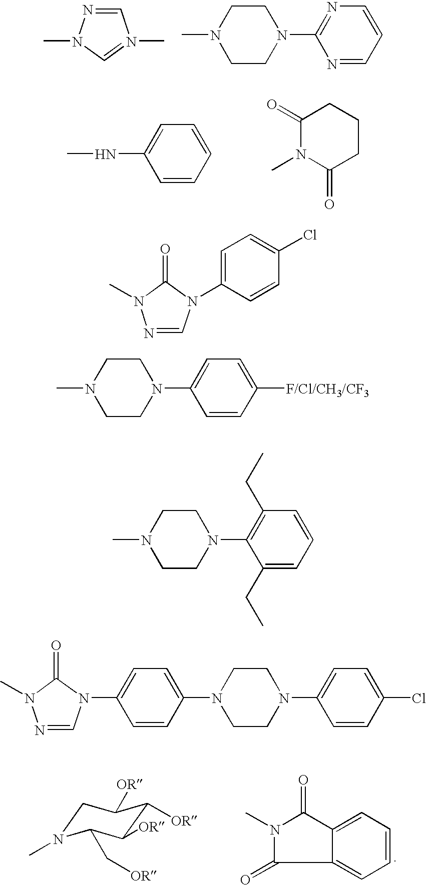

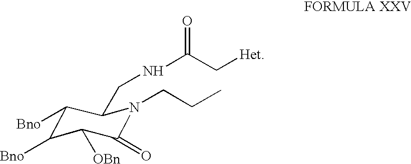

38. (canceled)

39. The process of claim 37, wherein the reaction of the compound of Formula XXIV with the heterocycle (Het) is carried out in the presence of a base selected from the group consisting of triethylamine and diisopropylamine.

40. The process of claim 37, further comprising debenzylating the compound of Formula XXV to form a compound of Formula XXVI, wherein Het is selected from the group consisting of

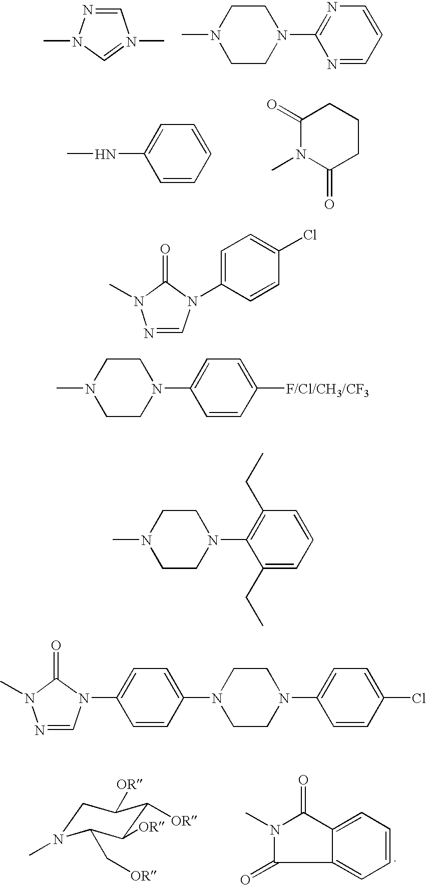

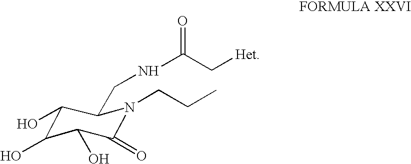

41. A process for preparing a compound of Formula XXVII and its pharmaceutically acceptable salts, enantiomers diastereomers, N-oxides, prodrugs, metabolites, polymorphs or pharmaceutically acceptable solvates thereof comprising reacting a compound of Formula IV with triazole to form a compound of Formula XXVII, wherein Bn is benzyl and Ts is tosylate.

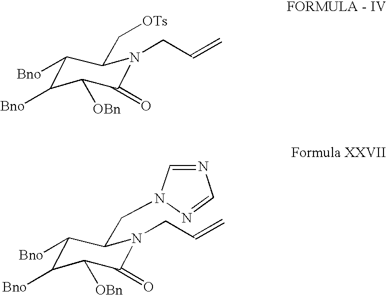

42. (canceled)

43. The process of claim 41 wherein the reaction of compound of Formula IV and triazole is carried out in the presence of a base selected from the group consisting of sodium carbonate, potassium carbonate and cesium carbonate.

44. The process of claim 41 further comprising debenzylating the compound of Formula XXVII to form the compound of Formula XXVIII.

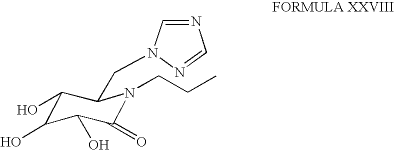

45. The process of claim 41 further comprising reduction of the compound of Formula XXVII to form the compound of Formula XXIX, wherein Bn is benzyl.

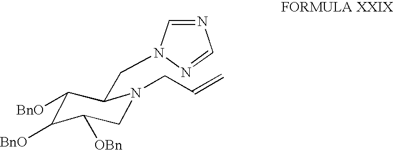

46. The process of claim 45 further comprising the debenzylation of the compound of Formula XXIX to form the compound of Formula XXX.

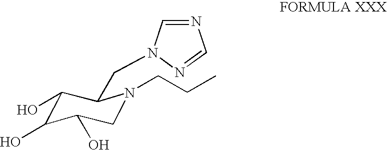

47. The process of claim 45 further comprising catalytic hydrogenation of the compound of Formula XXIX to form a compound of Formula XXXI.

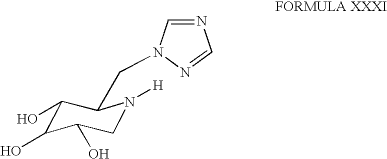

48. A process for preparing a compound of Formula XXXIII and its pharmaceutically acceptable salts, enantiomers, diastereomers, N-oxides, prodrugs, metabolites, polymorphs or pharmaceutically acceptable solvates thereof comprising condensing a compound of Formula VI with a compound of Formula XXXII to form a compound of Formula XXXIII, wherein Bn is benzyl, and X″ is selected from the group consisting of H and NO₂.

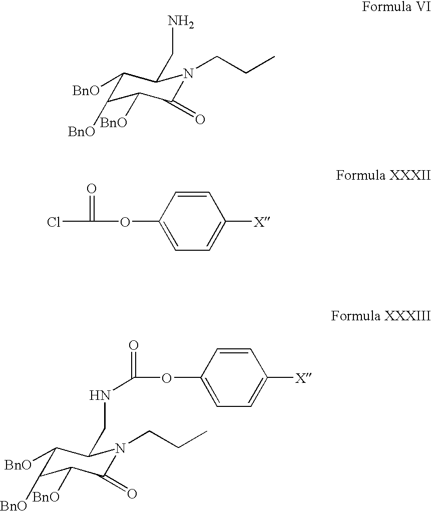

49. (canceled)

50. The process of claim 48 wherein the reaction of the compound of Formula VI with the compound of Formula XXXII is carried out in the presence of a base selected from the group consisting of triethylamine and diisopropylamine.

51. The process of claim 48, further comprising condensing the compound of Formula XXXIII with a compound of Formula XXXIV to form a compound of Formula XXXV, wherein Bn is benzyl, and Y″ is selected from the group consisting of 4-(p-halophenyl)-1-piperazinyl, 4-(phenyl)-1-piperazinyl, 1-indolinyl, 6-acetylamino-3-amino[3.1.0]bicyclohexyl, and 3-alkylaminoindolyl.

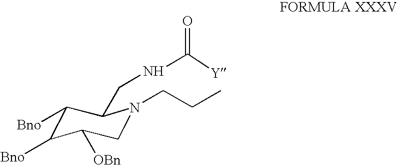

52. The process of claim 51, wherein the reaction of the compound of Formula XXXIII with the compound of Formula XXXIV is carried out in the presence of a base is selected from the group consisting of triethylamine and diisopropylamine.

53. The process of claim 51, further comprising the debenzylating the compound of Formula XXXV to form a compound of Formula XXXVI.

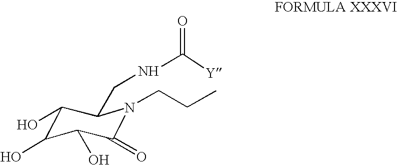

54. A process for preparing a compound of Formula XXXVIII and its pharmaceutically acceptable salts, enantiomers, diastereomers, N-oxides, prodrugs, metabolites, polymorphs, or pharmaceutically acceptable solvates thereof comprising condensing a compound of Formula II with a compound of Formula XXXVII to form a compound of Formula XXXVIII, wherein Bn is benzyl, and X″′ is selected from the group consisting of (p-halophenyl)-triazolonyl, phenyl-triazolonyl, 2,6-dioxo-1-piperidyl, morphlinyl, N-succinamidyl, and 2,5-dioxo-1-pyrrolidinyl.

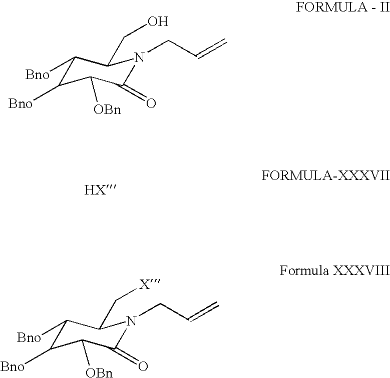

55. (canceled)

56. The process of claim 54 further comprising reducing the compound of Formula XXXVIII to form a compound of Formula XXXIX, wherein Bn is benzyl, and X′″ is selected from the group consisting of (p-halophenyl)-triazolonyl, phenyl-triazolonyl, 2,6-dioxo-1-piperidyl, morphlinyl, N-succinamidyl, and 2,5-dioxo-1-pyrrolidinyl.

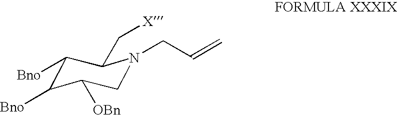

57. The process of claim 54 further comprising debenzylating the compound of Formula XXXVIII to form a compound of Formula XXXX, wherein X′″ is selected from the group consisting of (p-halophenyl)-triazolonyl, phenyl-triazolonyl, 2,6-dioxo-1-piperidyl, morphlinyl, N-succinamidyl, and 2,5-dioxo-1-pyrrolidinyl.

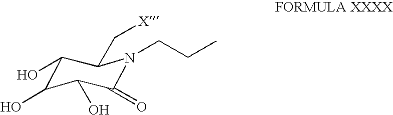

58. A process for preparing a compound of Formula XXXXII and its pharmaceutically acceptable salts, enantiomers, diasteromers, N-oxides, prodrugs, metabolites, polymorphs or pharmaceutically acceptable solvates thereof, comprising condensing a compound Formula VI with a compound of Formula XXXXI to form a compound of Formula XXXXII, wherein Bn is benzyl.

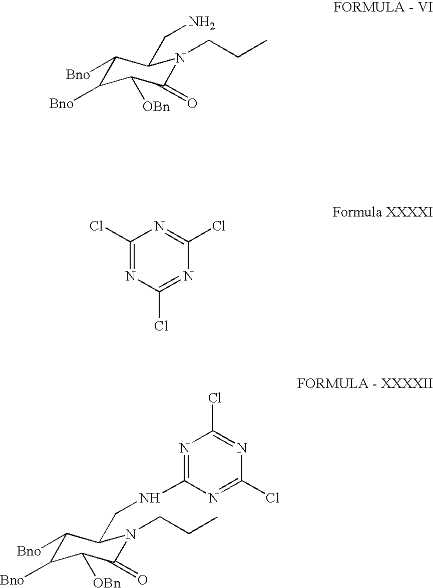

59. The process of claim 58 wherein the reaction of the compound Formula VI with the compound of Formula XXXXI is carried out in the presence of a base selected from the group consisting of potassium carbonate, sodium carbonate and cesium carbonate.

60. (canceled)

61. A process for preparing a compound of Formula XXXXIII and its pharmaceutically acceptable salts, enantiomers, diastereomers, N-oxides, prodrugs, metabolites, polymorphs or pharmaceutically acceptable solvates thereof comprising condensing a compound of Formula II with a compound of Formula XXXXI to form a compound of Formula XXXXIII, wherein Bn is benzyl.

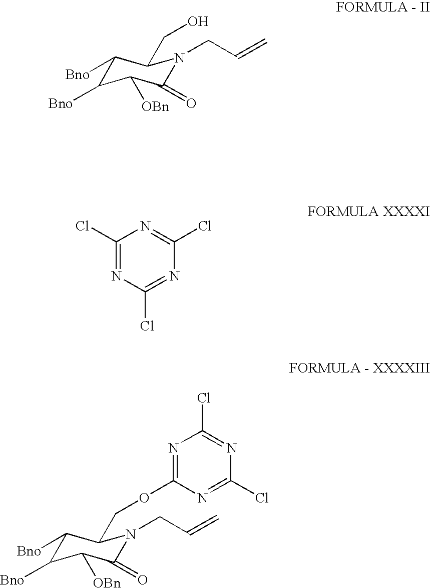

62. (canceled)

63. The process of claim 61 wherein the reaction of the compound of Formula II with the compound of Formula XXXXI is carried out in the presence of a base selected from the group consisting of potassium carbonate sodium carbonate and cesium carbonate.

64. The compound of claim 1, wherein A is propyl, X-G is CH₂, R is benzyl, Y is NH, and Z is CO.

65. The compound of claim 64, wherein P is 4-chlorophenyl, 2-chloroacetyl or 2-(4-fluoroanilino)acetyl.

66. (canceled)

67. (canceled)
